(19) United States
(12) Patent Application Publication
(10) Pub. No.: US 2021/0008048 A1
(43) Pub. Date: Jan. 14, 2021

(54) COMPOSITIONS AND METHODS FOR CANCER TREATMENT

(71) Applicant: Dana-Farber Cancer Institute, Inc., Boston, MA (US)

(72) Inventor: Michael Solomon Goldberg, Brookline, MA (US)

(73) Assignee: Dana-Farber Cancer Institute, Inc., Boston, MA (US)

(21) Appl. No.: 16/982,333

(22) PCT Filed: Mar. 20, 2019
(86) PCT No.: PCT/US2019/023157

§ 371 Date: Sep. 18, 2020

Related U.S. Application Data
(60) Provisional application No. 62/791,481, filed on Jan. 11, 2019.
(60) Provisional application No. 62/645,613, filed on Mar. 20, 2018.

(51) Int. Cl. A61K 31/44; A61K 9/06; A61K 47/36; A61K 47/34; A61K 9/00; A61P 35/00

(52) U.S. Cl. CPC A61K 31/44; A61K 9/06; A61K 47/36; A61P 35/00; A61K 9/0019; A61K 9/0024; A61K 47/34

(57) ABSTRACT

Provided are drug delivery compositions and devices useful for the treatment and/or prevention of cancer and metastatic tumors. For example, a drug delivery composition and/or device is provided that comprises a biodegradable scaffold or biomaterial comprising one or more agents that inhibit one or more proinflammatory pathways, such as one or more immune responses mediated by a p38 mitogen-activated protein kinase (MAPK) pathway. In some embodiments, a drug delivery composition and/or device may further comprise one or more agents that activate the innate immune system (e.g., STING agonists) and/or the adaptive immune system (e.g., anti-PD-1 antibodies). In some embodiments, a drug delivery composition and/or device may include a cytokine (e.g., IL-15 superagonist). In some embodiments, a drug delivery composition and/or device can be administered to a tumor resection site (e.g., a void volume resulting from a tumor resection). Such intraoperative administration can prevent tumor regrowth and/or tumor metastasis. Also provided are methods of making drug delivery compositions and devices as well as kits containing materials to provide the same.

### CROSS REFERENCE TO RELATED APPLICATIONS

This application claims priority under 35 U.S.C. § 119(e) to U.S. Provisional Patent Application No. 62/645,613, filed Mar. 20, 2018, and U.S. Provisional Patent Application No. 62/791,481, filed Jan. 11, 2019, each of which is incorporated herein by reference in its entirety.

### BACKGROUND

Systemic administration of medication, nutrition, or other substances into the circulatory system affects the entire body. Systemic routes of administration include enteral (e.g., oral dosage resulting in absorption of the drug through the gastrointestinal tract) and parenteral (e.g., intravenous, intramuscular, and subcutaneous injections) administration. Administration of immunotherapeutics typically relies on these systemic administration routes, which can lead to unwanted side effects. In some instances, certain promising therapeutics are extremely difficult to develop due to associated toxicities and the limitations of current administration methods and systems.

Surgery is often the first-line of treatment for solid tumor cancers and is generally used in combination with systemic administration of anti-cancer therapy. However, surgery-induced immunosuppression has been implicated in the development of post-operative septic complications and tumor metastasis due to changes in a variety of metabolic and endocrine responses, ultimately resulting in the death of many patients (Smyth, M. J. et al., Nature Reviews Clinical Oncology, 2016, 13, 143-158).

### SUMMARY

Systemic administration of immunotherapies can result in adverse side effects, e.g., inducing toxicities that are undesirable for non-cancerous cells and/or tissues such as non-tumor-specific immune cells, and/or requiring high doses in order to achieve sufficient concentration at a target site to induce a therapeutic response; and surgical resection of tumors can result in immunosuppression. Surgery can also induce cellular stress, which may involve, for example, activation of one or more physiological responses that promote wound healing after injury. Such responses include, e.g., activation of neural, inflammatory, and/or pro-angiogenic signalling pathways, which can also promote the growth and/or metastatic spread of cancer. Inflammatory changes that may occur at a surgical site following tumor resection can include, e.g., recruitment of immune and/or inflammatory cell type(s) and/or release of humoral factor(s). Local inflammatory wound response and systemic inflammation processes together might activate dormant micrometastases or induce the propagation of residual cancer cells, thus increasing the risk of cancer recurrence.

The present disclosure, among other things, provides insights that include identification of the source of a problem with certain prior technologies including, for example, certain conventional approaches to cancer treatment. For example, the present disclosure appreciates that certain adverse events that can be associated with systemic administration of immunotherapeutic agents (e.g., skin rashes, hepatitis, diarrhea, colitis, hypophysitis, thyroiditis, and adrenal insufficiency) may be immune-related and may, at least in part, be attributable to exposure of non-tumor-specific immune cells to the systemically-administered immunotherapeutic drug. Among other things, the present disclosure appreciates that the high doses typically required for systemic administration to achieve sufficient concentration in the tumor to induce a desired response may contribute to and/or be responsible for, such undesirable effects. The present technology provides systems that solve such problems, among other things by providing localized delivery of immunotherapeutic agents which, among other things, can improve efficacy by concentrating the action of the drug where it is needed.

Moreover, the present disclosure provides insights that certain immunomodulatory agents traditionally used to treat autoimmune-type pathologies could be useful in the treatment of cancer if administered as described herein, notwithstanding that off-target toxicity would have otherwise been expected to be in opposition to those anticipated for an anti-cancer immunomodulatory compound. Thus, the present disclosure teaches usefulness for cancer therapy of agents previously not considered useful and furthermore teaches delivery and dosing strategies that are particularly effective and/or desirable for these and other agents.

The present disclosure recognizes, among other things, that inhibiting one or more proinflammatory immune responses mediated by a p38 mitogen-activated protein kinase (MAPK) pathway (e.g., by administration of a p38 MAPK inhibitor) at a tumor resection site can reduce the risk of cancer recurrence and thus prolong survival. The present disclosure provides drug delivery systems that can localize delivery of one or more immunomodulatory agents to a target site (e.g., a site at which a tumor has been removed and/or cancer cells have been treated or killed, e.g., by chemotherapy or radiation) and thereby concentrate the action of the immunomodulatory agents to a target site in need thereof. Such drug delivery systems can be particularly useful for treating cancer. In particular, the drug delivery systems deliver one or more therapeutic agents that act on (e.g., inhibit) one or more proinflammatory pathways (e.g., proinflammatory immune response mediated by a p38 MAPK pathway; see, for example, FIGS. 4-6), e.g., following a tumor resection, for the treatment of cancer, such as, for example, by preventing (e.g., delaying onset of, reducing extent of) tumor recurrence and/or metastasis, in some embodiments while minimizing adverse side effects and/or systemic exposure.

In some aspects, provided are methods comprising intraoperatively administering at a target site (e.g., a tumor resection site) of a subject suffering from cancer, a composition comprising a biomaterial and an inhibitor of a proinflammatory immune response mediated by a p38 mitogen-activated protein kinase (MAPK) pathway.

In certain embodiments, the biomaterial has a storage modulus of about 500 Pa to about 50,000 Pa. In certain embodiments, the biomaterial is or comprises a hydrogel. In certain embodiments, the biomaterial is or comprises hyaluronic acid. In certain embodiments, the biomaterial is or comprises a crosslinked hyaluronic acid. In certain embodiments, the biomaterial is or comprises a hyaluronic acid crosslinked with a polyethylene glycol crosslinker.

In certain embodiments, the method does not include administering adoptive transfer of T cells to the subject. In certain embodiments, the method does not include administering a tumor antigen to the subject. In certain embodiments, the method does not include administering a microparticle to the subject.

In certain embodiments, the inhibitor is a p38 α/β MAPK inhibitor that binds to an ATP and/or allosteric binding site of p38 MAPK. In certain embodiments, the p38 α/β MAPK inhibitor is losmapimod.

In certain embodiments, the composition further comprises an activator of innate immunity. In certain embodiments, the activator of innate immunity is a stimulator of interferon genes (STING) agonist. In certain embodiments, the activator of innate immunity is a Toll-like receptor (TLR) 7 and/or TLR8 (“TLR7/8”) agonist. In certain embodiments, the composition further comprises an activator of adaptive immunity and/or a cytokine that modulates T cells, natural killer (NK) cells, monocytes, and/or dendritic cells. In certain embodiments, the composition further comprises a cytokine that modulates T cells, NK cells, monocytes, and/or dendritic cells. Examples of such a cytokine include, but are not limited to an IL-15 superagonist, IFN-α, IFN-β, IFN-γ, and combinations thereof. In certain embodiments, the composition further comprises an inhibitor of cyclooxygenase (COX), including, e.g., a COX-2 inhibitor.

Those skilled in the art will appreciate that certain COX inhibitors and/or other anti-inflammatory agents (e.g., non-sterodial anti-inflammatory drugs (NSAIDs) and/or anti-inflammatory analgesics) may act as modulators (e.g., inhibitors) of a p38 MAPK pathway or component(s) thereof (see, e.g., as described in Esposito et al., “Non-steroidal anti-inflammatory drugs in Parkinson's disease” Experimental Neurology 205: 295-312 (2007); Desai et al., “Mechanisms of Phytonutrient Modulation of Cyclooxygenase-2 (COX-2) and Inflammation Related to Cancer” Nutrition and Cancer, 70: 350-375 (2018); Huang et al., “MAPK/ERK signal pathway involved expression of COX-2 and VEGF by IL-1beta induced in human endometriosis stomal cells in vitro” Int J Clin Exp Pathol, 6: 2129-2136 (2013); and Di Mari et al., “HETEs enhance IL-1-mediated COX-2 expression via augmentation of message stability in human colonic myofibroblasts” Am J Physiol-Gastrointest Liver Physiol., 293: 2092-2101 (2007)). Thus, in some embodiments, a COX-2 inhibitor or other anti-inflammatory agent (e.g., non-sterodial anti-inflammatory drugs (NSAIDs) and/or anti-inflammatory analgesics) may be (and/or may be used as) a p38 MAPK inhibitor as described herein; alternatively or additionally, in some embodiments, such a COX-2 inhibitor or other anti-inflammatory agent (e.g., anti-inflammatory analgesics) may be utilized in combination with another p38 MAPK inhibitor as described herein.

In certain embodiments, the biomaterial forms a matrix or depot and the inhibitor is within the biomaterial. In certain embodiments, the inhibitor is released by diffusion through the biomaterial. In certain embodiments, the biomaterial is biodegradable in vivo. In certain embodiments, the biomaterial is characterized in that, when tested in vivo by implanting a biomaterial at a mammary fat pad of a mouse subject, less than or equal to 10% of the biomaterial remains in vivo 4 months after the implantation. In certain embodiments, the biomaterial is characterized in that, when tested in vitro by placing a composition comprising a biomaterial and losmapimod in PBS (pH 7.4), less than 100% of the losmapimod is released within 3 hours from the biomaterial. In certain embodiments, the biomaterial is characterized in that, when tested in vivo by implanting a composition comprising a biomaterial and losmapimod at a mammary fat pad of a mouse subject, less than or equal to 50% of the losmapimod is released in vivo 8 hours after the implantation. In certain embodiments, the biomaterial is characterized in that it extends release of the inhibitor so that, when assessed at 24 hours after administration, more inhibitor is present in the tumor resection site than is observed when the inhibitor is administered in solution.

In certain embodiments, administering is by implantation. In certain embodiments, administering is by injection. In certain embodiments, administering comprises injecting one or more precursor components of the biomaterial and permitting the biomaterial to form at the tumor resection site. In certain embodiments where the target site is a tumor resection site, the tumor resection site is characterized by the absence of gross residual tumor antigen. In certain embodiments, the cancer is metastatic cancer. In certain embodiments, the method further comprises monitoring at least one metastatic site in the subject after administering the composition.

The details of certain embodiments of the invention are set forth herein. Other features, objects, and advantages of the invention will be apparent from the Detailed Description, Examples, and Claims.

### Definitions

As used herein, the term “salt” refers to any and all salts and encompasses pharmaceutically acceptable salts.

The term “pharmaceutically acceptable salt” refers to those salts which are, within the scope of sound medical judgment, suitable for use in contact with the tissues of, for example, humans and/or animals without undue toxicity, irritation, allergic response, and the like and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts that may be utilized in accordance with certain embodiments of the present disclosure may include, for example, those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁-C₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “polymer” is given its ordinary meaning as used in the art, i.e., a molecular structure comprising one or more repeat units (monomers), connected by covalent bonds. The repeat units may all be identical, or, in some cases, there may be more than one type of repeat unit present within the polymer. In certain embodiments, a polymer is naturally occurring. In certain embodiments, a polymer is synthetic (i.e., not naturally occurring). In some embodiments, a polymer for use in accordance with the present disclosure is a polypeptide. In some embodiments, a polymer for use in accordance with the present disclosure is not a nucleic acid.

The term “bioadhesive” refers to a biocompatible agent that can adhere to a target surface, e.g., a tissue surface. In some embodiments, a bioadhesive can adhere to a target surface, e.g., a tissue surface, and retain on the target surface, e.g., for a period of time. In some embodiments, a bioadhesive may be biodegradable. In some embodiments, a bioadhesive may be a natural agent, which may have been prepared or obtained, for example, by isolation or by synthesis; in some embodiments, a bioadhesive may be a non-natural agent, e.g., as may have been designed and/or manufactured by the hand of man (e.g., by processing, synthetic, and/or recombinant production, depending on the agent, as will be understood by those skilled in the art. In some particular embodiments, a bioadhesive may be or comprise a polymeric material, e.g., as may be comprised of or contain a plurality of monomers such as sugars. Certain exemplary bioadhesives include a variety of FDA-approved agents such as, for example, cyanoacrylates (Dermabond, 2-Octyl cyanoacrylate; Indermil, n-Butyl-2-cyanoacrylate; Histoacryl and Histoacryl Blue, n-Butyl-2-cyanoacrylate), albumin and glutaraldehyde (BioGlue™, bovine serume albumin and 10% glutaraldehyde), fibrin glue (Tisseel™, human pooled plasma fibrinogen and thrombin; Evicel™, human pooled plasma fibrinogen and thrombin; Vitagel™, autologous plasma fibrinogen and thrombin; Cryoseal™ system, autologous plasma fibrinogen and thrombin), gelatin and/or resorcinol crosslinked by formaldehyde and/or glutaraldehyde, polysaccharaide-based adhesives (gelatin, collagen, dextran, chitosan, alginate), PEG, acrylates, polyamines, or urethane derivatives (isocyanate-terminated prepolymer, and/or combinations thereof. Other examples of bioadhesives that are known in the art, e.g., as described in Mehdizadeh and Yang “Design Strategies and Applications of Tissue Bioadhesives” Macromol Biosci 13:271-288 (2013), can be used for the purposes of the methods described herein. In some embodiments, a bioadhesive can be a degradable bioadhesive. Examples of such a degradable bioadhesive include, but are not limited to, fibrin glues, gelatin-resorcinol-formaldehyde/glutaraldehyde glues, poly(ethylene glycol) (PEG)-based hydrogel adhesives, polysaccharide adhesives, polypeptide adhesives, polymeric adhesives, biomimetic bioadhesives, and ones described in Bhagat and Becker “Degradable Adhesives for Surgery and Tissue Engineering” Biomacromolecules 18: 3009-3039 (2017).

The term “cross-linker” refers to an agent that links one entity (e.g., one polymer chain) to another entity (e.g., another polymer chain). In some embodiments, linkage (i.e., the “cross-link”) between two entities is or comprises a covalent bond. In some embodiments, linkage between two entities is or comprises a non-covalent association. For example, in some embodiments, linkage between two entities is or comprises an ionic bond or interaction. In some embodiments, a cross-linker is a small molecule (e.g., dialdehydes or genipin) for inducing formation of a covalent bond between an aldehyde and an amino group. In some embodiments, a cross-linker comprises a photo-sensitive functional group. In some embodiments, a cross-linker comprise a pH-sensitive functional group. In some embodiments, a cross-linker comprise a thermal-sensitive functional group.

The term “solvate”, as used herein, has its art-understood meaning and refers to an aggregate of a compound (which may, for example, be a salt form of the compound) and one or more solvent atoms or molecules. In some embodiments, a solvate is a liquid. In some embodiments, a solvate is a solid form (e.g., a crystalline form). In some embodiments, a solid-form solvate is amenable to isolation. In some embodiments, association between solvent atom(s) and compound in a solvate is a non-covalent association. In some embodiments, such association is or comprises hydrogen bonding, van der Waals interactions, or combinations thereof. In some embodiments, a solvent whose atom(s) is/are included in a solvate may be or comprise one or more of water, methanol, ethanol, acetic acid, DMSO, THF, diethyl ether, and the like. Suitable solvates may be pharmaceutically acceptable solvates; in some particular embodiments, solvates are hydrates, ethanolates, or methanolates. In some embodiments, a solvate may be a stoichiometric solvate or a non-stoichiometric solvate.

The term “hydrate”, as used herein, has its art-understood meaning and refers to an aggregate of a compound (which may, for example be a salt form of the compound) and one or more water molecules. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2H₂O) and hexahydrates (R.6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting point, density, hardness, crystal shape, optical properties, electrical properties, stability, solubility, bioavailability, etc. Recrystallization solvent, rate of crystallization, storage temperature, and other factors may cause one crystal form to dominate a given preparation. Various polymorphs of a compound can typically be prepared by crystallization under different conditions.

The term “co-crystal” refers to a crystalline structure composed of at least two components. In certain embodiments, a co-crystal contains a compound of interest (e.g., ones disclosed herein) and one or more other component(s), such as, for example, one or more atoms, ions, or molecules (e.g., solvent molecules). In certain embodiments, a co-crystal contains a compound of interest and one or more solvent molecules. In certain embodiments, a co-crystal contains a compound of interest and one or more acid or base.

The term “prodrug” refers to a form of an active compound that includes one or more cleavable group(s) that is/are removed by solvolysis or under physiological conditions, so that the active compound is released. Exemplary prodrug forms include, but are not limited to, choline ester derivatives and the like as well as N-alkylmorpholine esters and the like. In some embodiments, a prodrug may be an acid derivative, such as is known in the art, such as, for example, esters prepared by reaction of the parent acid with a suitable alcohol, amides prepared by reaction of the parent acid compound with a substituted or unsubstituted amine, acid anhydrides, or mixed anhydrides. Simple aliphatic or aromatic esters, amides, and anhydrides derived from acidic groups pendant on a compound of interest are particular examples of prodrug forms. In some cases, it may be desirable to prepare double ester-type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of a compound of interest.

A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or a non-human animal, for example, a mammal (e.g., a primate (e.g., cynomolgus monkey, rhesus monkey); a domestic animal such as a cow, pig, horse, sheep, goat, cat, and/or dog; and/or a bird (e.g., a chicken, duck, goose, and/or turkey). In certain embodiments, the animal is a mammal (e.g., at any stage of development). In some embodiments, an animal (e.g., a non-human animal) may be a transgenic or genetically engineered animal. In some embodiments, a subject is a tumor resection subject, e.g., a subject who has recently undergone tumor resection. In some embodiments, a tumor resection subject is a subject who has undergone tumor resection in less than 72 hours (including, e.g., less than 48 hours, less than 24 hours, less than 12 hours, less than 6 hours, or lower), prior to receiving a drug delivery composition or device described herein. In some embodiments, a tumor resection subject is a subject who has undergone tumor resection in less than 48 hours, prior to receiving a drug delivery composition or device described herein. In some embodiments, a tumor resection subject is a subject who has undergone tumor resection in less than 24 hours, prior to receiving a drug delivery composition or device described herein. In some embodiments, a tumor resection subject is a subject who has undergone tumor resection in less than 12 hours, prior to receiving a drug delivery composition or device described herein.

The term “biological sample” refers to any sample, including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration” refer to implanting, absorbing, ingesting, injecting, inhaling, or otherwise introducing a drug delivery composition as described herein.

The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein. In some embodiments, treatment may be administered after one or more signs or symptoms have developed or have been observed. Treatment may also be continued after symptoms have resolved, for example, to delay or prevent recurrence and/or spread.

The terms “condition,” “disease,” and “disorder” are used interchangeably.

An “effective amount” is an amount sufficient to elicit a desired biological response, e.g., treating a condition from which a subject may be suffering. As will be appreciated by those of ordinary skill in this art, the effective amount of drug delivery composition may vary depending on such factors as the desired biological endpoint, the pharmacokinetics of the therapeutic agents in the composition, the condition being treated, and the age and health of the subject. An effective amount encompasses therapeutic and prophylactic treatment. For example, in treating cancer, an effective amount may prevent tumor regrowth, reduce the tumor burden, or stop the growth or spread of a tumor. Those skilled in the art will appreciate that an effective amount need not be contained in a single dosage form. Rather, administration of an effective amount may involve administration of a plurality of doses, potentially over time (e.g., according to a dosing regimen).

A “therapeutically effective amount” is an amount sufficient to provide a therapeutic benefit in the treatment of a condition, which therapeutic benefit may be or comprise, for example, reduction in frequency and/or severity, and/or delay of onset of one or more features or symptoms associated with the condition. A therapeutically effective amount means an amount of therapeutic agent(s), alone or in combination with other therapies, that provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent. Those skilled in the art will appreciate that a therapeutically effective amount need not be contained in a single dosage form. Rather, administration of an effective amount may involve administration of a plurality of doses, potentially over time (e.g., according to a dosing regimen).

A “prophylactically effective amount” is an amount sufficient to prevent (e.g., significantly delay onset or recurrence of one or more symptoms or characteristics of, for example so that it/they is/are not detected at a time point at which they would be expected absent administration of the amount) a condition. A prophylactically effective amount of a composition means an amount of therapeutic agent(s), alone or in combination with other agents, that provides a prophylactic benefit in the prevention of the condition. The term “prophylactically effective amount” can encompass an amount that improves overall prophylaxis or enhances the prophylactic efficacy of another prophylactic agent. Those skilled in the art will appreciate that a prohylactially effective amount need not be contained in a single dosage form. Rather, administration of an effective amount may involve administration of a plurality of doses, potentially over time (e.g., according to a dosing regimen).

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis or diseases associated with angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Of particular interest in the context of some embodiments of the present disclosure are cancers treated by cell killing and/or removal therapies (e.g., surgical resection and/or certain chemotherapeutic therapies such as cytotoxic therapies, etc). In some embodiments, a cancer that is treated in accordance with the present disclosure is one that has been surgically resected (i.e., for which at least one tumor has been surgically resected). In some embodiments, a cancer that is treated in accordance with the present disclosure is one for which resection is standard of care. In some embodiments, a cancer that is treated in accordance with the present disclosure is one that has metastasized. In certain embodiments, exemplary cancers may include one or more of acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); parathryroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget's disease of the vulva).

The term “immunotherapy” refers to a therapeutic agent that promotes the treatment of a disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immuntherapies are typically, but not always, biotherapeutic agents. Numerous immunotherapies are used to treat cancer. These include, but are not limited to, monoclonal antibodies, adoptive cell transfer, cytokines, chemokines, vaccines, small molecule inhibitors, and small molecule agonists. For example, useful immunotherapies may include, but are not limited to, inducers of type I interferon, interferons, stimulator of interferon genes (STING) agonists, TLR7/8 agonists, IL-15 superagonists, anti-PD-1 antibodies, anti-CD137 antibodies, and anti-CTLA-4 antibodies.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

The term “antibody” refers to a functional component of serum and is often referred to either as a collection of molecules (antibodies or immunoglobulins) or as one molecule (the antibody molecule or immunoglobulin molecule). An antibody is capable of binding to or reacting with a specific antigenic determinant (the antigen or the antigenic epitope), which in turn may lead to induction of immunological effector mechanisms. An individual antibody is usually regarded as monospecific, and a composition of antibodies may be monoclonal (i.e., consisting of identical antibody molecules) or polyclonal (i.e., consisting of two or more different antibodies reacting with the same or different epitopes on the same antigen or even on distinct, different antigens). Each antibody has a unique structure that enables it to bind specifically to its corresponding antigen, and all natural antibodies have the same overall basic structure of two identical light chains and two identical heavy chains. Antibodies are also known collectively as immunoglobulins. An antibody may be of human or non-human (for example, rodent such as murine, dog, camel, etc) origin (e.g., may have a sequence originally developed in a human or non-human cell or organism), or may be or comprise a chimeric, humanized, reshaped, or reformatted antibody based, e.g., on a such a human or non-human antibody (or, in some embodiments, on an antigen-binding portion thereof).

In some embodiments, as will be clear from context, the term “antibody” as used herein encompasses formats that include epitope-binding sequences of an antibody, which such formats include, for example chimeric and/or single chain antibodies (e.g., a nanobody or Fcab), as well as binding fragments of antibodies, such as Fab, Fv fragments or single chain Fv (scFv) fragments, as well as multimeric forms such as dimeric IgA molecules or pentavalent IgM molecules. Also included are bispecific antibodies, bispecific T cell engagers (BiTEs), immune mobilixing monoclonal T cell receptors against cancer (ImmTACs), dual-affinity re-targeting (DART); alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies.

The term “small molecule” or “small molecule therapeutic” refers to a molecule, whether naturally occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, a small molecule is a therapeutically active agent such as a drug (e.g., a molecule approved by the U.S. Food and Drug Administration as provided in the Code of Federal Regulations (C.F.R.)). A small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, a small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to an agent having one or more therapeutic properties that produce a desired, usually beneficial, effect. For example, a therapeutic agent may treat, ameliorate, and/or prevent disease. In some embodiments, a therapeutic agent may be or comprise a biologic, a small molecule, or a combination thereof.

The term “chemotherapeutic agent” refers to a therapeutic agent known to be of use in chemotherapy for cancer.

The term “targeted agent” refers to an anticancer agent that blocks the growth and spread of cancer by interfering with specific molecules (“molecular targets”) that are involved in the growth, progression, and spread of cancer. Targeted agents are sometimes called “targeted cancer therapies,” “molecularly targeted drugs,” “molecularly targeted therapies,” or “precision medicines.” Targeted agents differ from standard chemotherapy in that targeted agents act on specific molecular targets that are associated with cancer, whereas many chemotherapeutic agents act on all rapidly dividing cells (e.g., whether or not the cells are cancerous). Targeted agents are deliberately chosen or designed to interact with their target, whereas many standard chemotherapies are identified because they kill cells.

The term “biomaterial” refers to a biocompatible substance characterized in that it can be administered to a subject for a medical purpose (e.g., therapeutic, diagnostic) without eliciting an unacceptable (according to sound medical judgement) reaction. Biomaterials can be obtained or derived from nature or synthesized. In some embodiments, a biomaterial can be in a form of gel. In some embodiments, a biomaterial can be in an injectable format. For example, a biomaterial can comprise precursor components of a gel to be formed in situ (e.g., upon administration to a subject).

The term “hydrogel” refers to a material formed from a network of polymer chains that are hydrophilic, sometimes found as a colloidal gel in which an aqueous phase is the dispersion medium. In some embodiments, hydrogels are highly absorbent (e.g., they can absorb and/or retain over 90% water) natural or synthetic polymeric networks. In some embodiments, hydrogels possess a degree of flexibility similar to natural tissue, for example due to their significant water content.

The terms “implantable,” “implantation,” “implanting,” and “implant” refer to positioning a drug delivery composition at a specific location in a subject, such as within a tumor resection site or in a sentinel lymph node, and typically by general surgical methods.

The term “biocompatible” refers to a material that is substantially non-toxic in the in vivo environment of its intended use and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity, and/or immunogenicity. A biocompatible structure or material, when introduced into a majority of patients, will not cause an undesirably adverse, long-lived, or escalating biological reaction or response and is distinguished from a mild, transient inflammation, which typically accompanies surgery or implantation of foreign objects into a living organism.

The term “antagonist” refers to an agent that (i) decreases or suppresses one or more effects of another agent; and/or (ii) decreases or suppresses one or more biological events. In some embodiments, an antagonist may reduce level and/or activity or one or more agents that it targets. In various embodiments, antagonists may be or include agents of various chemical class including, for example, small molecules, polypeptides, nucleic acids, carbohydrates, lipids, metals, and/or other entity that shows the relevant antagonistic activity. An antagonist may be direct (in which case it exerts its influence directly upon its target) or indirect (in which case it exerts its influence by other than binding to its target; e.g., by interacting with a regulator of the target, for example so that level or activity of the target is altered). In some embodiments, an antagonist may be a receptor antagonist, e.g., a receptor ligand or drug that blocks or dampens a biological response by binding to and blocking a receptor rather than activating it like an agonist.

The term “agonist” refers to an agent that (i) increases or induces one or more effects of another agent; and/or (ii) increases or induces one or more biological events. In some embodiments, an agonist may increase level and/or activity or one or more agents that it targets. In various embodiments, agonists may be or include agents of various chemical class including, for example, small molecules, polypeptides, nucleic acids, carbohydrates, lipids, metals, and/or other entity that shows the relevant agonistic activity. An agonist may be direct (in which case it exerts its influence directly upon its target) or indirect (in which case it exerts its influence by other than binding to its target; e.g., by interacting with a regulator of the target, for example so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

The term “inhibit” or “inhibition” in the context of modulating level (e.g., expression and/or activity) of a target (e.g., p38 MAPK) is not limited to only total inhibition. Thus, in some embodiments, partial inhibition or relative reduction is included within the scope of the term “inhibition.” In some embodiments, the term refers to a reduction of the level (e.g., expression, and/or activity) of a target (e.g., p38 MAPK) to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target.

As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

The term “inhibitor of a proinflammatory pathway” as used herein, in some embodiments, refers to an agent that prevents recruitment of immunosuppressive cells or prevents acute inflammation. Such acute inflammation and/or recruitment of immunosuppressive cells can occur after local trauma, including that which is caused by surgery. In some embodiments, an inhibitor of a proinflammatory pathway may inhibit, for example, an immune response that induces inflammation, including, e.g., production of proinflammatory cytokines (e.g., TNF-alpha, IL-1β, and IL-6), increased activity and/or proliferation of Th1 cells, recruitment of myeloid cells, etc.

The term “proinflammatory immune response” as used herein refers to an immune response that induces inflammation, including, e.g., production of proinflammatory cytokines (e.g., TNF-alpha, IL-1β, and IL-6), increased activity and/or proliferation of Th1 cells, recruitment of myeloid cells, etc. In some embodiments, a proinflammatory immune response may be or comprise one or both of acute inflammation and chronic inflammation.

The term “activator of innate immune response” refers to an agent that activates the innate immune system. Such activation can stimulate the expression of molecules that initiate an inflammatory response and/or help to induce adaptive immune responses, leading to the development of antigen-specific acquired immunity. Activation of the innate immune system can lead to cytokine production, proliferation, and survival as well as improved T cell priming by enhancing presentation of antigens and expression of co-stimulatory molecules by antigen-presenting cells.

The term “activator of adaptive immune response” refers to an agent that activates the adaptive immune system. Such activation can restore antitumor function by neutralizing inhibitory immune checkpoints or by triggering co-stimulatory receptors, ultimately generating helper and/or effector T cell responses against immunogenic antigens expressed by cancer cells and producing memory B cell and/or T cell populations. In certain embodiments, the activator of adaptive immune response involves modulation of adaptive immune response and/or leukocyte trafficking.

The term “modulator of macrophage effector function” refers to an agent that activates macrophage effector function or depletes immunosuppressive macrophages or macrophage-derived suppressor cells. Such potentiation can mobilize macrophage and myeloid components to destroy the tumor and its stroma, including the tumor vasculature. Macrophages can be induced to secrete antitumor cytokines and/or to perform phagocytosis, including antibody-dependent cellular phagocytosis.

As used herein, the terms “sustained release” and “extended release” are equivalent terms. The compositions and devices of the present disclosure may release therapeutic agents over a period of time. The terms “sustained” and “extended” may mean that one or more therapeutic agents is/are released from a biomaterial on a timescale ranging from 5 minutes to several months. In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of one or more therapeutic agents is/are released from a biomaterial over a period of 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, 10 minutes, or 5 minutes. In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of one or more therapeutic agents is/are released from a biomaterial over a period of 1 day, 18 hours, 12 hours, 8 hours, 6 hours, 4 hours, 3 hours, 2 hours, 1 hours, 45 minutes, 30 minutes, 20 minutes, 15 minutes, 10 minutes, or 5 minutes. In some embodiments, the extent of sustained release or extended release can be characterized in vitro or in vivo. For example, in some embodiments, the release kinetics can be tested in vitro by placing a composition comprising a biomaterial and a therapeutic agent (e.g., a p38 MAPK inhibitor) in an aqueous buffered solution (e.g., PBS at pH 7.4). In some embodiments, when a composition comprising a biomaterial and a therapeutic agent (e.g., a p38 MAPK inhibitor) is placed in an aqueous buffered solution (e.g., PBS at pH 7.4), less than 100% or lower (including, e.g., less than or equal to 90%, less than or equal to 80%, less than or equal to 70%, less than or equal to 50% or lower) of the therapeutic agent is released within 3 hours from the biomaterial. In some embodiments, the release kinetics can be tested in vivo by implanting a composition comprising a biomaterial and a therapeutic agent (e.g., a p38 MAPK inhibitor) at a target site (e.g., mammary fat pad) of an animal subject (e.g., a mouse subject). In some embodiments, when a composition comprising a biomaterial and a therapeutic agent (e.g., a p38 MAPK inhibitor) is implanted at a target site (e.g., mammary fat pad) of an animal subject (e.g., a mouse subject), less than or equal to 70% or lower (including, e.g., less than or equal to 60%, less than or equal to 50%, less than 40%, less than 30% or lower) of the therapeutic agent is released in vivo 8 hours after the implantation.

### BRIEF DESCRIPTION OF THE DRAWINGS

FIG. 1 is a Kaplan-Meier curve of female BALB/cJ mice inoculated orthotopically with 4T1-Luc2 cells whose tumors were surgically resected followed by implantation of exemplary drug delivery devices comprising a hydrogel (e.g., a crosslinked hyaluronic acid hydrogel) without a p38 MAPK inhibitor or exemplary drug delivery devices comprising a hydrogel (e.g., a crosslinked hyaluronic acid) and a p38 MAP kinase inhibitor (e.g., losmapimod).

FIG. 2 is a Kaplan-Meier curve of female BALB/cJ mice inoculated orthotopically with 4T1-Luc2 cells whose tumors were surgically resected followed by implantation of exemplary drug delivery devices comprising a hydrogel (e.g., a crosslinked hyaluronic acid hydrogel) without an anti-IL-1β antibody or exemplary drug delivery devices comprising a hydrogel (e.g., a crosslinked hyaluronic acid) and anti-IL-1β antibody (e.g., clone B122).

FIG. 3 is a Kaplan-Meier curve of female BALB/cJ mice inoculated orthotopically with 4T1-Luc2 cells whose tumors were surgically resected followed by implantation of exemplary drug delivery devices comprising a hydrogel (e.g., a crosslinked hyaluronic acid hydrogel) without an anti-IL-6 antibody or exemplary drug delivery devices comprising a hydrogel (e.g., a crosslinked hyaluronic acid) and an anti-IL-6 antibody (e.g., clone MP5-20F3).

FIG. 4 is a schematic representation showing interrelationships of certain proinflammatory pathways that involve p38 mitogen-activated protein kinase (MAPK) and COX-2. See Desai et al., “Mechanisms of Phytonutrient Modulation of Cyclooxygenase-2 (COX-2) and Inflammation Related to Cancer” Nutrition and Cancer, 70: 350-375 (2018). Those skilled in the art, familiar with such pathways, will be aware that proinflammatory signals such as, e.g., cytokines TNF-α, IL-6, and/or IL-1β, can stimulate COX-2 transcription via, for example, activation of a MAPK pathway. For example, IL-1β has been established to upregulate COX-2 expression through activation of a p38 MAPK pathway (Huang et al., “MAPK/ERK signal pathway involved expression of COX-2 and VEGF by IL-1beta induced in human endometriosis stomal cells in vitro” Int J Clin Exp Pathol, 6: 2129-2136 (2013) and Di Mari et al., “HETEs enhance IL-1-mediated COX-2 expression via augmentation of message stability in human colonic myofibroblasts” Am J Physiol-Gastrointest Liver Physiol., 293: 2092-2101 (2007)).

FIG. 5 is a schematic representation showing that certain non-steroidal anti-inflammatory drugs (NSAIDs) may act as inhibitors of cyclooxygenase (COX), including, e.g., inhibitors of COX-1 and/or inhibitors of COX-2, and/or as inhibitors of a p38 MAPK. See Esposito et al., “Non-steroidal anti-inflammatory drugs in Parkinson's disease” Experimental Neurology 205: 295-312 (2007). For example, in some embodiments, a NSAID may inhibit or reduce activity and/or level of COX-1 and/or COX-2. In some embodiments, a NSAID may inhibit or reduce activation of a p38 MAPK pathway or component(s) thereof, thereby reducing or inhibiting AP-1 activation.

FIG. 6 is a schematic representation of interrelationships of certain proinflammatory pathways that involve p38 mitogen-activated protein kinase (MAPK) and Wnt-β-catenin. See Bikkavilli et al., “p38 mitogen-activated protein kinase regulates canonical Wnt-β-catenin signaling by inactivation of GSK3β” Journal of Cell Science, 121: 3598-3607 (2008). Those skilled in the art, familiar with such pathways, will be aware that p38 MAPK can be activated upon Wnt3a stimulation and such stimulation can be dependent on both G-protein and Dishevelleds. p38 MAPK specific inhibitors can reduce Wnt3a-induced β-catenin expression. Thus, p38 MAPK plays a role in Wnt-β-catenin signaling, for example, by inactiving GSK3β and/or by operating downstream of Dishevelleds.

### DETAILED DESCRIPTION OF CERTAIN EMBODIMENTS

Provided herein are drug delivery compositions and devices that can localize delivery of one or more immunomodulatory agents to a target site (e.g., a site at which a tumor has been removed and/or cancer cells have been treated or killed, e.g., by chemotherapy or radiation) and thereby concentrate the action of the immunomodulatory agents to a target site in need thereof. Such drug delivery systems can be particularly useful for treating cancer. The drug delivery compositions and devices may comprise a biomaterial and an inhibitor of a proinflammatory pathway. In some aspects, the drug delivery compositions and devices may comprise a biomaterial and an inhibitor of a proinflammatory immune response mediated by a p38 mitogen-activated protein kinase (MAPK) pathway (e.g., a p38 MAPK inhibitor). The drug delivery compositions and devices may comprise a biomaterial, an inhibitor of a proinflammatory immune response mediated by a p38 mitogen-activated protein kinase (MAPK) pathway, and an activator of innate immune response. The drug delivery compositions and devices may comprise a biomaterial, an inhibitor of a proinflammatory immune response mediated by a p38 mitogen-activated protein kinase (MAPK) pathway, an activator of innate immune response, and a cytokine. The drug delivery compositions and devices may comprise a biomaterial, an inhibitor of a proinflammatory immune response mediated by a p38 mitogen-activated protein kinase (MAPK) pathway, an activator of innate immune response, and a chemokine. The drug delivery compositions and devices may further comprise one or more activators of adaptive immune response. The drug delivery compositions and devices may further comprise additional therapeutic agents (e.g., an inhibitor of a proinflammatory pathway, a modulator of macrophage effector function or chemotherapeutic agents).

In some embodiments, therapeutic agents (e.g., immunomodulatory agents) provided within drug delivery compositions and devices may mediate inflammation (e.g., chronic inflammation) induced, e.g., by surgery such as surgical tumor resection, thus providing unique tools for the treatment of cancer, particularly solid tumors. In some embodiments, therapeutic agents (e.g., immunomodulatory agents) provided within drug delivery compositions and devices may inhibit inflammation (e.g., chronic inflammation) induced, e.g., by surgery such as surgical tumor resection. In some embodiments, therapeutic agents (e.g., immunomodulatory agents) provided within drug delivery compositions and devices may reduce or inhibit activity of myeloid-derived suppressor cells (MDSCs). In some embodiments, therapeutic agents (e.g., immunomodulatory agents) provided within drug delivery compositions and devices may reduce or inhibit recruitment of immunosuppressive cells. In some embodiments, therapeutic agents (e.g., immunomodulatory agents) provided within drug delivery compositions and devices may reduce or inhibit acute inflammation. In some embodiments, drug delivery compositions and devices may further comprise one or more therapeutic agents (e.g., immunomodulatory agents) that activate the innate immune response system and/or the adaptive immune response system. Compositions, devices, methods, systems, and kits provided herein are also advantageous over existing methods in that they do not require administration of cells (e.g., adoptive cell transfer) or incorporation or presence of additional components such as microparticles, peptides, or tumor antigens.

Drug delivery compositions and devices described herein are useful for treating cancer (e.g., solid tumors) in the perioperative setting. In some embodiments, compositions and devices may deliver immunotherapies by implantation of the device or devices at the site of therapeutic need in a subject in need thereof. Drug delivery compositions and devices described herein are particularly advantageous over existing immunotherapies because, in some embodiments, they can release an immunomodulatory agent (e.g., a p38 MAPK inhibitor) directly to a site of tumor resection, avoiding systemic administration. Accordingly, drug delivery compositions and devices described herein provide a vehicle for drug delivery at the site of tumor resection that avoids potential toxicities that can be associated with traditional systemic administration of immunotherapies. Concentrating the immunotherapy at the site of tumor resection can similarly improve efficacy. In certain embodiments, the drug delivery compositions and devices are useful for slowing and/or impeding tumor growth, preventing cancer recurrence, preventing tumor metastasis, and/or preventing primary tumor regrowth.

Among other things, in some embodiments, the present disclosure provides technologies for suppression of immune responses that themselves foster additional immunosuppression (e.g., activity of MDSCs).

Without wishing to be bound by any particular theory, the present disclosure notes that, in some embodiments, technologies provided herein may reduce a type of inflammation that is generally observed in the context of chronic inflammation (e.g., as is often associated with an autoimmune disease) but, as described herein, may be activated in an acute setting (i.e., post-surgery). The present disclosure provides the insight that therapy targeting p38 as described herein may be uniquely useful in the post-tumor resection context. For example, p38 has been described in association with certain autoimmune conditions, and has been targeted in therapy for treatment of such conditions. Those skilled in the art will appreciate that many therapeutic strategies designed and/or effective to treat autoimmune disease would be disastrous in the setting of tumor resection, as they would result in worsening of the tumor progression phenotype. The present disclosure teaches that, notwithstanding this general principle, targeting p38 as described herein is surprisingly useful in the cancer therapy context.

In some embodiments, described therapy targeting p38 may be combined, for example, with therapies that include other immune modulation strategies such as, for example, activation/agonism of innate immune system (e.g., via administration of an agent such as a STING agonist or a TLR agonist).

### Drug Delivery Compositions and Devices

### Biomaterial (e.g., Hydrogel)

Drug delivery compositions and devices include a biomaterial. In certain embodiments, the biomaterial is a scaffold or depot. The scaffold or depot comprises any synthetic or naturally occurring material that is suitable for containing and promoting the sustained or extended release of any therapeutic agents in the drug delivery compositions and devices as described herein. Accordingly, a biomaterial possesses properties that provide the advantageous properties of the compositions and devices described herein (e.g., storage modulus, biodegradation, release profile of therapeutic agents).

In certain embodiments, a biomaterial extends the release of a therapeutic agent in the tumor resection site relative to administration of the same therapeutic agent in solution. In certain embodiments, a biomaterial extends the release of a therapeutic agent in the tumor resection site relative to administration of the same therapeutic agent in solution by at least 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 18 hours, 24 hours, 2 days, 3 days, 4 days, 5 days, 6 days, 7 days, 2 weeks, 3 weeks, or 4 weeks.

In some embodiments, a biomaterial extends release of a therapeutic agent (e.g., a p38 MAPK inhibitor) so that, when assessed at a specified time point after administration, more therapeutic agent is present in the tumor resection site than that is observed when the therapeutic agent is administered in solution. For example, in some embodiments, when assessed at 24 hours after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution. In some embodiments, when assessed at 48 hours after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution. In some embodiments, when assessed at 3 days after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution. In some embodiments, when assessed at 5 days after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution.

In some embodiments, a biomaterial is characterized by a storage modulus of at least 500 Pa, at least 1000 Pa, at least 1500 Pa, at least 2000 Pa, at least 2500 Pa, at least 3000 Pa, at least 4000 Pa, at least 5000 Pa, at least 10 kPa, at least 15 kPa, or higher. In some embodiments a biomaterial is characterized by a storage modulus of no more than 50 kPa, no more than 40 kPa, no more than 30 kPa, no more than 20 kPa, no more than 10 kPa, no more than 5000 kPa, no more than 4000 Pa, no more than 3000 Pa, no more than 2000 Pa, or lower. Combinations of the above-mentioned ranges are also possible. For example, in some embodiments, a biomaterial is characterized by a storage modulus of 500 Pa to 50,000 Pa, or 1000 Pa to 20 kPa, or 1000 Pa to 10 kPa, or 1000 Pa to 5000 Pa, or 1000 Pa to 3000 Pa.

In certain embodiments, the biomaterial comprises hyaluronic acid, alginate, chitosan, chitin, chondroitin sulfate, dextran, gelatin, collagen, starch, cellulose, polysaccharide, fibrin, ethylene-vinyl acetate (EVA), poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyphosphazene, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, derivatives thereof, and/or combinations thereof.

In certain embodiments, the biomaterial is or comprises a non-crosslinked biomaterial. In certain embodiments, the biomaterial is or comprises a crosslinked biomaterial. For example, in some embodiments, such a crosslinked biomaterial is or comprises a hydrogel. Hydrogels can be crosslinked using any methods known in the art. Those skilled in the art will appreciate that, in some cases, hydrogels can be crosslinked, for example, using chemical crosslinking methods (e.g., by using a small-molecule cross-linker, which can be derived from a natural source or synthesized), polyelectrolyte crosslinking (e.g., mixing a polymer with a second polymer comprising an opposite charge), thermal-induced crosslinking, photo-induced crosslinking (e.g., using vinyl sulfone, methacrylate, acrylic acid), pH-induced crosslinking, and/or enzyme-catalyzed crosslinking. In some embodiments, one or more cross-linking methods described in Parhi, Adv Pharm Bull., Review 7(4): 515-530 (2017) can be used in forming a hydrogel. In some embodiments, a hydrogel can be cross-linked by attaching thiols (e.g., EXTRACEL®, HYSTEM®), methacrylates, hexadecylamides (e.g., HYMOVIS®), and/or tyramines (e.g., CORGEL®). In some embodiments, a hydrogel can be crosslinked directly with formaldehyde (e.g., HYLAN-A®), divinylsulfone (DVS) (e.g., HYLAN-B®), 1,4-butanediol diglycidyl ether (BDDE) (e.g., RESTYLANE®), glutaraldehyde, and/or genipin (see, e.g., Khunmanee et al. “Crosslinking method of hyaluronic-based hydrogel for biomedical applications” J Tissue Eng. 8: 1-16 (2017)). In some embodiments, a hydrogel is crosslinked with divinylsulfone (DVS) (e.g., HYLAN-B®).

In some embodiments, a hydrogel biomaterial is characterized by a storage modulus of at least 500 Pa, at least 1000 Pa, at least 1500 Pa, at least 2000 Pa, at least 2500 Pa, at least 3000 Pa, at least 4000 Pa, at least 5000 Pa, at least 10 kPa, at least 15 kPa, at least 20 kPa, at least 25 kPa, at least 30 kPa, at least 35 kPa, at least 40 kPa, or higher. In some embodiments, a hydrogel biomaterial is characterized by a storage modulus of no more than 50 kPa, no more than 40 kPa, no more than 30 kPa, no more than 20 kPa, no more than 10 kPa, no more than 5000 Pa, no more than 4000 Pa, no more than 3000 Pa, no more than 2000 Pa, or lower. Combinations of the above-mentioned ranges are also possible. For example, in some embodiments, a hydrogel biomaterial is characterized by a storage modulus of 500 Pa to 50 kPa, or 1000 Pa to 50 kPa, or 1000 Pa to 20 kPa, or 1000 Pa to 10 kPa, or 500 Pa to 5000 Pa, or 500 Pa to 3000 Pa. In some embodiments, the storage modulus of a hydrogel biomaterial may be determined when it is fully saturated with an aqueous solution (e.g., water).

In some embodiments, a biomaterial (e.g., a hydrogel biomaterial) is characterized by a viscosity (e.g., measured at 10° C. with a shear rate of 1000 s⁻¹) of at least 5 mPa/s, at least 10 mPa/s, at least 20 mPa/s, at least 30 mPa/s, at least 40 mPa/s, at least 50 mPa/s, or higher. In some embodiments, a hydrogel biomaterial is characterized by a viscosity (e.g., measured at 10° C. with a shear rate of 1000 s⁻¹) of no more than 50 mPa/s, no more than 45 mPa/s, no more than 40 mPa/s, no more than 35 mPa/s, no more than 30 mPa/s, no more than 25 mPa/s, no more than 20 mPa/s, no more 15 mPa/s, no more than 10 mPa/s, or lower. Combinations of the above-mentioned ranges are also possible. For example, in some embodiments, a hydrogel biomaterial is characterized by a viscosity (e.g., measured at 10° C. with a shear rate of 1000 s⁻¹) of 5-50 mPa/s, or 10-40 mPa/s, or 20-30 mPa/s. In some embodiments, viscosity of a hydrogel biomaterial can be measured using a rheometer.

In certain embodiments, a biomaterial (e.g., a hydrogel biomaterial) is or comprises hyaluronic acid, alginate, chitosan, chondroitin sulfate, dextran, gelatin, collagen, starch, cellulose, polysaccharide, fibrin, polyethylene glycol (PEG), PEG diacrylate (PEGDA), disulfide-containing PEGDA (PEGSSDA), PEG dimethacrylate (PEGDMA), poly(2-hydroxyethyl methacrylate) (pHEMA), poly(beta-amino ester) (PBAE), poly(aspartic acid), poly(glutamic acid), poly(propylene glycol) (PPG), poly(vinyl alcohol) (PVA), polyacetal, polyglycerol, polyglucuronic acid, or combinations thereof. In certain embodiments, when the biomaterial is a hydrogel, then the therapeutic agent(s) of the composition or device are hydrophilic molecules. In certain embodiments, when the biomaterial is a hydrogel, then the therapeutic agent(s) of the composition or device are hydrophobic molecules. In certain embodiments, when the biomaterial is a hydrogel, then the therapeutic agent(s) of the composition or device are hydrophobic or hydrophilic molecules. In certain embodiments, when the biomaterial is a hydrogel, then the therapeutic agent(s) of the composition or device are hydrophobic and hydrophilic molecules.

In certain embodiments, the biomaterial is hyaluronic acid or alginate. In certain embodiments, the biomaterial is cross-linked hyaluronic acid or cross-linked alginate. In certain embodiments, the biomaterial comprises hyaluronic acid or alginate. In certain embodiments, the biomaterial comprises cross-linked hyaluronic acid or cross-linked alginate. In certain embodiments, the hydrogel is hyaluronic acid or alginate. In certain embodiments, the hydrogel is cross-linked hyaluronic acid or cross-linked alginate. In certain embodiments, the hydrogel comprises hyaluronic acid or alginate. In certain embodiments, the hydrogel comprises cross-linked hyaluronic acid or cross-linked alginate.

In certain embodiments, the biomaterial comprises hyaluronic acid. In certain embodiments, the biomaterial comprises cross-linked hyaluronic acid. In certain embodiments, the biomaterial is hyaluronic acid. In certain embodiments, the biomaterial is cross-linked hyaluronic acid. In certain embodiments, the hydrogel comprises hyaluronic acid. In certain embodiments, the hydrogel comprises cross-linked hyaluronic acid. In certain embodiments, the hydrogel is hyaluronic acid. In certain embodiments, the hydrogel is cross-linked hyaluronic acid.

Hyaluronic acid, also known as hyaluronan, is an anionic, non-sulfated glycosaminoglycan distributed widely throughout connective, epithelial, and neural tissues. It is unique among glycosaminoglycans in that it is non-sulfated, forms in the plasma membrane instead of the Golgi, and can be very large, with its molecular weight often reaching the millions.

One of the chief components of the extracellular matrix, hyaluronic acid plays a significant role in cancer metastasis as it contributes significantly to cell proliferation and migration. In some cancers, hyaluronic acid levels correlate with malignancy and poor prognosis. Hyaluronic acid is often used as a tumor marker for certain cancers (e.g., prostate and breast cancer) and may also be used to monitor the progression of the disease in individuals. Therefore, use of hyaluronic acid as a biomaterial in the disclosed drug delivery compositions and devices provides an unexpectedly useful and efficacious cancer therapy.

In certain embodiments, hyaluronic acid can be cross-linked by attaching thiols (e.g., EXTRACEL®, HYSTEM®), methacrylates, hexadecylamides (e.g., HYMOVIS®), and tyramines (e.g., CORGEL®). Hyaluronic acid can also be cross-linked directly with formaldehyde (e.g., HYLAN-A®), divinylsulfone (DVS) (e.g., HYLAN-B®), 1,4-butanediol diglycidyl ether (BDDE) (e.g., RESTYLANE®), glutaraldehyde, or genipin (see, e.g., Khunmanee et al., “Crosslinking method of hyaluronic-based hydrogel for biomedical applications” J Tissue Eng. 8: 1-16 (2017)). In some embodiments, hyaluronic acid is crosslinked with divinylsulfone (DVS) (e.g., HYLAN-B®).

In certain embodiments, hyaluronic acid comprises thiol-modified hyaluronic acid and a cross-linking agent. In certain embodiments, the hydrogel comprises thiol-modified hyaluronic acid (e.g., GLYCOSIL®), and a thiol-reactive PEGDA cross-linker (e.g., EXTRALINK®). In certain embodiments, the thiol-modified hyaluronic acid and the thiol-reactive PEGDA cross-linker are combined to form a cross-linked hydrogel useful in the drug delivery compositions and devices described herein.

In certain embodiments, the amount and concentration of thiol-modified hyaluronic acid, thiol-reactive hyaluronic acid, and cross-linking agent can be adjusted to provide drug delivery compositions and devices with desired physical properties, such as having a storage modulus of about 500 Pa to about 3000 Pa.

In certain embodiments, the biomaterial comprises alginate. In certain embodiments, the biomaterial comprises cross-linked alginate. In certain embodiments, the biomaterial is alginate. In certain embodiments, the biomaterial is cross-linked alginate. In certain embodiments, the hydrogel comprises alginate. In certain embodiments, the hydrogel comprises cross-linked alginate. In certain embodiments, the hydrogel is alginate. In certain embodiments, the hydrogel is cross-linked alginate. In certain embodiments, the biomaterial does not comprise alginate. In certain embodiments, the biomaterial is not alginate. In certain embodiments, the hydrogel is not alginate. In certain embodiments, the hydrogel does not comprise alginate.

In certain embodiments, alginate can be cross-linked ionically by adding a salt that promotes cross-linking (e.g., calcium chloride).

In certain embodiments, alginate comprises alginate and a cross-linking agent (e.g., calcium chloride). In certain embodiments, the hydrogel comprises alginate and a cross-linking agent (e.g., calcium chloride). In certain embodiments, the alginate and the calcium chloride (e.g., ionic cross-linker) are combined to form a cross-linked hydrogel useful in the drug delivery compositions and devices described herein.

In certain embodiments, the amount and concentration of alginate and calcium chloride can be adjusted to provide drug delivery compositions and devices with desired physical properties, such as having a storage modulus of about 500 Pa to about 3000 Pa.

In certain embodiments, the biomaterial is a hydrophobic polymer. In certain embodiments, the hydrophobic polymer is ethylene-vinyl acetate (EVA), poly(lactic-co-glycolic) acid (PLGA), polylactic acid (PLA), polyglycolic acid (PGA), polydioxanone (PDO), polyhydroxybutyrate (PHB), polycaprolactone (PCL), poly(ester amide), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyphosphazene, polyanhydride, poly(N-vinyl-2-pyrrolidone) (PVP), poly(acrylic acid) (PAA), poly(methacrylic acid) (PMA), poly(alpha ester), poly(ortho ester), polyphosphoester, polyurethane, polycarbonate, polyamide, or polyhydroxyalkanoate. Use of a hydrophobic polymer as the biomaterial may be particularly useful when the therapeutic agent(s) in the composition or device is hydrophilic. Hydrophobic therapeutic agents would be expected to be released over longer periods of time (e.g., days/weeks) rather than a release timescale more conducive to imparting a therapeutic effect (e.g., hours). Accordingly, in certain embodiments, when the biomaterial is a hydrophobic polymer, then the therapeutic agent(s) of the composition or device are hydrophilic molecules.

In certain embodiments, the biomaterial comprises a cross-linked biologic. In certain embodiments, the biologic is cross-linked by the self-immolating cross-linker dithio-bis(ethyl 1H-imidazole-1-carboxylate) (DIC). In certain embodiments, the resultant hydrogel is loaded with a small molecule.

### Inhibitors of Proinflammatory Pathways

The drug delivery compositions and devices may comprise an inhibitor of a proinflammatory pathway. The drug delivery compositions and devices may comprise more than one inhibitor of a proinflammatory pathway. In some embodiments, an inhibitor of a proinflammatory pathway may prevent recruitment of immunosuppressive cells. In some embodiments, an inhibitor of a proinflammatory pathway may prevent acute inflammation. In some embodiments, an inhibitor of a proinflammatory pathway may inhibit, for example, an immune response that induces inflammation, including, e.g., production of one or more proinflammatory cytokines (e.g., TNF-alpha, IL-1β, and/or IL-6), increased activity and/or proliferation of Th1 cells, recruitment of myeloid cells, etc. For example, in some embodiments, an inhibitor of a proinflammatory pathway can be an inhibitor of IL-1β. In some embodiments, an inhibitor of a proinflammatory pathway can be an inhibitor of IL-6.

In certain embodiments, the inhibitor of a proinflammatory pathway is an inhibitor of a proinflammatory immune response mediated by a p38 mitogen-activated protein kinase (MAPK) pathway as described herein.

In certain embodiments, the inhibitor of a proinflammatory pathway prevents recruitment of immunosuppressive cells. In certain embodiments, the inhibitor of a proinflammatory pathway is an inhibitor, antagonist, or partial agonist of CCR2, CCR5, CXCR2, CXCR4, CXCL12, or CCL2. In certain embodiments, the inhibitor of a proinflammatory pathway is an inhibitor, antagonist, or partial agonist of CCR5, CXCR2, CXCL12, or CCL2.

In certain embodiments, the inhibitor of a proinflammatory pathway is an inhibitor, antagonist, or partial agonist of CCR2. In certain embodiments, CCR2 is related to a p38 MAPK pathway (e.g., as described in Montague et al., J. Inflammation 2018, 15:101; and Xu et al., Am. J. Transl. Res. 2017, 9, 2878-2890). In certain embodiments, the inhibitor, antagonist, or partial agonist of CCR2 is PF-04136309, CCX872-B, or plozalizumab. In certain embodiments, the inhibitor of a proinflammatory pathway is PF-04136309, CCX872-B, or plozalizumab. In certain embodiments, the inhibitor of a proinflammatory pathway is not an inhibitor, antagonist, or partial agonist of CCR2. In certain embodiments, the inhibitor of a proinflammatory pathway is not PF-04136309.

In certain embodiments, the inhibitor of a proinflammatory pathway is an inhibitor, antagonist, or partial agonist of CCR5. In certain embodiments, CCR5 is related to a p38 MAPK pathway (e.g., as described in Lei, et al., Biochem. Biophys. Res. Commun. 2005, 329, 610-615; and Manes, et al., J. Exp. Med. 2003, 198, 1381-1389). In certain embodiments, the inhibitor, antagonist, or partial agonist of CCR5 is maraviroc, DAPTA, GSK706769, INCB009471, GW873140, Vicriviroc, or PRO 140. In certain embodiments, the inhibitor of a proinflammatory pathway is maraviroc, DAPTA, GSK706769, INCB009471, GW873140, Vicriviroc, or PRO 140.

In certain embodiments, the inhibitor of a proinflammatory pathway is an inhibitor, antagonist, or partial agonist of CCR2 and CCR5. In certain embodiments, the inhibitor, antagonist, or partial agonist of CCR2 and CCR5 is PF-04634817, cenicriviroc, or BMS-813160. In certain embodiments, the inhibitor of a proinflammatory pathway is PF-04634817, cenicriviroc, or BMS-813160.

In certain embodiments, the inhibitor of a proinflammatory pathway is an inhibitor, antagonist, or partial agonist of CXCR2. In certain embodiments, the inhibitor, antagonist, or partial agonist of CXCR2 is danirixin, QBM076, SX-682, or SB225002. In certain embodiments, the inhibitor of a proinflammatory pathway is danirixin, QBM076, SX-682, or SB225002.

In certain embodiments, the inhibitor of a proinflammatory pathway is an inhibitor, antagonist, or partial agonist of CXCR4. In certain embodiments, CXCR4 is related to a p38 MAPK pathway (e.g., as described in Lei, et al., Biochem. Biophys. Res. Commun. 2005, 329, 610-615; and Trushin, et al., J. Immunol. 2007, 178, 4846-4853). In certain embodiments, the inhibitor, antagonist, or partial agonist of CXCR4 is plerixafor, AMD070, AMD3465, AMD11070, LY2510924, MSX-122, TG-0054, CX-01, X4P-001, BL-8040, USL311, or SP01A. In certain embodiments, the inhibitor of a proinflammatory pathway is plerixafor, AMD070, AMD3465, AMD11070, LY2510924, MSX-122, TG-0054, CX-01, X4P-001, BL-8040, USL311, or SP01A. In certain embodiments, the inhibitor of a proinflammatory pathway is not an inhibitor, antagonist, or partial agonist of CXCR4.

In certain embodiments, the inhibitor of a proinflammatory pathway is an inhibitor, antagonist, or partial agonist of CXCL12. In certain embodiments, CXCL12 is related to a p38 MAPK pathway (e.g., as described in Gao, et al., Int. J. Clin. Exp. Pathol. 2018, 11, 3119-3125).

In certain embodiments, the inhibitor of a proinflammatory pathway is an inhibitor, antagonist, or partial agonist of CCL2. In certain embodiments, CCL2 is related to a p38 MAPK pathway (e.g., as described in Cho, et al., J. Neuroimmunol. 2008, 199, 94-103; and Marra, et al., Am. J. Physiol. Gastrointest. Liver Physiol. 2004, 287, G18-26). In certain embodiments, the inhibitor, antagonist, or partial agonist of CCL2 is bindarit.

In certain embodiments, the inhibitor of a proinflammatory pathway is PF-04136309, CCX872-B, plozalizumab, maraviroc, DAPTA, GSK706769, INCB009471, GW873140, Vicriviroc, PRO 140, PF-04634817, cenicriviroc, BMS-813160, danirixin, QBM076, SX-682, SB225002, plerixafor, AMD070, AMD3465, AMD11070, LY2510924, MSX-122, TG-0054, CX-01, X4P-001, BL-8040, USL311, or SP01A.

In certain embodiments, the inhibitor of a proinflammatory pathway is CCX872-B, plozalizumab, maraviroc, DAPTA, GSK706769, INCB009471, GW873140, Vicriviroc, PRO 140, PF-04634817, cenicriviroc, BMS-813160, danirixin, QBM076, SX-682, SB225002, plerixafor, AMD070, AMD3465, AMD11070, LY2510924, MSX-122, TG-0054, CX-01, X4P-001, BL-8040, USL311, or SP01A.

In certain embodiments, the inhibitor of a proinflammatory pathway prevents acute inflammation. In certain embodiments, the inhibitor of a proinflammatory pathway is an anti-IL-1α antibody, an anti-IL-1β antibody, an anti-IL-1R antibody, an IL-1 inhibitor, an anti-IL-6 antibody, an anti-IL-6R antibody, an anti-IL17 antibody, an anti-IL-17A antibody, an anti-IL-17RA antibody, an anti-IL-23/IL-12 antibody, or an anti-IL-23 antibody.

In certain embodiments, the inhibitor of a proinflammatory pathway is an anti-IL-1α antibody. In certain embodiments, the anti-IL-1α antibody is MABp1. In certain embodiments, the inhibitor of a proinflammatory pathway is MABp1.

In certain embodiments, the inhibitor of a proinflammatory pathway is an anti-IL-1β antibody. In certain embodiments, IL-1β is related to a p38 MAPK pathway (e.g., as described in Kulawik, et al., J. Biol. Chem. 2017, 292, 6291-6302; Rovin, et al., Cytokine 1999, 11, 118-126; Laporte, et al., Am. J. Physiol. Lung Cell Mol. Physiol. 2000, 279, L932-L941; Baldassare, et al., J. Immunol. 1999, 162, 5367-5373; and Weber, et al. Sci. Signal. 2010, 3, cm1). In certain embodiments, the anti-IL-1β antibody is canakinumab. In certain embodiments, the inhibitor of a proinflammatory pathway is canakinumab.

In certain embodiments, the inhibitor of a proinflammatory pathway is an anti-IL-1R antibody. In certain embodiments, IL-1R is related to a p38 MAPK pathway (e.g., as described in Weber, et al., Sci. Signal. 2010, 3, cm1; and Jain, et al., Nat. Commun. 2018, 9:3185). In certain embodiments, the anti-IL-1R antibody is anakinra. In certain embodiments, the inhibitor of a proinflammatory pathway is anakinra.

In certain embodiments, the inhibitor of a proinflammatory pathway is an IL-1 inhibitor. In certain embodiments, the IL-1 inhibitor is rilonacept. In certain embodiments, the inhibitor of a proinflammatory pathway is rilonacept.

In certain embodiments, the inhibitor of a proinflammatory pathway is an anti-IL-6 antibody. In certain embodiments, IL-6 is related to a p38 MAPK pathway (e.g., as described in Sinfield, et al., Biochem. Biophys. Res. Commun. 2013, 430, 419-424; Suzuki, et al., FEBS Lett. 2000, 465, 23-27; and Nishikai-Yan Shen, et al., PLoS One 2017, 12, 1-17). In certain embodiments, the anti-IL-6 antibody is olokizumab, clazakizumab, OPR-003, sirukumab, ARGX-109, FE301, or FM101. In certain embodiments, the inhibitor of a proinflammatory pathway is olokizumab, clazakizumab, OPR-003, sirukumab, ARGX-109, FE301, or FM101.

In certain embodiments, the inhibitor of a proinflammatory pathway is an anti-IL-6R antibody. In certain embodiments, the anti-IL-6R antibody is tocilizumab, sarilumab, or vobarilizumab. In certain embodiments, the inhibitor of a proinflammatory pathway is tocilizumab, sarilumab, or vobarilizumab.

In certain embodiments, the inhibitor of a proinflammatory pathway is an anti-IL-17 antibody. In certain embodiments, IL-17 is related to a p38 MAPK pathway (e.g., as described in Noubade, et al., Blood 2011, 118, 3290-3300; Roussel, et al., J. Immunol. 2010, 184, 4531-4537; and Mai, et al., J. Biol. Chem. 2016, 291, 4939-4954). In certain embodiments, the anti-IL-17 antibody is ixekizumab, bimekizumab, ALX-0761, CJM112, CNTO 6785, LY3074828, SCH-900117, or MSB0010841. In certain embodiments, the inhibitor of a proinflammatory pathway is ixekizumab, bimekizumab, ALX-0761, CJM112, CNTO 6785, LY3074828, SCH-900117, or MSB0010841.

In certain embodiments, the inhibitor of a proinflammatory pathway is an anti-IL-17A antibody. In certain embodiments, the anti-IL17A antibody is secukinumab. In certain embodiments, the inhibitor of a proinflammatory pathway is secukinumab.

In certain embodiments, the inhibitor of a proinflammatory pathway is an anti-IL-17RA antibody. In certain embodiments, the anti-IL17RA antibody is brodalumab. In certain embodiments, the inhibitor of a proinflammatory pathway is brodalumab.

In certain embodiments, the inhibitor of a proinflammatory pathway is an anti-IL-23/IL-12 antibody. In certain embodiments, the anti-IL-23/IL-12 antibody is ustekinumab or briakinumab. In certain embodiments, the inhibitor of a proinflammatory pathway is ustekinumab or briakinumab.

In certain embodiments, the inhibitor of a proinflammatory pathway is an anti-IL-23 antibody. In certain embodiments, IL-23 is related to a p38 MAPK pathway (e.g., as described in Tang, et al., Immunology 2012, 135, 112-124; and Canavese, et al., J. Clin. Exp. Dermatol. Res. 2011, S2:002. doi:10.4172/2155-9554). In certain embodiments, the anti-IL-23 antibody is tildrakizumab, BI 655066, or guselkumab. In certain embodiments, the inhibitor of a proinflammatory pathway is tildrakizumab, BI 655066, or guselkumab.

In certain embodiments, the inhibitor of a proinflammatory pathway is MABp1, canakinumab, anakinra, rilonacept, olokizumab, clazakizumab, OPR-003, sirukumab, ARGX-109, FE301, FM101, tocilizumab, sarilumab, vobarilizumab, ixekizumab, bimekizumab, ALX-0761, CJM112, CNTO 6785, LY3074828, SCH-900117, MSB0010841, secukinumab, brodalumab, ustekinumab briakinumab, tildrakizumab, BI 655066, or guselkumab.

In certain embodiments, the inhibitor of a proinflammatory pathway is a TGFβR inhibitor. In certain embodiments, TGFβR is related to a p38 MAPK pathway (e.g., as described in Yu et al., EMBO J. 2002, 21, 3749-3759; Sato, et al., J. Invest. Dermatol. 2002, 118, 704-711; and Hanafusa, et al., J. Biol. Chem. 1999, 274, 27161-27167). In certain embodiments, the TGFβR inhibitor is galunisertib. In certain embodiments, the inhibitor of a proinflammatory pathway is galunisertib.

In certain embodiments, an inhibitor of a proinflammatory pathway is or comprises a specialized pro-resolving mediator (SPM). SPMs are long-chain fatty acid-derived lipid mediators, which are involved in a coordinated resolution program to prevent excessive inflammation and/or to resolve acute inflammatory response. Examples of such SPMs include, e.g., arachidonic acid (AA)-derived lipoxins and docosahexaenoic acid (DHA)-derived resolvins. Resolution is an active process involving the production of molecules that signal through specific cell-surface receptors to temper inflammation, enhance efferocytosis, and repair tissue damage without compromising host defense. See, e.g., Cai et al., “MerTK cleavage limits proresolving mediator biosynthesis and exacerbates tissue inflammation” PNAS, 113: 6526-6531 (2016); and Serhan et al., “Novel anti-inflammatory—Pro-resolving mediators and their receptors” Curr Top Med Chem 11: 629-647 (2011). Cyclooxygenase enzymes (e.g., COX-2) may be involved in production of certain SPMs.

In some embodiments, a SPM that may be useful as an inhibitor of a proinflammatory pathway is or comprises a resolvin. Resolvins were shown to enhance clearance of tumor cell debris via macrophage phagocytosis and counterregulate release of cytokines/chemokines, including, e.g., TNFα, IL-6, IL-8, CCL4, and/or CCL5. See, e.g., Sulciner et al., “Resolvins suppress tumor growth and enhance cancer therapy” J Exp Med 215: 115-140 (2018). Resolvins (Rvs) are divided into several classes: resolvin Ds (RvDs) derived from decosahexaenoic acid (DHA); resolvin Es (RvEs) derived from eicosapentaenoic acid (EPA); resolvin D_{n-6DPA} (RvD_{n-6DPA}) derived from DPA isomer, osbonic acid; resolvin D_{n-3DPA} (RvD_{n-3DPA}) derived from DPA isomer, clupanodonic acid; resolvin Ts (RvTs) derived from clupanodonic acid that, in contrast to RvD_{n-3DPA} (possessing a 17S-hydroxyl residue), possesses a 17R-hydroxyl residue. One of those skilled in the art will appreciate that, in some cases, a resolvin may be or comprise RvD1, RvD2, RvD3, RvD4, RvD5, RvD6, 17R-RvD1, 17R-RvD2, 17R-RvD3, 17R-RvD4, 17R-RvD5, 17R-RvD6, RvE1, 18S-RvE1, RvE2, RvE3, RvT1, RvT2, RvT3, RvT4, RvD1ₙ₋₃, RvD2ₙ₋₃, RvD5ₙ₋₃, or combinations thereof.

In some embodiments, a SPM that may be useful as an inhibitor of a proinflammatory pathway is or comprises a lipoxin (including, e.g., LxA4, LxB4, 15-epi-LxA4, and/or 15-epi-LxB4), a protectin/neuroprotectin (e.g., DHA-derived protectins/neuroprotectins and/or n-3 DPA-derived protectins/neuroprotectins), maresins (e.g., DHA-derived maresins and/or n-3 DPA-derived maresins), and other DPA metabolites.

### Inhibitors of a Proinflammatory Immune Response Mediated by a p38 Mitogen-Activated Protein Kinase (MAPK) Pathway

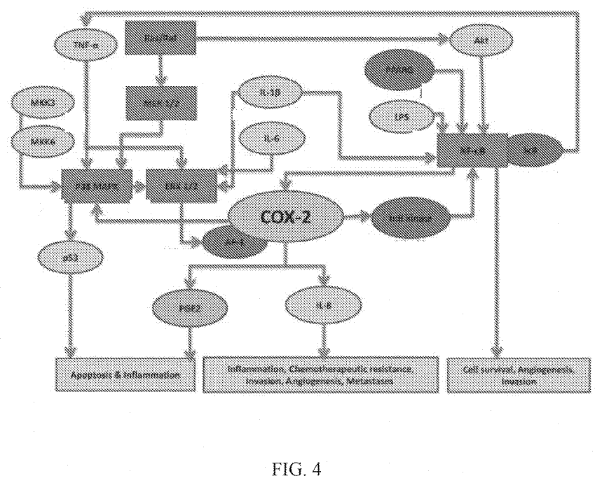

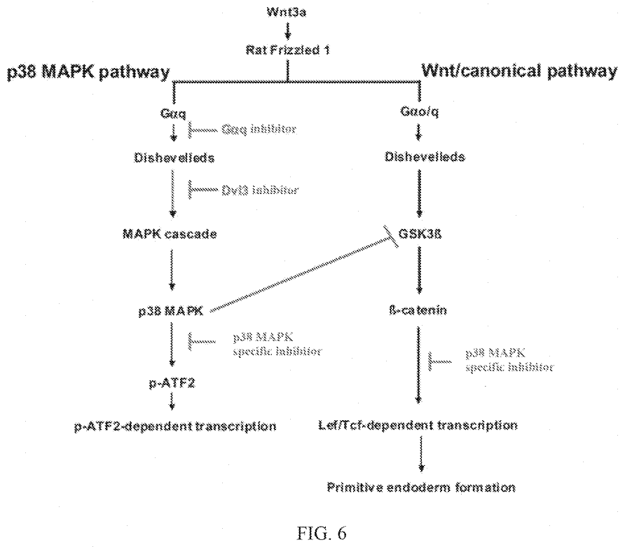

The present disclosure recognizes, among other things, that inhibiting proinflammatory immune response mediated by a p38 mitogen-activated protein kinase (MAPK) pathway (e.g., by administration of a p38 MAPK inhibitor to modulate (e.g., inhibit) a p38 MAPK-mediated proinflammatory pathway or component(s) thereof; see, for example, FIGS. 4-6) at a target site (e.g., a tumor resection site) can reduce the risk of cancer recurrence and thus prolong survival. It is unexpected that inhibition of MAPK can promote antitumor immunity since MAPK-targeted therapy (e.g., inhibition of the BRAF/MEK/ERK module) was reported to induce transcriptional signatures associated with resistance to anti-PD-1 immune checkpoint blockade therapy, which may in turn negatively impact responsiveness to anti-PD-1/L1 cancer therapy (See, e.g., Hugo et al., Cell 2016, 165, 35-44).

Accordingly, in some embodiments, provided herein are drug delivery compositions and devices comprising an inhibitor of a proinflammatory immune response mediated by a p38 mitogen-activated protein kinase (MAPK) pathway. In some embodiments, drug delivery compositions and devices provided herein may comprise more than one inhibitor of a proinflammatory immune response mediated by a p38 mitogen-activated protein kinase (MAPK) pathway.

The p38 family of MAPKs includes the p38α, p38β, p38γ, and p38δ isoforms. p38 MAPK is activated by a large number of immune receptors, thus inhibition of a signaling module or a regulatory target that functions either upstream or downstream of p38 may provide an efficacious and selective method of inhibiting the molecular pathway and the proinflammatory immune response it mediates.

For example, p38 MAPK may be activated by mitogen-activated protein kinase kinase 3 (MAP2K3), mitogen-activated protein kinase kinase 6 (MAP2K6), mitogen-activated protein kinase kinase kinase 1 (MAP3K1), and/or mitogen-activated protein kinase kinase kinase 4 (MAP3K4). Thus, inhibiting upstream targets of p38 MAPK may be effective in inhibiting a p38 MAPK pathway.

Inhibition of downstream targets of p38 MAPK may also be an effective means of inhibiting a p38 MAPK pathway. Downstream of p38 MAPK, for example, mitogen-activated protein kinase interacting protein kinases 1 and 2 (MNK1 and MNK2) are activated by a p38 MAPK pathway. The MNK kinases play important roles in regulating mRNA translation and, as a result, are key mediators of oncogenic progression, drug resistance, production of proinflammatory cytokines and cytokine signaling. Mitogen- and stress-activated kinase 1 and 2 (MSK1 and MSK2) are also downstream targets of p38 MAPK, and affect inflammatory responses. MAP kinase-activated protein kinase 2, 3, and 5 (MK2, MK3, MK5) are activated by p38 MAPK and are involved in cellular stress and inflammatory responses.

In view of the foregoing, inhibition of a p38 MAPK pathway may provide a therapeutic strategy for the treatment of cancer. In particular, local inflammatory wound response and systemic inflammation processes together may activate dormant micrometastases or induce the propagation of residual cancer cells, thus increasing the risk of cancer recurrence. Therefore, inhibiting the proinflammatory immune response mediated by a p38 MAPK pathway at a tumor resection site can reduce the risk of cancer recurrence and prolong survival of a subject.

In certain embodiments, the inhibitor of a proinflammatory immune response mediated by a p38 MAPK pathway is a p38 MAP kinase inhibitor. In certain embodiments, the p38 MAP kinase inhibitor is an inhibitor of p38α, p38β, p38γ, and/or p38δ MAP kinase. In certain embodiments, the p38 MAP kinase inhibitor is semapimod, pexmetinib, BMS-582949, losmapimod, pamapimod, ralimetinib, doramapimod, VX-702, VX-745, TAK-715, SB239063, SB202190, SB203580, SCIO 469, PH-797804, AZD7624, ARRY-797, ARRY-614, AVE-9940, LY3007113, skepinone-L, UM-164, SCIO 323, SX-011, SK-F860002, SB706504, SB681323, CHF-6297, RWJ-67657, Org48762-0, ML3403, JX-401, EO-1428, DBM 1285, AMG-548, AL-8697, PD-169316, PF-03715455, PH-797804, selonsertib, sorafenib, or dilmapimod. In certain embodiments, the p38 MAP kinase inhibitor comprises a quinazolinone, pyrimido-pyrimidone, pyrido-pyrimidone, pyrazole, quinolinone, and/or naphthyridinone core structure. In certain embodiments, the p38 MAP kinase inhibitor is losmapimod.

In certain embodiments, the inhibitor of a proinflammatory immune response mediated by a p38 MAPK pathway is an inhibitor of p38α, p38β, p38γ, and/or p38δ MAP kinase. In certain embodiments, the inhibitor of a proinflammatory immune response mediated by a p38 MAPK pathway is semapimod, pexmetinib, BMS-582949, losmapimod, pamapimod, ralimetinib, doramapimod, VX-702, VX-745, TAK-715, SB239063, SB202190, SB203580, SCIO 469, PH-797804, AZD7624, ARRY-797, ARRY-614, AVE-9940, LY3007113, skepinone-L, UM-164, SCIO 323, SX-011, SK-F860002, SB706504, SB681323, CHF-6297, RWJ-67657, Org48762-0, ML3403, JX-401, EO-1428, DBM 1285, AMG-548, AL-8697, PD-169316, PF-03715455, PH-797804, selonsertib, sorafenib, or dilmapimod. In certain embodiments, the inhibitor of a proinflammatory immune response mediated by a p38 MAPK pathway comprises a quinazolinone, pyrimido-pyrimidone, pyrido-pyrimidone, pyrazole, quinolinone, and/or naphthyridinone core structure. In certain embodiments, the inhibitor of a proinflammatory immune response mediated by a p38 MAPK pathway is losmapimod.

In certain embodiments, the p38 MAP kinase inhibitor binds to an ATP binding site of at least one p38 MAP kinase, e.g., p38α, p38β, p38γ, and/or p38δ MAP kinase. In certain embodiments, the p38 MAP kinase inhibitor is an allosteric inhibitor of at least one p38 MAP kinase, e.g., p38α, p38β, p38γ, and/or p38δ MAP kinase.

In certain embodiments, the inhibitor of a proinflammatory immune response mediated by a p38 MAPK pathway is an inhibitor of an upstream effector of p38 MAPK. In certain embodiments, the inhibitor of a proinflammatory immune response mediated by a p38 MAPK pathway is an inhibitor of RIPK1, RIPK2, RIPK3, RIPK4, RAC1, CDC42, MTK1, TAK1, MEKK1, MEKK2, MEKK3, MEKK4, DLK, MLK2, TAO1, TAO2, TLP2, TPL2, ASK1, MKK3, MKK4, and/or MKK6. In certain embodiments, the inhibitor of a proinflammatory immune response mediated by a p38 MAPK pathway is an inhibitor of a downstream effector of p38 MAPK. In certain embodiments, the inhibitor of a proinflammatory immune response mediated by a p38 MAPK pathway is an inhibitor of MK2, MK3, MNK1, MNK2, MSK1, MSK2, MSK3, RSK, PP2A, and/or cPLA2.

In some embodiments, those of skill in the art will appreciate that an inhibitor of a proinflammatory immune response mediated by a p38 MAPK pathway (e.g., a p38 MAPK inhibitor) may modulate (e.g., inhibit) activity and/or expression of cyclooxygenase (COX)-1 and/or COX-2. See, e.g., Matsui et al. “Release of prostaglandin E2 and nitric oxide from spinal microglia is dependent on activation of p38 mitogen-activated protein kinase” Anesthesia & Analgesia, 111(2): 554-560 (2010).

In some embodiments, those skilled in the art will appreciate that certain COX inhibitors (e.g., COX-1 and/or COX-2 inhibitors) and/or other anti-inflammatory agents (e.g., non-sterodial anti-inflammatory drugs (NSAIDs) and/or anti-inflammatory analgesics) may act as modulators (e.g., inhibitors) of a p38 MAPK pathway or component(s) thereof (see, e.g., as described in Esposito et al., “Non-steroidal anti-inflammatory drugs in Parkinson's disease” Experimental Neurology 205: 295-312 (2007); Desai et al., “Mechanisms of Phytonutrient Modulation of Cyclooxygenase-2 (COX-2) and Inflammation Related to Cancer” Nutrition and Cancer, 70: 350-375 (2018); Huang et al., “MAPK/ERK signal pathway involved expression of COX-2 and VEGF by IL-1beta induced in human endometriosis stomal cells in vitro” Int J Clin Exp Pathol, 6: 2129-2136 (2013); and Di Mari et al., “HETEs enhance IL-1-mediated COX-2 expression via augmentation of message stability in human colonic myofibroblasts” Am J Physiol-Gastrointest Liver Physiol., 293: 2092-2101 (2007)). Thus, in some embodiments, a COX-2 inhibitor or other anti-inflammatory agent may be (and/or may be used as) a p38 MAPK inhibitor as described herein; alternatively or additionally, in some embodiments, such a COX inhibitor or other anti-inflammatory agent may be utilized in combination with another p38 MAPK inhibitor as described herein.

In some embodiments, a COX inhibitor may be a non-selective COX-1 and/or COX-2 inhibitor. In some embodiments, a COX inhibitor may be a selective COX-1 and/or COX-2 inhibitor.

In some embodiments, certain COX inhibitors that may be useful as p38 MAPK pathway inhibitors (i.e., inhibitors of a p38 MAPK pathway or component(s) thereof) include, but are not limited to, non-steroidal anti-inflammatory drugs (NSAIDs). In some embodiments, NSAIDs can decrease inflammation, e.g., by inhibiting the activity of cyclooexygenase (COX-1 and/or COX-2) enzymes, which are typically involved in production of prostaglandins, which are involved in inflammation.

In some embodiments, a NSAID for use as a p38 MAPK pathway inhibitor is or comprises celecoxib, which is typically known as a selective COX-2 inhibitor. See, e.g., Chen et al., “Celexocib inhibits the lytic activation of Kaposi's sarcoma-associated herpesvirus through down-regulation of RTA expression by inhibiting the activation of p38 MAPK” Viruses 7:2268-2287 (2015); and Fan et al., “Celecoxib attenuates systemic lipopolysaccharide-induced brain inflammation and white matter injury in the neonatal rats” Neuroscience 240: 27-38 (2013).

In some embodiments, a NSAID for use as a p38 MAPK pathway inhibitor is or comprises ketorolac. Ketorolac is known to inhibit prostaglandin synthesis, e.g., by competitive blocking of a COX enzyme. In some embodiments, ketorolac can reduce expression of IL-6. See, e.g., Singh et al., “A prospective study to assess the levels of interleukin-6 following administration of diclofenac, ketorolac and tramadol after surgical removal of lower third molars” J. Maxillofac Oral Surg. 14: 219-225 (2015). Those of skill in the art will also appreciate that ketorolac may act as a non-selective COX inhibitor with known anti-inflammatory properties. However, without wishing to be bound by a particular theory, in some embodiments, ketorolac may be considered to have a higher selectivity for inhibiting COX-1 over COX-2 (See Hersh and Dionne “Nonopioid analgesics” in Pharmacology and Therapeutics for Dentistry (7^{th} edition), Dowd et al., Elsevier Inc. 2017). Ketorolac has been conventionally used for short-term pain management and, therefore, is typically not prescribed for longer than five days. In some embodiments, ketorolac for use in the present disclosure is released from a biomaterial (e.g., as described herein) over a period of at least 5 days or longer, e.g., at least 6 days, at least 7 days, at least 8 days, at least 9 days, at least 10 days, or longer such that immunosuppressive inflammation induced by tumor resection surgery is inhibited or reduced. Ketorolac may be administered as a racemic mixture or as an individual enantiomer, e.g., the S-enantiomer.

Other examples of NSAIDs that are useful in accordance with the present disclosure include, but are not limited to, (i) salicylates (e.g., acetylsalicylic acid, diflunisal, salicylic acid and other salicylates, and/or salsalate); (ii) propionic acid derivatives (e.g., ibuprofen, dexiburofen, naproxen, fenoprofen, ketoprofen, dexketoprofen, flurbiprofen, oxaprozin, and/or loxoprofen; (iii) acetic acid derivatives (e.g., indomethacin, tolmetin, sulindac, etodolac, ketorolac, diclofenac, aceclofenac, and/or nabumetone); (iv) enolic acid (oxicam) derivatives (e.g., piroxicam, meloxicam, tenoxicam, droxicam, lornoxicam, isoxicam, and/or phenylbutazone); (v) anthranilic acid derivatives or fenamates (e.g., mefenamic acid, meclofenamic acid, flufenamic acid, and/or tolfenamic acid); (vi) selective COX-2 inhibitors (e.g., celecoxib, rofecoxib, valdecoxib, parecoxib, lumiracoxib, etoricoxib, and/or firocoxib); (vii) sulfonanilides (e.g., nimesulide); (viii) others (e.g., clonixin, licofelone [e.g., acts by inhibiting lipoxygenase (LOX) and COX], and/or H-harpagide), and combinations thereof.

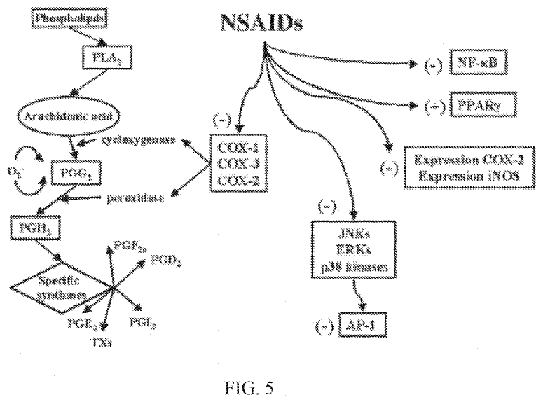

As will be recognized by one of those skilled in the art, in some embodiments, a p38 MAPK inhibitor may modulate (e.g., inhibit) a Wnt-β-catenin pathway or component(s) thereof, e.g., as shown in FIG. 5 and described in Bikkavilli et al., “p38 mitogen-activated protein kinase regulates canonical Wnt-β-catenin signaling by inactivation of GSK3β” Journal of Cell Science, 121: 3598-3607 (2008). Accordingly, in some embodiments, an inhibitor of a proinflammatory immune response mediated by a p38 MAPK pathway may be or comprise a Wnt inhibitor. In some embodiments, an inhibitor of a proinflammatory immune response mediated by a p38 MAPK pathway may be or comprise a GSK3β inhibitor. In some embodiments, an inhibitor of a proinflammatory immune response mediated by a p38 MAPK pathway may be or comprise a β-catenin inhibitor.

Those skilled in the art will also appreciate that certain Wnt/β-catenin pathway inhibitors may act as modulators (e.g., inhibitors) of a p38 MAPK pathway or component(s) thereof (see, e.g., as described in Andre et al., “Wnt5a and Wnt11 regulate mammalian anterior-posterior axis elongation” Development 142: 1516-1527 (2015); and Ma et al., “Crosstalk between Wnt/β-catenin and NF-κB signaling pathway during inflammation” Front Immunol. 7: 378 (2016)). Thus, in some embodiments, a Wnt/β-catenin pathway inhibitor may be (and/or may be used as) a p38 MAPK inhibitor as described herein; alternatively or additionally, in some embodiments such a Wnt/β-catenin pathway inhibitor may be utilized in combination with another p38 MAPK inhibitor as described herein.

### Activator of Innate Immune Response

The drug delivery compositions and devices may comprise an activator of innate immune response. The drug delivery compositions and devices may comprise more than one activator of innate immune response. The major functions of the innate immune response include recruiting immune cells to sites of infection through the production of chemical factors, including specialized chemical mediators (e.g., cytokines); activation of the complement cascade to identify bacteria, activate cells, and promote clearance of antibody complexes or dead cells; identification and removal of foreign substances present in organs, tissues, blood, and lymph by specialized white blood cells; activation of the adaptive immune system through a process known as antigen presentation; and acting as a physical and chemical barrier to infectious agents (e.g., epithelial surfaces, gastrointestinal tract). Typically, leukocytes are the white blood cells that carry out the actions of the innate immune system. These cells include natural killer cells, mast cells, eosinophils, basophils, macrophages, neutrophils, and dendritic cells. These cells function within the immune system by identifying and eliminating pathogens that might cause infection.

In certain embodiments, the the activator of innate immune response is a ligand of a pattern recognition receptor (PRR).

In certain embodiments, the activator of innate immune response is an agonist of a pattern recognition receptor (PRR).

In certain embodiments, the activator of innate immune response is an inducer of type I interferon. In certain embodiments, the activator of innate immune response is a recombinant interferon.

In certain embodiments, the activator of innate immune response is an effective inducer of activation and/or proliferation of NK cells. In certain embodiments, “effective inducer” refers to an activator of innate immune response that directly induces activation and/or proliferation of NK cells.

In certain embodiments, the activator of innate immune response is an effective inducer of activation and/or maturation of dendritic cells. In certain embodiments, “effective inducer” refers to an activator of innate immune response that directly induces activation and/or maturation of dendritic cells.

In certain embodiments, the activator of innate immune response is an effective inducer of type I interferon by dendritic cells. In certain embodiments, “effective inducer” refers to an activator of innate immune response that directly induces type I interferon by dendritic cells.

In certain embodiments, the activator of innate immune response is a small molecule or a biologic. In certain embodiments, the activator of innate immune response is a small molecule. In certain embodiments, the activator of innate immune response is a biologic.

In certain embodiments, the activator of innate immune response is a stimulator of interferon genes (STING) agonist, a cytosolic DNA sensor (CDS) agonist, a Toll-like receptor (TLR) agonist, a C-type lectin receptor (CLR) agonist, a NOD-like receptor (NLR) agonist, a RIG-I-like receptor (RLR) agonist, or an inflammasome inducer.

In certain embodiments, the activator of innate immune response is a stimulator of interferon genes (STING) agonist, a Toll-like receptor (TLR) agonist, or a NOD-like receptor (NLR) agonist. In certain embodiments, the activator of innate immune response is a stimulator of interferon genes (STING) agonist or a Toll-like receptor (TLR) agonist. In certain embodiments, the activator of innate immune response is a stimulator of interferon genes (STING) agonist, a TLR7 agonist, or a TLR8 agonist.

In certain embodiments, the activator of innate immune response is 3′3′-cGAMP, 2′3′-cGAMP, 2′3′-cGAM(PS)2 (Rp/Rp), 2′3′-cGAM(PS)2 (Rp/Sp), 2′2′-cGAMP, c-di-AMP, 2′3′-c-di-AMP, 2′3′-c-di-AMP(PS)2 (Rp/Rp), 2′3′-c-di-AMP(PS)2 (Rp/Sp), c-di-GMP, c-di-IMP, HSV-60, ISD, VACV-70, poly(dA:dT), poly(dG:dC), heat-killed bacteria, lipoglycans, lipopolysaccharides (LPS), lipoteichoic acids, peptidoglycans (PGNs), synthetic lipoproteins, poly(A:U), poly(I:C), Monophosphoryl Lipid A (MPLA), GSK1795091, G100, SD-101, MGN1703, CMP-001, flagellin (FLA), polyU, poly(dT), gardiquimod, imiquimod (R837), base analogs, adenine analogs, guanosin analogs, purine derivatives, benoazepine analogs, imidazoquinolines, thiazoquinolines, loxoribine, resiquimod (R848), dactolisib, sumanirole, N1-glycinyl[44(6-amino-2-(butylamino)-8-hydroxy-9H-purin-9-yl)methyl) benzoyl] spermine (CL307), CL264, CL097, CL075, CL347, CL401, CL413, CL419, CL531, CL553, CL572, MEDI9197, MEDI5083, hypoxanthine, TL8-506, PF-4878691, isatoribine, SM-324405, SM-324406, AZ12441970, AZ12443988, CpG oligonucleotides, bacterial DNA, beta glucans, beta glucans from fungal and bacterial cell walls, γ-D-Glu-mDAP (iE-DAP), iE-DAP derivatives, muramyl dipeptide (MDP), MDP derivatives, 5′ triphosphate double stranded RNA, poly(dA:dT), ATP, chitosan, aluminum potassium sulfate, calcium pyrophosphate dehydrate, silica dioxide, MurNAc-L-Ala-γ-D-Glu-mDAP (M-TriDAP), a xanthenone analog (e.g., DMXAA; vadimezan), a TREX1 inhibitor, a cyclic dinucleotide, LHC165, GSK-2245035, RG7854, GS-9620, GS-9688, EMD1201081, PF-3512676, BO-112, RGT-100, MK-1454, SB-11285, NKTR-262, CDX-301, 2′3′-c-di-GMP, cAIMP, cAIM(PS)2 (Rp/Sp), derivatives thereof, or pharmaceutically acceptable salts thereof.

In certain embodiments, the activator of innate immune response is a fluorinated derivative of any of the above activators. In certain embodiments, the activator of innate immune response is difluoroinated cAIMP (c-(2′FdAMP-2′FdIMP)). In certain embodiments, the activator of innate immune response is difluoroinated cAIM(PS)2 (Rp/Sp). In certain embodiments, the activator of innate immune response is an O-methylated derivative of any of the above activators.

In certain embodiments, the activator of innate immune response is 3′3′-cGAMP, 2′3′-cGAMP, 2′3′-cGAM(PS)2 (Rp,Rp), 2′3′-cGAM(PS)2 (Rp,Sp), 2′2′-cGAMP, c-di-AMP, 2′3′-c-di-AMP, 2′3′-c-di-AM(PS)2 (Rp,Rp), 2′3′-c-di-AM(PS)2 (Rp,Sp), c-di-GMP, 2′3′-c-di-GMP, 2′3′-c-di-GM(PS)2 (Rp,Rp), 2′3′-c-di-GM(PS)2 (Rp,Sp), c-di-IMP, resiquimod, CpG oligonucleotides, polyinosinic:polycytidylic acid, LHC165, GSK-2245035, RG7854, GS-9620, GS-9688, EMD1201081, PF-3512676, BO-112, RGT-100, MK-1454, SB-11285, NKTR-262, CDX-301, 2′3′-c-di-GMP, cAIMP, cAIM(PS)2 (Rp/Sp), or pharmaceutically acceptable salts thereof.

In certain embodiments, the activator of innate immune response is a fluorinated derivative of 3′3′-cGAMP, 2′3′-cGAMP, 2′3′-cGAM(PS)2 (Rp,Rp), 2′3′-cGAM(PS)2 (Rp,Sp), 2′2′-cGAMP, c-di-AMP, 2′3′-c-di-AMP, 2′3′-c-di-AM(PS)2 (Rp,Rp), 2′3′-c-di-AM(PS)2 (Rp,Sp), c-di-GMP, 2′3′-c-di-GMP, 2′3′-c-di-GM(PS)2 (Rp,Rp), 2′3′-c-di-GM(PS)2 (Rp,Sp), c-di-IMP, 2′3′-c-di-GMP, cAIMP, cAIM(PS)2 (Rp/Sp), or pharmaceutically acceptable salts thereof.

In certain embodiments, the activator of innate immune response is an O-methylated derivative of 3′3′-cGAMP, 2′3′-cGAMP, 2′3′-cGAM(PS)2 (Rp,Rp), 2′3′-cGAM(PS)2 (Rp,Sp), 2′2′-cGAMP, c-di-AMP, 2′3′-c-di-AMP, 2′3′-c-di-AM(PS)2 (Rp,Rp), 2′3′-c-di-AM(PS)2 (Rp,Sp), c-di-GMP, 2′3′-c-di-GMP, 2′3′-c-di-GM(PS)2 (Rp,Rp), 2′3′-c-di-GM(PS)2 (Rp,Sp), c-di-IMP, or pharmaceutically acceptable salts thereof.

In certain embodiments, the activator of innate immune response is 2′3′-cGAMP, 2′3′-c-di-AM(PS)2 (Rp,Rp), MurNAc-L-Ala-γ-D-Glu-mDAP (M-TriDAP), c-di-GMP, or resiquimod. In certain embodiments, the activator of innate immune response is 2′3′-cGAMP, 2′3′-c-di-AM(PS)2 (Rp,Rp), MurNAc-L-Ala-γ-D-Glu-mDAP (M-TriDAP), or resiquimod. In certain embodiments, the activator of innate immune response is 2′3′-cGAMP, 2′3′-c-di-AM(PS)2 (Rp,Rp), or resiquimod. In certain embodiments, the activator of innate immune response is 2′3′-c-di-AM(PS)2 (Rp,Rp) or resiquimod. In certain embodiments, the activator of innate immune response is cAIMP or its fluorinated derivative. In certain embodiments, the activator of innate immune response is difluorinated cAIMP.

In certain embodiments, the activator of innate immune response is 2′3′-cGAMP, or a pharmaceutically acceptable salt thereof. In particular, 2′3′-cGAMP (cyclic [G(2′,5′)pA(3′,5′)p]) has been described to function as an endogenous second messenger, inducing STING-dependent type I interferon response. 2′3′-cGAMP has also been shown to be an effective adjuvant that boosts the production of antigen-specific antibodies and T cell responses in mice. 2′3′-cGAMP exercises antiviral functions in the cell where it is produced but can also cross cell membranes by passive diffusion to exert effects on neighboring cells.

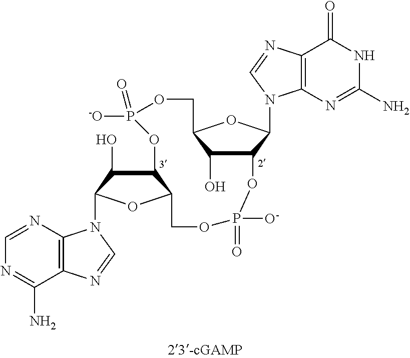

In certain embodiments, the activator of innate immune response is 2′3 ‘-c-di-AM(PS)2 (Rp,Rp), or a pharmaceutically acceptable salt thereof. 2′3’-c-di-AM(PS)2 (Rp,Rp) is the Rp,Rp-isomer of the 2′3′ bisphosphorothioate analog of 3′3′-cyclic adenosine monophosphate (c-di-AMP). It is also a STING agonist.

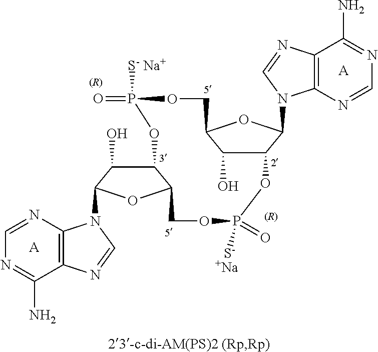

In certain embodiments, the activator of innate immune response is cAIMP, its difluorinated derivative, its difluorinated bisphosphorothiate derivate (cAIM(PS)2 (Rp/Sp)), or a pharmaceutically acceptable salt thereof cAIMP and its derivatives are also STING agonists.

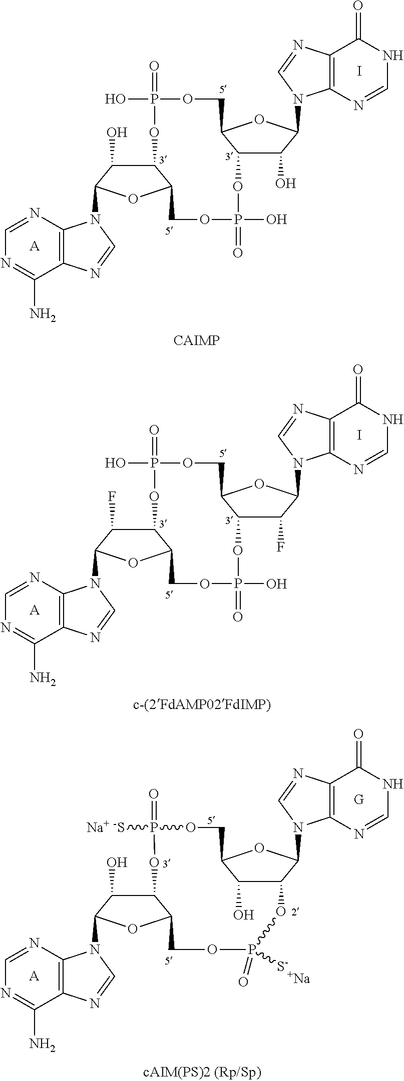

In certain embodiments, the activator of innate immune response is a STING agonist, wherein the STING agonist is a cyclic dinucleotide. In certain embodiments, the cyclic dinucleotide is any cyclic dinucleotide disclosed in U.S. patent application U.S. Ser. No. 15/234,182, filed Aug. 11, 2016, the entire contents of which are incorporated herein by reference. In certain embodiments, the cyclic dinucleotide is any cyclic dinucleotide disclosed in U.S. patent application U.S. Ser. No. 14/362,441, filed Dec. 16, 2014, the entire contents of which are incorporated herein by reference.

In certain embodiments, the activator of innate immune response is MK-1454.

In certain embodiments, the activator of innate immune response is a cytosolic DNA sensor (CDS) agonist. In certain embodiments, the CDS agonist is a cyclic GMP-AMP synthase (cGAS) agonist.

In certain embodiments, the activator of innate immune response is any STING agonist or cGAS agonist disclosed in U.S. patent application U.S. Ser. No. 14/653,586, filed Dec. 16, 2013, the entire contents of which are incorporated herein by reference. In certain embodiments, the activator of innate immune response is any STING agonist or cGAS agonist disclosed in U.S. patent application U.S. Ser. No. 14/268,967, filed May 2, 2014, the entire contents of which are incorporated herein by reference. In certain embodiments, the activator of innate immune response is any STING agonist or cGAS agonist disclosed in U.S. patent application U.S. Ser. No. 14/787,611, filed Apr. 29, 2014, the entire contents of which are incorporated herein by reference. In certain embodiments, the activator of innate immune response is any STING agonist or cGAS agonist disclosed in U.S. patent application U.S. Ser. No. 14/908,019, filed Jul. 31, 2014, the entire contents of which are incorporated herein by reference.

In certain embodiments, the activator of innate immune response is any STING agonist disclosed in U.S. patent application U.S. Ser. No. 13/057,662, filed Jun. 14, 2011, the entire contents of which are incorporated herein by reference. In certain embodiments, the activator of innate immune response is any STING agonist disclosed in U.S. patent application U.S. Ser. No. 14/106,687, filed Dec. 13, 2013, the entire contents of which are incorporated herein by reference. In certain embodiments, the activator of innate immune response is any STING agonist disclosed in U.S. patent application U.S. Ser. No. 15/035,432, filed May 19, 2016, the entire contents of which are incorporated herein by reference. In certain embodiments, the activator of innate immune response is any STING agonist disclosed in International Patent Application PCT/US2017/013049, filed Jan. 11, 2017, the entire contents of which are incorporated herein by reference. In certain embodiments, the activator of innate immune response is any STING agonist disclosed in International Patent Application PCT/US2017/013066, filed Jan. 11, 2017, the entire contents of which are incorporated herein by reference. In certain embodiments, the activator of innate immune response is any STING agonist disclosed in International Patent Application PCT/US2014/038525, filed May 18, 2014, the entire contents of which are incorporated herein by reference. In certain embodiments, the activator of innate immune response is any STING agonist disclosed in U.S. patent application U.S. Ser. No. 13/912,960, filed Jun. 7, 2013, the entire contents of which are incorporated herein by reference. In certain embodiments, the activator of innate immune response is any STING agonist disclosed in International Patent Application PCT/IB2016/057265, filed Jan. 12, 2016, the entire contents of which are incorporated herein by reference.

In certain embodiments, the activator of innate immune response is MurNAc-L-Ala-γ-D-Glu-mDAP (M-TriDAP), or a pharmaceutically acceptable salt thereof. M-TriDAP is a peptidoglycan (PGN) degradation product found mostly in Gram-negative bacteria. M-TriDAP is recognized by the intracellular sensor NOD1 (CARD4) and to a lesser extent NOD2 (CARD15). Recognition of M-TriDAP by NOD1/NOD2 induces a signaling cascade involving the serine/threonine RIP2 (RICK, CARDIAK) kinase, which interacts with IKK leading to the activation of NF-κB and the production of inflammatory cytokines such as TNF-α and IL-6. M-TriDAP induces the activation of NF-κB at similar levels to Tri-DAP.

In certain embodiments, the activator of innate immune response is a TLR7 agonist. In certain embodiments, the activator of innate immune response is a TLR8 agonist. In certain embodiments, the activator of innate immune response is a TLR7 agonist and a TLR8 agonist.

In certain embodiments, the activator of innate immune response is an immune response modifier (IRM).

In certain embodiments, the activator of innate immune response is any IRM disclosed in U.S. patent application U.S. Ser. No. 08/620,779, filed Mar. 22, 1996, the entire contents of which are incorporated herein by reference. In certain embodiments, the activator of innate immune response is any IRM disclosed in U.S. patent application U.S. Ser. No. 08/957,192, filed Oct. 24, 1997, the entire contents of which are incorporated herein by reference. In certain embodiments, the activator of innate immune response is any IRM disclosed in U.S. patent application U.S. Ser. No. 09/528,620, filed Mar. 20, 2000, the entire contents of which are incorporated herein by reference. In certain embodiments, the activator of innate immune response is any IRM disclosed in U.S. patent application U.S. Ser. No. 06/798,385, filed Nov. 15, 1985, the entire contents of which are incorporated herein by reference. In certain embodiments, the activator of innate immune response is any IRM disclosed in U.S. patent application U.S. Ser. No. 08/303,216, filed Sep. 8, 1994, the entire contents of which are incorporated herein by reference. In certain embodiments, the activator of innate immune response is any IRM disclosed in U.S. patent application U.S. Ser. No. 09/210,114, filed Dec. 11, 1998, the entire contents of which are incorporated herein by reference. In certain embodiments, the activator of innate immune response is any IRM disclosed in U.S. patent application U.S. Ser. No. 09/361,544, filed Jul. 27, 1999, the entire contents of which are incorporated herein by reference. In certain embodiments, the activator of innate immune response is any IRM disclosed in International Patent Application, PCT/US2004/032480, filed Oct. 1, 2004, the entire contents of which are incorporated herein by reference.

In certain embodiments, the activator of innate immune response is CL307 (N1-glycinyl[4-((6-amino-2-(butylamino)-8-hydroxy-9H-purin-9-yl)methyl) benzoyl] spermine), or a pharmaceutically acceptable salt thereof. CL307 is a very potent TLR7 agonist. Titration experiments have showed that CL307 induces robust NF-κB activation even at concentrations as low as 20 nM (10 ng/ml).

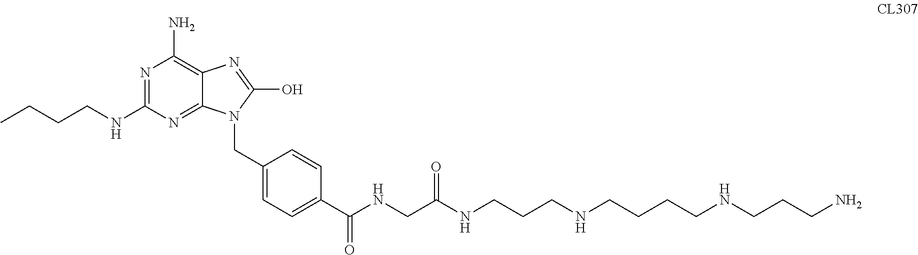 [SMILES: CCCCNc1nc(N)c2nc(O)n(Cc3ccc(C(=O)NCC(=O)NCCCNCCCCNCCCN)cc3)c2n1]

In certain embodiments, the activator of innate immune response is CL264, or a pharmaceutically acceptable salt thereof. CL264 induces the activation of NF-κB and the secretion of IFN-α in TLR7-expressing cells. CL264 is a TLR7-specific ligand, it does not stimulate TLR8 even at high concentrations (>10 μg/ml). In TLR7-transfected HEK293 cells, CL264 triggers NF-κB activation at a concentration of 0.1 μM which is 5-10 times less than imiquimod.

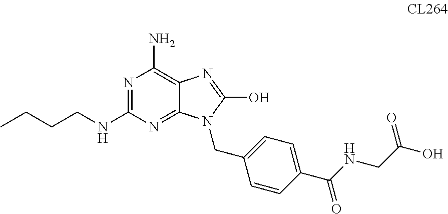 [SMILES: CCCCNc1nc(N)c2nc(O)n(Cc3ccc(C(=O)NCC(=O)O)cc3)c2n1]

In certain embodiments, the activator of innate immune response is loxoribine, or a pharmaceutically acceptable salt thereof. Loxoribine is a guanosine analog derivatized at positions N⁷ and C⁸. This nucleoside is a very powerful stimulator of the immune system. Loxoribine activates the innate immune system through TLR7 and this activation requires endosomal maturation. Loxoribine recognition is restricted to TLR7.

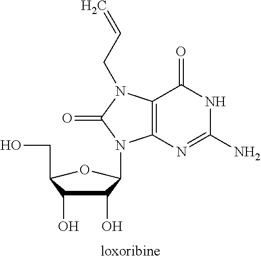

In certain embodiments, the activator of innate immune response is hypoxanthine, or a pharmaceutically acceptable salt thereof. Hypoxanthine is a naturally occurring purine derivative.

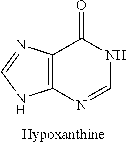

In certain embodiments, the activator of innate immune response is TL8-506, or a pharmaceutically acceptable salt thereof. TL8-506 is a benzoazepine compound, an analog of the Toll-like receptor 8 (TLR8) agonist VTX-2337. TL8-506 activates TLR8 more potently than R848 and CL075. TL8-506 is ˜50× and ˜25× more potent in inducing NF-κB activation in TLR8-transfected HEK293 cells than R848 and CL075, respectively. TL8-506 is a selective agonist of TLR8.

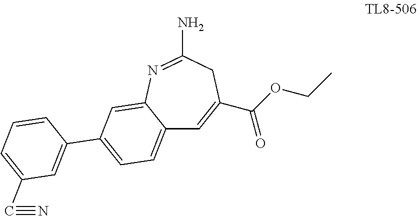 [SMILES: CCOC(=O)C1=Cc2ccc(-c3cccc(C#N)c3)cc2N=C(N)C1]

In certain embodiments, the activator of innate immune response is PF-4878691, isatoribine, SM-324405, SM-324406, AZ12441970, AZ12443988, GSK-2245035, RG7854, GS-9620, LHC165, NKTR-262, GS-9688, VTX-2337, or pharmaceutically acceptable salts thereof. PF-4878691, isatoribine, SM-324405, SM-324406, AZ12441970, AZ12443988, GSK-2245035, RG7854, and GS-9620 are TLR7 agonists. LHC165 and NKTR-262 are agonists of both TLR7 and TLR8 agonists. GS-9688 and VTX-2337 are TLR8 agonists.

In certain embodiments, the activator of innate immune response is an imidazoquinoline derivative, including dactolisib, imiquimod, gardiquimod, resiquimod, sumanirole, and pharmaceutically acceptable salts thereof.

In certain embodiments, the activator of innate immune response is CL097, or a pharmaceutically acceptable salt thereof. CL097 is a highly water-soluble derivative resiquimod (≥20 mg/ml). CL097 is a TLR7 and TLR8 ligand. It induces the activation of NF-κB at 0.4 μM (0.1 μg/ml) in TLR7-transfected HEK293 cells and at 4 μM (1 μg/ml) in TLR8-transfected HEK293 cells.

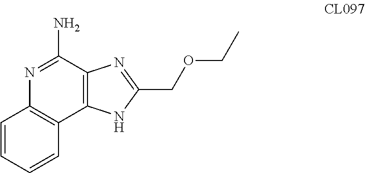 [SMILES: CCOCc1nc2c(N)nc3ccccc3c2[nH]1]

In certain embodiments, the activator of innate immune response is CL075, or a pharmaceutically acceptable salt thereof. CL075 (3M002) is a thiazoloquinolone derivative that stimulates TLR8 in human peripheral blood mononuclear cells. It activates NF-κB and triggers preferentially the production of TNF-α and IL-12. CL075 also induces the secretion of IFN-α through TLR7, but to a lesser extent. It induces the activation of NF-κB at 0.4 μM (0.1 μg/ml) in TLR8-transfected HEK293 cells, and ˜10 times more CL075 is required to activate NF-κB in TLR7-transfected HEK293 cells.

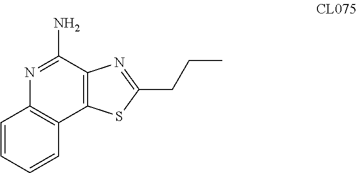 [SMILES: CCCc1nc2c(N)nc3ccccc3c2s1]

In certain embodiments, the activator of innate immune response is MEDI9197, or a pharmaceutically acceptable salt thereof. MEDI9197 (3M052) is an injectable TLR7 and TLR8 agonist. It is an imidazoquinoline immune response modifier (IRM) bearing a C18 lipid moiety and designed for slow dissemination from the site of application.

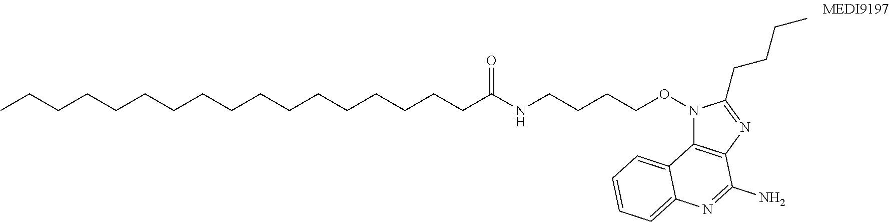 [SMILES: CCCCCCCCCCCCCCCCCC(=O)NCCCCOn1c(CCCC)nc2c(N)nc3ccccc3c21]

In certain embodiments, the activator of innate immune response is resiquimod (R848), or a pharmaceutically acceptable salt thereof. In particular, resiquimod is an agent that acts as an immune response modifier and has antiviral and antitumor activity. It is used as a topical gel in the treatment of skin lesions such as those caused by the herpes simplex virus and cutaneous T cell lymphoma. It is also used as an adjuvant to increase the effectiveness of vaccines. It has several mechanisms of action, being both an agonist for toll-like receptor 7 (TLR7) and 8 (TLR8), and an upregulator of the opioid growth factor receptor.

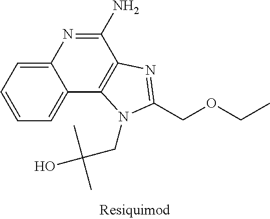 [SMILES: CCOCc1nc2c(N)nc3ccccc3c2n1CC(C)(C)O]

In certain embodiments, the activator of innate immune response is a TLR7-selective antedrug. In certain embodiments, the activator of innate immune response is SM-324405, AZ12441970, or pharmaceutically acceptable salts thereof.

In certain embodiments, the activator of innate immune response is GS-9620. In certain embodiments, the activator of innate immune response is PF-4878691. In certain embodiments, the activator of innate immune response is NKTR-262. In certain embodiments, the activator of innate immune response is LHC165.

In certain embodiments, the activator of innate immune response is an inflammasome inducer. Inflammasomes are multimeric protein complexes that are crucial for host defense to infection and endogenous danger signals. They promote the secretion of the proinflammatory cytokines interleukin (IL)-1β and IL-18 and cause a rapid and proinflammatory form of cell death called pyroptosis.

In certain embodiments, the activator of innate immune response is an inducer of NLRP3, AIM2, NLRC4, or NLRP1 inflammasomes.

In certain embodiments, the activator of innate immune response is

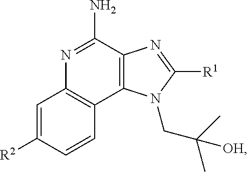

or a pharmaceutically acceptable salt thereof, wherein: R¹ is H, and R² is H; R¹ is a butyl group and R² is H; R¹ is H and R² is —CO₂CH₃; or R¹ is a butyl group and R² is —CO₂CH₃.

In certain embodiments, the activator of innate immune response is an imadazoquinoline; an imidazonaphthyridine; a pyrazolopyridine; an aryl-substituted imidazoquinoline; a compound having a 1-alkoxy 1H-imidazo ring system; an oxazolo [4,5-c]-quinolin-4-amine; a thiazolo [4,5-c]-quinolin-4-amine; a selenazolo [4,5-c]-quinolin-4-amine; an imidazonaphthyridine; an imidazoquinolinamine; a 1-substituted, 2-substituted 1H-imidazo[4,5-C]quinolin-4-amine; a fused cycloalkylimidazopyridine; a 1H-imidazo[4,5-c]quinolin-4-amine; a 1-substituted 1H-imidazo-[4,5-c]quinolin-4-amine; an imidazo-[4,5-C]quinolin-4-amine; a 2-ethyl 1H-imidazo[4,5-ciquinolin-4-amine; an olfenic 1H-imidazo[4,5-c]quinolin-4-amine; a 6,7-dihydro-8-(imidazol-1-yl)-5-methyl-1-oxo-1H,5H-benzo[ij]quinolizine-2-carboxylic acid; a pyridoquinoxaline-6-carboxylic acid; a 6,7-dihydro-8-(imidazol-1-yl)-5-methyl-1-oxo-1H,5H-benzo [ij]quinolizine-2-carboxylic acid; a substituted naphtho[ij]quinolizine; a substituted pyridoquinoxaline-6-carboxylic acid; a 7-hydroxy-benzo[ij]quinolizine-2-carboxylic acid derivative; a substituted benzo[ij]quinolizine-2-carboxylic acid; a 7-hydroxy-benzo[ij]quinolizine-2-carboxylic acid; a substituted pyrido[1,2,3,-de]-1,4-benzoxazine; a N-methylene malonate of tetrahydroquinoline, or pharmaceutically acceptable salts thereof.

In certain embodiments, the activator of innate immune response is any NLRP3 agonist disclosed in U.S. patent application U.S. Ser. No. 15/253,215, filed Aug. 31, 2016, the entire contents of which are incorporated herein by reference.

In certain embodiments, the activator of innate immune response is a RORγ agonist. A RORγ agonist is an agent that promotes RORγ activity, such as by binding to and activating RORγ or by increasing expression of RORγ in a patient or population of cells. The RORγ agonist may be, for example, a small organic molecule, polypeptide, or nucleic acid. Various RORγ agonists are reported in the literature, such as in U.S. patent application U.S. Ser. No. 14/398,774; Zhang et al. in Mol. Pharmacol. (2012) vol. 82, pages 583-590; and Wang et al. in ACS Chem. Biol. (2010), vol. 5, pages 1029-1034; each of which is hereby incorporated by reference.

In certain embodiments, the activator of innate immune response is a RORγ agonist, such as

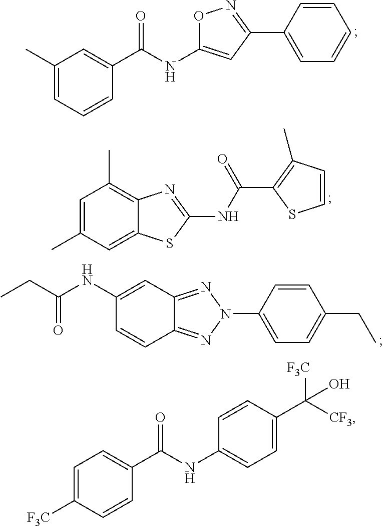

and pharmaceutically acceptable salts thereof.

In certain embodiments, the activator of innate immune response is a generic or specific compound described in U.S. patent application U.S. Ser. No. 14/398,774, such as a compound of Formula (I):

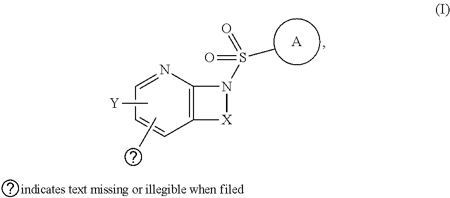

or a pharmaceutically acceptable salt thereof; wherein:

A is aryl, aralkyl, heteroaryl, cycloalkyl, or heterocycloalkyl; each of which is optionally substituted with 1, 2, or 3 substituents independently selected from the group consisting of halogen, hydroxyl, C₁₋₆ alkyl, C₁₋₆ haloalkyl, C₁₋₆ hydroxyalkyl, C₁₋₆ alkoxy, C₁₋₆ haloalkoxy, — [symbol] N(R⁴)(R⁵), —CO₂R⁶, —C(O)R⁶, —CN, —C₁₋₄alkylene-C₁₋₄alkoxy, —C₁₋₄alkylene-N(R⁴)(R⁵), —C₁₋₄alkylene-CO₂R⁶, —O—C₁₋₆alkylene-N(R⁴)(R⁵), —N(R⁴)C(O)—C₁₋₆alkylene-N(R⁴)(R⁵), —S(O)ₚC₁₋₆alkyl, —SO₂N(R⁴)(R⁵), —N(R⁴)SO₂ (C₁₋₆alkyl), —C(O)N(R⁴)(R⁵), and N(R⁴)C(O)N(R⁴)(R⁵);

X is —O—[C(R⁶)(R⁷)]—[C(R⁶)₂]ₘ-Ψ, —O—C(R⁶)₂—C(R⁶)(R⁷)—C(R⁶)₂-Ψ, —O—C(R⁶)₂—C(R⁶)(R⁷)— Ψ, —C(R⁶)₂—[C(R⁶)(R⁷)]—[C(R⁶)₂]m-Ψ, —C(O)—[C(R⁶)(R⁷)]—[C(R⁶)₂]m-Ψ, —C(R⁶)₂—N(R⁸)—[C(R⁶)(R⁷)]—[C(R⁶)₂]m-Ψ, —C(R⁶)═N-Ψ, C(R⁶)₂C(R⁶)═N-Ψ, —N═C(R⁶)— Ψ, or —N═C(R⁶)C(R⁶)₂-Ψ; wherein Ψ is a bond to the sulfonamide ring nitrogen atom in Formula I;

Y is —N(R²)(R³) or —O-aralkyl, wherein said aralkyl is optionally substituted with 1, 2, or 3 substituents independently selected from the group consisting of halogen, hydroxyl, C₁₋₆alkoxy, C₁₋₆ haloalkoxy, C₁₋₆ alkyl, C₁₋₆ haloalkyl, —N(R⁴)(R⁵), —CN, —CO₂—C₁₋₆ alkyl, —C(O)—C₁₋₆ alkyl, —C(O)N(R⁴)(R⁵), —S(O)ₚC₁₋₆ alkyl, —SO₂N(R⁴)(R⁵), and —N(R⁴)SO₂(C₁₋₆alkyl);

R¹ represents independently for each occurrence hydrogen, halogen, or C₁₋₆ alkyl;

R² is —C(O)-aryl, —C(O)-aralkyl, —C(O)[C(R⁶)₂]ₘ-cycloalkyl, —C(O)[C(R⁶)₂]ₘ-heterocyclyl, —C(O)—C₁₋₆alkyl, —C(O)—C₁₋₆alkylene-C₁₋₆alkoxyl, —C(O)—C₁₋₆alkylene-cycloalkyl, or —C(O)—C₁₋₆ alkylene-heterocycloalkyl; each of which is optionally substituted with 1, 2, or 3 substituents independently selected from the group consisting of halogen, hydroxyl, C₁₋₆ alkoxy, C₁₋₆ haloalkoxy, C₁₋₆ alkyl, C₁₋₆ haloalkyl, —N(R⁴)(R⁵), —CN, —CO₂—C₁₋₆alkyl, —C(O)—C₁₋₆ alkyl, —C(O)N(R⁴)(R⁵), —S(O)ₚC₁₋₆ alkyl, —SO₂N(R⁴)(R⁵), and N(R⁴)SO₂(C₁₋₆ alkyl);

R³ is hydrogen or C₁₋₆ alkyl;

R⁴ and R⁵ each represent independently for each occurrence hydrogen or C₁₋₆ alkyl; or R⁴ and R⁵ taken together with the nitrogen atom to which they are attached form a 3-7 membered heterocyclic ring;

R⁶ represents independently for each occurrence hydrogen or C₁₋₆ alkyl;

R⁷ is hydrogen, hydroxyl, C₁₋₆ hydroxyalkyl, C₁₋₆ alkyl, C₁₋₆ haloalkyl, —CO₂R⁶, C₁₋₆alkylene-CO₂R⁶, C₁₋₄ hydroxyalkylene-CO₂R⁶, —N(R⁴)(R⁵), C₁₋₆ alkylene-N(R⁴)(R⁵), C₁₋₆hydroxyalkylene-N(R⁴)(R⁵), —N(R⁴)C(O)R⁹, C₁₋₆ alkylene-N(R⁴)C(O)R⁹, C₁₋₆ alkylene-C(O)N(R⁴)(R⁵), —N(R⁴)CO₂—C₁₋₆ alkyl, or C₁₋₆ alkylene-N(R⁴)(C(O)N(R⁴)(R⁵); or R⁷ is heterocycloalkyl or C₁₋₄ alkylene-heterocycloalkyl, wherein the heterocycloalkyl is optionally substituted with 1, 2, or 3 substituents independently selected from the group consisting of oxo, halogen, hydroxyl, C₁₋₆alkyl, C₁₋₆ haloalkyl, C₁₋₆ hydroxyalkyl, C₁₋₆alkoxy, and C₁₋₆ haloalkoxy;

R⁸ is hydrogen, C₁₋₆ alkyl, or —C(O)—C₁₋₆ alkyl;

R⁹ is hydrogen, C₁₋₆ alkyl, C₁₋₆ hydroxyalkyl, C₁₋₆ alkylene-N(R⁴)(R⁵), or C₁₋₆ alkylene-N(R⁴)C(O)—C₁₋₆ alkyl;

n is 1 or 2; and

m and p each represent independently for each occurrence 0, 1, or 2.

In certain embodiments, the activator of innate immune response is any RORγ agonist disclosed in U.S. patent application U.S. Ser. No. 14/398,774, filed Nov. 4, 2014, the entire contents of which are incorporated herein by reference. In certain embodiments, the activator of innate immune response is any RORγ agonist disclosed in U.S. patent application U.S. Ser. No. 15/120,798, filed Aug. 23, 2016, the entire contents of which are incorporated herein by reference.

In certain embodiments, the activator of innate immune response is a RIG-I-like receptor (RLR) agonist. In certain embodiments, the activator of innate immune response is RGT-100.

### Cytokine

The drug delivery compositions and devices may comprise a cytokine. Cytokines are a broad category of small proteins (˜5-20 kDa) that are important in cell signaling. Their release has an effect on the behavior of cells around them. Cytokines are involved in autocrine signalling, paracrine signaling, and endocrine signaling as immunomodulating agents. Cytokines include chemokines, interferons, interleukins, lymphokines, and tumour necrosis factors. Cytokines are produced by a broad range of cells, including immune cells, such as macrophages, B lymphocytes, T lymphocytes, and mast cells, as well as endothelial cells, fibroblasts, and various stromal cells. They act through receptors and play an important role in the immune system. Cytokines modulate the balance between humoral and cell-based immune responses, and they regulate the maturation, growth, and responsiveness of particular cell populations. Some cytokines enhance or inhibit the action of other cytokines in complex ways. Cytokines are important in host responses to infection, immune responses, inflammation, trauma, sepsis, cancer, and reproduction.

Furthermore, it is currently known in the art that the method of delivery, dosing and scheduling, and toxicity-related issues must be addressed to enable the immune-stimulating function of many cytokines and chemokines to be fully exploited.

In certain embodiments, the cytokine is IL-1, IL-1α, IL-1β, IL-2, an IL-2 superkine, IL-6, IL-7, IL-9, AM0010, IL-12, IL-15, an IL-15 superagonist, ALT-803, NIZ985, IL-16, IL-18, IL-21, an IL-21 superagonist, denenicokin, an IL-21 superagonist antibody, IFN-α, IFN-β, IFN-γ, TNF-α, GM-CSF, a cytokine fusion, RG7461, RG7813, M9241, NKTR-214, NKTR-255, BMS-982470, BG-00001, Flt3L, or CDX-301.

In certain embodiments, the cytokine is ALT-803, NIZ985, denenicokin, RG7461, RG7813, M9241, IFN-α, IFN-β, or IFN-γ.

In certain embodiments, the cytokine is an IL-15 superagonist or IL-21. In certain embodiments, the cytokine is an IL-15 superagonist.

In certain embodiments, the cytokine is an IL-15 superagonist, IL-21, IFN-α, IFN-β, IFN-γ, CCL4, CCL5, CXCL9, or CXCL10. In certain embodiments, the cytokine is an IL-15 superagonist, IFN-α, IFN-β, or IFN-γ. In certain embodiments, the cytokine is an IL-15 superagonist or IFN-α.

IL-15 (Interleukin 15) is a cytokine with structural similarity to IL-2 and is secreted by mononuclear phagocytes following infection by virus(es). IL-15 induces cell proliferation of natural killer cells, cells whose principal role is to kill virally infected cells. The combination of IL-15 with soluble IL-15Rα generates a complex termed IL-15 superagonist (IL-15sa) that possesses greater biological activity than IL-15 alone. IL-15sa is an antitumor and antiviral agent because of its ability to selectively expand NK and memory CD8+T (mCD8+T) lymphocytes.

In certain embodiments, the cytokine is an IL-15 superagonist known as ALT-803, an IL-15 superagonist. ALT-803 is thought to induce memory CD8+ T cells to proliferate, upregulate receptors involved in innate immunity, secrete interferon-y, and acquire the ability to kill malignant cells in the absence of antigenic stimulation. Thus, ALT-803 can promote the expansion and activation of memory CD8+ T cells while converting them into innate immune effector cells that exhibit robust antineoplastic activity. ALT-803 is a fusion protein of an IL-15 mutant and the IL-15Rα/Fc complex that has recently entered clinical trials as a direct immunomodulatory agent. ALT-803 exhibits >25-fold enhancement in biological activity as compared to IL-15.

In certain embodiments, the cytokine is NIZ985 (hetIL-15). Studies have demonstrated that hetIL-15 administration can promote an increase of tumor infiltration and persistence of CD8+ T cells, including tumor-specific T cells, and result in an increased CD8+/Treg ratio. Tumor-resident CD8+ T cells show features of effector cells and are characterized by increased proliferation (Ki67+) and high cytotoxic potential (Granzyme B+). In the absence of hetIL-15, the smaller population of tumor-infiltrating T cells exhibit high levels of the exhaustion marker PD-1, potentially limiting their anti-cancer effectiveness. Provision of hetIL-15 can result in a significant decrease in lymphocyte expression of PD-1, alleviating one potential mechanism for the exhaustion phenotype. Preclinical cancer studies support the use of hetIL-15 in tumor immunotherapy approaches to promote the development of anti-tumor responses by favoring effector over regulatory cells.

In certain embodiments, the cytokine is interferon α (IFN-α). The IFN-α proteins are produced by leukocytes. They are mainly involved in innate immune response against viral infection.

In certain embodiments, the cytokine is interferon β (IFN-β). IFN-β comprises proteins produced by fibroblasts and is involved in innate immune response. IFN-β stimulates both macrophages and NK cells to elicit an anti-viral response, and are also active against tumors. In mice, IFN-β inhibits immune cells to produce growth factors, thereby slowing tumor growth, and inhibits other cells from producing vessel producing growth factors, thereby blocking tumor angiogenesis and hindering the tumor from connecting into the blood vessel system.

In certain embodiments, the cytokine is interferon γ (IFN-γ). IFN-γ, or type II interferon, is a cytokine that is useful for innate and adaptive immunity. IFN-γ is an important activator of macrophages and inducer of Class II major histocompatibility complex (MHC) molecule expression. The in vitro study of IFN-γ in cancer cells is extensive and results indicate anti-proliferative activity of IFN-γ leading to growth inhibition or cell death, generally induced by apoptosis but sometimes by autophagy. Clinical administration of IFN-γ has resulted in improved survival for patients with ovarian, bladder, and melanoma cancers.

In certain embodiments, the cytokine is a chemokine. Chemokines are a family of small cytokines. The major role of chemokines is to act as a chemoattractant to guide the migration of cells. Some chemokines control cells of the immune system during processes of immune surveillance, such as directing lymphocytes to the lymph nodes so they can screen for invasion of pathogens by interacting with antigen-presenting cells residing in these tissues. These are known as homeostatic chemokines and are produced and secreted without any need to stimulate their source cell(s). Some chemokines play a role in development, promote angiogenesis (the growth of new blood vessels), or guide cells to tissues that provide specific signals critical for cellular maturation. Other chemokines are inflammatory and are released from a wide variety of cells in response to bacteria, viruses, and agents that cause physical damage, such as silica or the urate crystals that occur in gout. Their release is often stimulated by proinflammatory cytokines, such as interleukin 1. Inflammatory chemokines function mainly as chemoattractants for leukocytes, recruiting monocytes, neutrophils, and other effector cells from the blood to sites of infection or tissue damage. Certain inflammatory chemokines activate cells to initiate an immune response or promote wound healing. They are released by many different cell types and serve to guide cells of both the innate immune system and adaptive immune system.

Furthermore, it is currently known in the art that the method of delivery, dosing and scheduling, and toxicity-related issues must be addressed to enable the immune-stimulating function of many chemokines to be fully exploited.

In certain embodiments, the chemokine is CCL1, CCL2, CCL3, CCL4, CCL5, CCL17, CCL19, CCL21, CCL22, CXCL9, CXCL10, CXCL11, CXCL13, CXCL16, or CX3CL1.

### Activator of Adaptive Immune Response

The drug delivery compositions and devices may comprise one or more activators of adaptive immune response.

The adaptive immune response system, also known as the acquired immune system, is a subsystem of the overall immune system that includes highly specialized systemic cells and processes that eliminate or prevent pathogen growth. The adaptive immune system is one of the two main immunity strategies found in vertebrates (the other being the innate immune system). Adaptive immunity creates immunological memory after an initial response to a specific pathogen and leads to an enhanced response to subsequent encounters with that pathogen. This process of acquired immunity is the basis of vaccination. Like the innate system, the adaptive system includes both humoral immunity components and cell-mediated immunity components. Unlike the innate immune system, the adaptive immune system is highly specific to a particular pathogen.

The adaptive immune response system is triggered in vertebrates when a pathogen evades the innate immune response system, generates a threshold level of antigen, and generates “stranger” or “danger” signals activating dendritic cells. The major functions of the acquired immune system include recognition of specific “non-self” antigens in the presence of “self” during the process of antigen presentation; generation of responses that are tailored to eliminate specific pathogens or pathogen-infected cells; and development of immunological memory, in which pathogens are “remembered” through memory B cells and memory T cells.

Useful approaches to activating the adaptive immune response system (e.g., activating therapeutic antitumor immunity) include the blockade of immune checkpoints. Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. Tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. Cytotoxic T-lymphocyte-associated antigen 4 (CTLA-4) antibodies were the first of this class of immunotherapeutics to receive FDA approval (ipilimumab). Preliminary clinical findings with blockers of additional immune-checkpoint proteins, such as programmed cell death protein 1 (PD-1), indicate broad and diverse opportunities to enhance antitumor immunity with the potential to produce durable clinical responses.

PD-1, functioning as an immune checkpoint, plays an important role in down-regulating the immune system by preventing the activation of T cells, which in turn reduces autoimmunity and promotes self-tolerance. The inhibitory effect of PD-1 is accomplished through a dual mechanism of promoting apoptosis (programmed cell death) in antigen-specific T cells in lymph nodes while simultaneously reducing apoptosis in regulatory T cells (suppressor T cells). A new class of therapeutics that block PD-1, the PD-1 inhibitors (e.g., anti-PD-1 antibodies), activate the immune system to attack tumors and are therefore used to treat some types of cancer. In addition, antibodies of Programmed death-ligand 1 (PD-L1) provide a similar impact on activating the adaptive immune response as antibodies targeting PD-1. Accordingly, compositions and devices comprising anti-PD-L1 antibodies are expected to provide a similar therapeutic effect as those comprising anti-PD-1 antibodies.

In certain embodiments, the activator of adaptive immune response is a small molecule. In certain embodiments, the activator of adaptive immune response is a biologic. In certain embodiments, the biologic is a protein. In certain embodiments, the biologic is an antibody or fragment thereof. In certain embodiments, the biologic is a nucleic acid that encodes a protein.

In certain embodiments, the activator of adaptive immune response is an anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD3 antibody, an anti-CD27 antibody, an anti-CD28 antibody, an anti-CD28H antibody, an anti-CD30 antibody, an anti-CD39 antibody, an anti-CD40 antibody, an anti-CD43 antibody, an anti-CD47 antibody, an anti-CD48 antibody, an anti-CD70 antibody, an anti-CD73 antibody, an anti-CD96 antibody, an anti-CD123 antibody, an anti-CD155 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GALS antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-CEACAM1 antibody, an anti-CEACAM5 antibody, an anti-CEACAM6 antibody, an anti-MICA antibody, an anti-MICB antibody, an anti-NKG2D antibody, an anti-NKG2A antibody, an anti-A2AR antibody, an anti-05aR antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, an anti-Tie2 antibody, an anti-CCR4 antibody, or an anti-TRAIL-DR5 antibody.

In certain embodiments, the activator of adaptive immune response is a fragment of any of the antibodies listed above. In certain embodiments, the activator of adaptive immune response is a humanized form of any of the antibodies listed above. In certain embodiments, the activator of adaptive immune response is a single chain of any of the antibodies listed above. In certain embodiments, the activator of immune response is a multimeric form of any of the antibodies listed above (e.g., dimeric IgA molecules, pentavalent IgM molecules).

In certain embodiments, the activator of adaptive immune response is an anti-PD-1 antibody, an agonist anti-CD137 antibody, an agonist anti-CD40 antibody, an anti-CTLA-4 antibody, an anti-LAG-3 antibody, an anti-TIM3, or a combination thereof. In certain embodiments, the activator of adaptive immune response is an anti-PD-1 antibody or an anti-CTLA-4 antibody. In certain embodiments, the activator of adaptive immune response is an anti-PD-1 antibody. In certain embodiments, the activator of adaptive immune response is an anti-CTLA-4 antibody. In certain embodiments, the activator of adaptive immune response is an agonist anti-CD137 antibody. In certain embodiments, the activator of adaptive immune response is an anti-LAG-3 antibody. In certain embodiments, the activator of adaptive immune response is an anti-TIM3 antibody.

In certain embodiments, the activator of adaptive immune response is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, IPH5401, IPH4102, IPH4301, IPH52, IPH53, varlilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, IMC-CS4, emactuzumab, trastuzumab, pertuzumab, obinutuzumab, cabiralizumab, margetuximab, enoblituzumab, mogamulizumab, panitumumab, carlumab, ramucirumab, bevacizumab, rituximab, cetuximab, fresolimumab, denosumab, MGA012, AGEN1884, AGEN2034, LY3300054, JTX-4014, teplizumab, FPA150, PF-04136309, PF-06747143, AZD5069, GSK3359609, FAZ053, TSR022, MBG453, REGN2810, REGN3767, MOXR0916, PF-04518600, R07009789, BMS986156, GWN323, JTX-2011, NKTR-214, GSK3174998, DS-8273a, NIS793, or BGB-A317.

In certain embodiments, the activator of adaptive immune response is pembrolizumab, nivolumab, pidilizumab, ipilimumab, tremelimumab, durvalumab, atezolizumab, REGN2810, MGA012, AGEN1884, AGEN2034, LY3300054, JTX-4014, or avelumab.

In certain embodiments, the activator of adaptive immune response is an antibody mimetic or antibody fusion.

In certain embodiments, the activator of adaptive immune response is a bispecific antibody. In certain embodiments, the bispecific antibody is RG7802 (antibody targeting carcinoembryonic antigen (CEA) and the CD3 receptor), RG7828 (a bispecific monoclonal antibody that targets CD20 on B cells and CD3 on T cells), RG7221 (a bispecific monoclonal antibody that targets VEGF and angiopoietin 2), RG7386 (a bispecific monoclonal antibody that targets FAP and DR5), ERY974 (a bispecific monoclonal antibody that targets CD3 and glypican-3), MGD012 (a bispecific monoclonal antibody that targets PD-1 and LAG-3), AMG211 (a bispecific T cell engager that targets CD3 and CEA), MEDI573 (a bispecific monoclonal antibody that targets IGF1 and IGF2), MEDI565 (a bispecific monoclonal antibody that targets CD3 and CEA), FS17 (undisclosed targets), FS18 (a bispecific monoclonal antibody that targets LAG3 and an undisclosed target), FS20 (undisclosed targets), FS22 (undisclosed targets), FS101 (a bispecific monoclonal antibody that targets EGFR and HGF), FS117 (undisclosed targets), FS118 (a bispecific monoclonal antibody that targets LAG3 and PD-L1), R06958688 (a bispecific monoclonal antibody that targets CD3 and CEA), MCLA-128 (a bispecific monoclonal antibody that targets HER2 and HER3), M7824 (bi-functional fusion-protein targeting PD-L1 and TGFβ), MGD009 (a humanized antibody that recognizes both B7-H3 and CD3), or MGD013 (a bispecific PD-1 and LAG-3 antibody).

In certain embodiments, the activator of adaptive immune response is an antibody-drug conjugate. In certain embodiments, the antibody-drug conjugate is trastuzumab emtansine, inotuzumab ozogamicin, PF-06647020, PF-06647263, PF-06650808, RG7596, RG7841, RG7882, RG7986, DS-8201, ABBV-399, glembatumumab vedotin, inotuzumab ozogamicin, MEDI4276, or pharmaceutically acceptable salts thereof.

In certain embodiments, the activator of adaptive immune response is a small molecule. In certain embodiments, the small molecule is an IDO inhibitor, a TGFβR inhibitor, a BRAF inhibitor, a KIT inhibitor, an A2aR inhibitor, a Tie2 inhibitor, an arginase inhibitor, an iNOS inhibitor, an HIF1α inhibitor, a STAT3 inhibitor, a PGE2 inhibitor, a PDE5 inhibitor, a RON inhibitor, an mTOR inhibitor, a JAK2 inhibitor, a HSP90 inhibitor, a PI3K-AKT inhibitor, a β-catenin inhibitor, a GSK3β inhibitor, an IAP inhibitor, an HDAC inhibitor, a DNMT inhibitor, a BET inhibitor, a COX2 inhibitor, a PDGFR inhibitor, a VEGFR inhibitor, a BCR-ABL inhibitor, a proteasome inhibitor, an angiogenesis inhibitor, a MEK inhibitor, a BRAF+MEK inhibitor, a pan-RAF inhibitor, an EGFR inhibitor, a PARP inhibitor, a glutaminase inhibitor, a WNT inhibitor, a FAK inhibitor, an ALK inhibitor, a CDK4/6 inhibitor, or an FGFR3 inhibitor.

In certain embodiments, the small molecule is celecoxib, sunitinib, imatinib, vemurafenib, dabrafenib, bortezomib, vorinostat, pomalidomide, thalidomide, lenalidomide, epacadostat, indoximid, GDC0919, BMS986205, AZD8055, AZD4635, CPI-444, PBF509, LCL161, CB-839, CB-1158, FPA008, BLZ945, IPI-549, pexidartinib, galunisertib, birinapant, trametinib, cobimetinib, binimetinib, ensartib, gefitinib, pazopanib, sorafenib, nintedanib, SYM004, veliparib, olaparib, BGB-290, everolimus, LXH254, azacitidine, decitabine, guadecitabine, RRX001, CC486, romidepsin, entinostat, panobinostat, tamoxifen, ibrutinib, idelalisib, capmatinib, selumetinib, abemaciclib, palbociclib, glasdegib, enzalutamide, AZD9150, PF-06840003, SRF231, Hu5F9-G4, CC-900002, TTI-621, WNT974, BGJ398, LY2874455, or pharmaceutically acceptable salts thereof.

### Additional Therapeutic Agents

The drug delivery compositions and devices may comprise additional therapeutic agents.

In certain embodiments, the drug delivery compositions and devices may comprise a modulator of macrophage effector function. Macrophages are immune cells that are derived from circulating monocytes, reside in all tissues, and participate in many states of pathology. Macrophages play a dichotomous role in cancer, where they can promote tumor growth but also can serve as critical immune effectors of therapeutic antibodies. Macrophages express all classes of Fcγ receptors, and they have potential to destroy tumors via the process of antibody-dependent cellular phagocytosis. A number of studies have demonstrated that macrophage phagocytosis is a major mechanism of action of many antibodies approved to treat cancer. Consequently, a number of approaches to augment macrophage responses to therapeutic antibodies are under investigation, including the exploration of new targets and development of antibodies with enhanced functions. The response of macrophages to antibody therapies can also be enhanced with engineered Fc variants, bispecific antibodies, or antibody-drug conjugates. Macrophages have demonstrated success as effectors of cancer immunotherapy.

In certain embodiments, the modulator of macrophage effector function is a modulator of suppressive myeloid cells, including myeloid-derived suppressor cells (MDSCs). In certain embodiments, the modulator of macrophage effector function may kill, deplete, or potentiate macrophages and/or MDSCs. In certain embodiments, the modulator of macrophage effector function is an anti-CD40 antibody, an anti-CD47 antibody, an anti-CSF1 antibody, or an anti-CSF1R antibody. In certain embodiments, the modulator of macrophage effector function is SRF231, Hu5F9-G4, CC-900002, or TTI-621 (anti-CD47 antibodies). In certain embodiments, the modulator of macrophage effector function is MCS-110 (an anti-CSF1 antibody). In certain embodiments, the modulator of macrophage effector function is FPA008, RG7155, IMC-CS4, AMG820, or UCB6352 (anti-CSF1R antibodies). In certain embodiments, the modulator of macrophage effector function is a small molecule inhibitor of CSF1R. In certain embodiments, the modulator of macrophage effector function is BLZ945, GW2580, or PLX3397 (small molecule inhibitors of CSF1R). In certain embodiments, the modulator of macrophage effector function is a BTK inhibitor, an ITK inhibitor, a PI3Kγ inhibitor, or a PI3Kδ inhibitor. In certain embodiments, the modulator of macrophage effector function may replace one or more activators of adaptive immune response in the composition or device.

In certain embodiments, the drug delivery compositions and devices may further comprise an oncolytic virus. In certain embodiments, the oncolytic virus includes, but is not limited to, herpes simplex viruses (e.g., HSV1716, OncoVex GM-CSF); adenoviruses (e.g., H101, Onyx-15); polioviruses (e.g., PV1(RIPO)); reoviruses (e.g., reolysin); senecaviruses (e.g., NTX-010, SVV-001); Rigvir virus; maraba virus; measles; Newcastle disease virus; vaccinia; or ECHO virus.

In certain embodiments, the drug delivery compositions and devices may further comprise a radioactive isotope (e.g., as part of a molecule or on a bead). In certain embodiments the radioactive isotope is Yttrium-90, Palladium-103, Iodine-125, Cesium 131, or Iridium 192.

In certain embodiments, the drug delivery compositions and devices may further comprise a chemotherapeutic agent. In certain embodiments, the chemotherapeutic agent includes, but is not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine and temozolomide), platinum-containing compounds (e.g., cisplatin, carboplatin, and oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, and mytomycin C), anti-metabolites, DHFR inhibitors (e.g., methotrexate, dichloromethotrexate, trimetrexate, and edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, and capecitabine), cytosine analogs (e.g., cytarabine (ara C), cytosine arabinoside, and fludarabine), purine analogs (e.g., mercaptopurine and thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, and peplomycin), anthracycline (e.g., daunorubicin, doxorubicin, pegylated liposomal doxorubicin, idarubicin, epirubicin, pirarubicin, zorubicin, and mitoxantrone), MDR inhibitors (e.g., verapamil), Ca²⁺ ATPase inhibitors (e.g., thapsigargin), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, hexamethyl melamine, and pharmaceutically acceptable salts thereof.

In certain embodiments, the chemotherapeutic agent is an immunomodulatory chemotherapeutic agent. In certain embodiments, the chemotherapeutic agent has known immunomodulatory function (e.g., induction of immunogenic cell death or depletion of immunosuppressive regulatory immune cells). In certain embodiments, the chemotherapeutic agent is included in the drug delivery compositions and devices due to its immunotherapeutic properties rather than its use as a conventional cancer-cell intrinsic cytotoxic chemotherapy. In certain embodiments, the drug delivery compositions and devices do not comprise a chemotherapeutic agent. In certain embodiments, the drug delivery compositions and devices do not comprise a cytotoxic agent.

In certain embodiments, the drug delivery compositions and devices may further comprise a targeted agent. In certain embodiments, the targeted agent includes, but is not limited to, an IDO inhibitor, a TGFβR inhibitor, an arginase inhibitor, an iNOS inhibitor, a HIF1α inhibitor, a STAT3 inhibitor, a CSF1R inhibitor, a PGE2 inhibitor, a PDE5 inhibitor, a RON inhibitor, an mTOR inhibitor, a JAK2 inhibitor, an HSP90 inhibitor, a PI3K-AKT inhibitor, a β-catenin inhibitor, a GSK3β inhibitor, an IAP inhibitor, an HDAC inhibitor, a DNMT inhibitor, a BET inhibitor, an A2AR inhibitor, a BRAF+MEK inhibitor, a pan-RAF inhibitor, a PI3Kγ inhibitor, a PI3Kδ inhibitor, an EGFR inhibitor, a VEGF inhibitor, a PARP inhibitor, a glutaminase inhibitor, a BTK inhibitor, an ITK inhibitor, a WNT inhibitor, a FAK inhibitor, an ALK inhibitor, a CDK4/6 inhibitor, a or an FGFR3 inhibitor.

In certain embodiments, the targeted agent includes, but is not limited to, imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), epacadostat, indoximid, GDC0919, BMS986205, AZD4635, CPI-444, PBF509, LCL161, CB-839, CB-1158, FPA008, BLZ945, IPI-549, pexidartinib, galunisertib, birinapant, trametinib, dabrafenib, vemurafenib, cobimetinib, binimetinib, ensartib, pazopanib, nintedanib, SYM004, veliparib, olaparib, BGB-290, LXH254, azacitidine, decitabine, guadecitabine, RRX001, CC486, romidepsin, entinostat, vorinostat, panobinostat, tamoxifen, ibrutinib, idelalisib, capmatinib, selumetinib, abemaciclib, palbociclib, glasdegib, enzalutamide, AZD9150, PF-06840003, SRF231, Hu5F9-G4, CC-900002, TTI-621, WNT974, BGJ398, LY2874455, an anti-Tie2 antibody, or pharmaceutically acceptable salts thereof.

### Embodiments of the Drug Delivery Compositions and Devices

In certain embodiments, the drug delivery compositions and devices comprise a hydrogel and an inhibitor of a proinflammatory pathway.

In certain embodiments, the drug delivery compositions and devices comprise a hydrogel, an inhibitor of a proinflammatory pathway, and an activator of innate immune response.

In certain embodiments, the drug delivery compositions and devices comprise a hydrogel, an inhibitor of a proinflammatory pathway, an activator of innate immune response, and an additional activator of innate immune response.

In certain embodiments, the drug delivery compositions and devices comprise a hydrogel, an inhibitor of a proinflammatory pathway, an activator of innate immune response, and a cytokine.

In certain embodiments, the drug delivery compositions and devices comprise a hydrogel, an inhibitor of a proinflammatory pathway, an activator of innate immune response, an additional activator of innate immune response, and a cytokine.

In certain embodiments, the drug delivery compositions and devices comprise a hydrogel, an inhibitor of a proinflammatory pathway, an activator of innate immune response, and a chemokine.

In certain embodiments, the drug delivery compositions and devices comprise a hydrogel, an inhibitor of a proinflammatory pathway, an activator of innate immune response, an additional activator of innate immune response, and a chemokine.

In certain embodiments, the drug delivery compositions and devices comprise a hydrogel, an inhibitor of a proinflammatory pathway, and a cytokine.

In certain embodiments, the drug delivery compositions and devices comprise a hydrogel, an inhibitor of a proinflammatory pathway, and a chemokine.

In certain embodiments, the drug delivery compositions and devices comprise a hydrogel, an inhibitor of a proinflammatory pathway, and an activator of adaptive immune response.

In certain embodiments, the drug delivery compositions and devices comprise a hydrogel, an inhibitor of a proinflammatory pathway, an activator of innate immune response, and an activator of adaptive immune response.

In certain embodiments, the drug delivery compositions and devices comprise a hydrogel, an inhibitor of a proinflammatory pathway, an activator of innate immune response, an additional activator of innate immune response, and an activator of adaptive immune response.

In certain embodiments, the drug delivery compositions and devices comprise a hydrogel, an inhibitor of a proinflammatory pathway, an activator of adaptive immune response, and an additional activator of adaptive immune response.

In certain embodiments, the drug delivery compositions and devices comprise a hydrogel, an inhibitor of a proinflammatory pathway, an activator of adaptive immune response, and two additional activators of the adaptive immune response.

In certain embodiments, the drug delivery compositions and devices comprise a hydrogel, an inhibitor of a proinflammatory pathway, an activator of innate immune response, a cytokine, and an activator of adaptive immune response.

In certain embodiments, the drug delivery compositions and devices comprise a hydrogel, an inhibitor of a proinflammatory pathway, an activator of innate immune response, an additional activator of innate immune response, a cytokine, and an activator of adaptive immune response.

In certain embodiments, the drug delivery compositions and devices comprise a hydrogel, an inhibitor of a proinflammatory pathway, an activator of innate immune response, a cytokine, an activator of adaptive immune response, and an additional activator of adaptive immune response.

In certain embodiments, the drug delivery compositions and devices comprise a hydrogel, an inhibitor of a proinflammatory pathway, an activator of innate immune response, an additional activator of innate immune response, a cytokine, an activator of adaptive immune response, and an additional activator of adaptive immune response.

In certain embodiments, the drug delivery compositions and devices comprise a hydrogel, an inhibitor of a proinflammatory pathway, an activator of innate immune response, a chemokine, and an activator of adaptive immune response.

In certain embodiments, the drug delivery compositions and devices comprise a hydrogel, an inhibitor of a proinflammatory pathway, an activator of innate immune response, an additional activator of innate immune response, a chemokine, and an activator of adaptive immune response.

In certain embodiments, the drug delivery compositions and devices comprise a hydrogel, an inhibitor of a proinflammatory pathway, an activator of innate immune response, a chemokine, an activator of adaptive immune response, and an additional activator of adaptive immune response.

In certain embodiments, the drug delivery compositions and devices comprise a hydrogel, an inhibitor of a proinflammatory pathway, an activator of innate immune response, an additional activator of innate immune response, a chemokine, an activator of adaptive immune response, and an additional activator of adaptive immune response.

In certain embodiments, the drug delivery compositions and devices comprise a hydrogel, an inhibitor of a proinflammatory pathway, a cytokine, and an activator of adaptive immune response.

In certain embodiments, the drug delivery compositions and devices comprise a hydrogel, an inhibitor of a proinflammatory pathway, a cytokine, an activator of adaptive immune response, and an additional activator of adaptive immune response.

In certain embodiments, the drug delivery compositions and devices comprise a hydrogel, an inhibitor of a proinflammatory pathway, a chemokine, and an activator of adaptive immune response.

In certain embodiments, the drug delivery compositions and devices comprise a hydrogel, an inhibitor of a proinflammatory pathway, a chemokine, an activator of adaptive immune response, and an additional activator of adaptive immune response.

In certain embodiments, the drug delivery compositions and devices comprise a hydrogel and an anti-IL-1β antibody.

In certain embodiments, the drug delivery compositions and devices comprise a hydrogel and an anti-IL-6 antibody.

In certain embodiments, the drug delivery compositions and devices comprise a hydrogel and an anti-IL-6R antibody.

In certain embodiments, the drug delivery compositions and devices comprise a hydrogel and an inhibitor of a proinflammatory immune response mediated by a p38 mitogen-activated protein kinase (MAPK) pathway.

In certain embodiments, the drug delivery compositions and devices comprise a hydrogel and a p38 MAPK inhibitor.

In certain embodiments, the drug delivery compositions and devices comprise a hydrogel and a p38 α/β MAPK inhibitor that binds to an ATP and/or allosteric binding site of p38 MAPK.

In certain embodiments, the drug delivery compositions and devices comprise a hydrogel and losmapimod.

In certain embodiments, the drug delivery compositions and devices comprise a hydrogel and a TGFβR inhibitor.

In certain embodiments, the drug delivery compositions and devices comprise a hydrogel and a CCR2 inhibitor.

In certain embodiments, the drug delivery compositions and devices comprise a hydrogel and a CXCR4 inhibitor.

In certain embodiments, the drug delivery compositions and devices comprise a hydrogel, an anti-IL-1β antibody, and a stimulator of interferon genes (STING) agonist.

In certain embodiments, the drug delivery compositions and devices comprise a hydrogel, an anti-IL-6 antibody, and a stimulator of interferon genes (STING) agonist.

In certain embodiments, the drug delivery compositions and devices comprise a hydrogel, an anti-IL-6R antibody, and a stimulator of interferon genes (STING) agonist.

In certain embodiments, the drug delivery compositions and devices comprise a hydrogel, a TGFβR inhibitor, and a stimulator of interferon genes (STING) agonist.

In certain embodiments, the drug delivery compositions and devices comprise a hydrogel, a CCR2 inhibitor, and a stimulator of interferon genes (STING) agonist.

In certain embodiments, the drug delivery compositions and devices comprise a hydrogel, a CXCR4 inhibitor, and a stimulator of interferon genes (STING) agonist.

In certain embodiments, the drug delivery compositions and devices comprise a hydrogel, an inhibitor of a proinflammatory immune response mediated by a p38 mitogen-activated protein kinase (MAPK) pathway, and a TLR7/8 agonist.

In certain embodiments, the drug delivery compositions and devices comprise a hydrogel, a p38 MAPK inhibitor, and a TLR7/8 agonist.

In certain embodiments, the drug delivery compositions and devices comprise a hydrogel, a p38 α/β MAPK inhibitor that binds to an ATP and/or allosteric binding site of p38 MAPK, and a TLR7/8 agonist.

In certain embodiments, the drug delivery compositions and devices comprise a hydrogel, losmapimod, and a TLR7/8 agonist.

In certain embodiments, the drug delivery compositions and devices comprise a hydrogel, an anti-IL-1β antibody, and 2′3′-cGAMP.

In certain embodiments, the drug delivery compositions and devices comprise a hydrogel, an anti-IL-6 antibody, and 2′3′-cGAMP.

In certain embodiments, the drug delivery compositions and devices comprise a hydrogel, an anti-IL-6R antibody, and 2′3′-cGAMP.

In certain embodiments, the drug delivery compositions and devices comprise a hydrogel, an anti-IL-1β antibody, and 2′3′-c-di-AM(PS)2 (Rp,Rp).

In certain embodiments, the drug delivery compositions and devices comprise a hydrogel, an anti-IL-6 antibody, and 2′3′-c-di-AM(PS)2 (Rp,Rp).

In certain embodiments, the drug delivery compositions and devices comprise a hydrogel, an anti-IL-6R antibody, and 2′3′-c-di-AM(PS)2 (Rp,Rp).

In certain embodiments, the drug delivery compositions and devices comprise a hydrogel, an inhibitor of a proinflammatory immune response mediated by a p38 mitogen-activated protein kinase (MAPK) pathway, and resiquimod.

In certain embodiments, the drug delivery compositions and devices comprise a hydrogel, a p38 MAPK inhibitor, and resiquimod.

In certain embodiments, the drug delivery compositions and devices comprise a hydrogel, a p38 α/β MAPK inhibitor that binds to an ATP and/or allosteric binding site of p38 MAPK, and resiquimod.

In certain embodiments, the drug delivery compositions and devices comprise a hydrogel, losmapimod, and resiquimod.

In certain embodiments, the drug delivery compositions and devices comprise a hydrogel and resiquimod.

In certain embodiments, the drug delivery compositions and devices comprise hyaluronic acid and an anti-IL-1β antibody.

In certain embodiments, the drug delivery compositions and devices comprise hyaluronic acid and an anti-IL-6 antibody.

In certain embodiments, the drug delivery compositions and devices comprise hyaluronic acid and an anti-IL-6R antibody.

In certain embodiments, the drug delivery compositions and devices comprise hyaluronic acid and an inhibitor of a proinflammatory immune response mediated by a p38 mitogen-activated protein kinase (MAPK) pathway.

In certain embodiments, the drug delivery compositions and devices comprise hyaluronic acid and a p38 MAPK inhibitor.

In certain embodiments, the drug delivery compositions and devices comprise hyaluronic acid and a p38 α/β MAPK inhibitor that binds to an ATP and/or allosteric binding site of p38 MAPK.

In certain embodiments, the drug delivery compositions and devices comprise hyaluronic acid and losmapimod.

In certain embodiments, the drug delivery compositions and devices comprise hyaluronic acid and a TGFβR inhibitor.

In certain embodiments, the drug delivery compositions and devices comprise hyaluronic acid and a CCR2 inhibitor.

In certain embodiments, the drug delivery compositions and devices comprise hyaluronic acid and a CXCR4 inhibitor.

In certain embodiments, the drug delivery compositions and devices comprise hyaluronic acid, an anti-IL-1β antibody, and a stimulator of interferon genes (STING) agonist.

In certain embodiments, the drug delivery compositions and devices comprise hyaluronic acid, an anti-IL-6 antibody, and a stimulator of interferon genes (STING) agonist.

In certain embodiments, the drug delivery compositions and devices comprise hyaluronic acid, an anti-IL-6R antibody, and a stimulator of interferon genes (STING) agonist.

In certain embodiments, the drug delivery compositions and devices comprise hyaluronic acid, a TGFβR inhibitor, and a stimulator of interferon genes (STING) agonist.

In certain embodiments, the drug delivery compositions and devices comprise hyaluronic acid, a CCR2 inhibitor, and a stimulator of interferon genes (STING) agonist.

In certain embodiments, the drug delivery compositions and devices comprise hyaluronic acid, a CXCR4 inhibitor, and a stimulator of interferon genes (STING) agonist.

In certain embodiments, the drug delivery compositions and devices comprise hyaluronic acid, an inhibitor of a proinflammatory immune response mediated by a p38 mitogen-activated protein kinase (MAPK) pathway, and a TLR7/8 agonist.

In certain embodiments, the drug delivery compositions and devices comprise hyaluronic acid, a p38 MAPK inhibitor, and a TLR7/8 agonist.

In certain embodiments, the drug delivery compositions and devices comprise hyaluronic acid, a p38 α/β MAPK inhibitor that binds to an ATP and/or allosteric binding site of p38 MAPK, and a TLR7/8 agonist.

In certain embodiments, the drug delivery compositions and devices comprise hyaluronic acid, losmapimod, and a TLR7/8 agonist.

In certain embodiments, the drug delivery compositions and devices comprise hyaluronic acid, an anti-IL-1β antibody, and 2′3′-cGAMP.

In certain embodiments, the drug delivery compositions and devices comprise hyaluronic acid, an anti-IL-6 antibody, and 2′3′-cGAMP.

In certain embodiments, the drug delivery compositions and devices comprise hyaluronic acid, an anti-IL-6R antibody, and 2′3′-cGAMP.

In certain embodiments, the drug delivery compositions and devices comprise hyaluronic acid, an anti-IL-1β antibody, and 2′3′-c-di-AM(PS)2 (Rp,Rp).

In certain embodiments, the drug delivery compositions and devices comprise hyaluronic acid, an anti-IL-6 antibody, and 2′3′-c-di-AM(PS)2 (Rp,Rp).

In certain embodiments, the drug delivery compositions and devices comprise hyaluronic acid, an anti-IL-6R antibody, and 2′3′-c-di-AM(PS)2 (Rp,Rp).

In certain embodiments, the drug delivery compositions and devices comprise hyaluronic acid, an inhibitor of a proinflammatory immune response mediated by a p38 mitogen-activated protein kinase (MAPK) pathway, and resiquimod.

In certain embodiments, the drug delivery compositions and devices comprise hyaluronic acid, a p38 MAPK inhibitor, and resiquimod.

In certain embodiments, the drug delivery compositions and devices comprise hyaluronic acid, a p38 α/β MAPK inhibitor that binds to an ATP and/or allosteric binding site of p38 MAPK, and resiquimod.

In certain embodiments, the drug delivery compositions and devices comprise hyaluronic acid, losmapimod, and resiquimod.

In certain embodiments, the drug delivery compositions and devices comprise a hyaluronic acid and resiquimod.

In certain embodiments, the drug delivery compositions and devices comprise alginate and an anti-IL-1β antibody.

In certain embodiments, the drug delivery compositions and devices comprise alginate and an anti-IL-6 antibody.

In certain embodiments, the drug delivery compositions and devices comprise alginate and an anti-IL-6R antibody.

In certain embodiments, the drug delivery compositions and devices comprise alginate and an inhibitor of a proinflammatory immune response mediated by a p38 mitogen-activated protein kinase (MAPK) pathway.

In certain embodiments, the drug delivery compositions and devices comprise alginate and a p38 MAPK inhibitor.

In certain embodiments, the drug delivery compositions and devices comprise alginate and a p38 α/β MAPK inhibitor that binds to an ATP and/or allosteric binding site of p38 MAPK.

In certain embodiments, the drug delivery compositions and devices comprise alginate and losmapimod.

In certain embodiments, the drug delivery compositions and devices comprise alginate, an anti-IL-1β antibody, and a stimulator of interferon genes (STING) agonist.

In certain embodiments, the drug delivery compositions and devices comprise alginate, an anti-IL-6 antibody, and a stimulator of interferon genes (STING) agonist.

In certain embodiments, the drug delivery compositions and devices comprise alginate, an anti-IL-6R antibody, and a stimulator of interferon genes (STING) agonist.

In certain embodiments, the drug delivery compositions and devices comprise alginate, an inhibitor of a proinflammatory immune response mediated by a p38 mitogen-activated protein kinase (MAPK) pathway, and a TLR7/8 agonist.

In certain embodiments, the drug delivery compositions and devices comprise alginate, a p38 MAPK inhibitor, and a TLR7/8 agonist.

In certain embodiments, the drug delivery compositions and devices comprise alginate, a p38 α/β MAPK inhibitor that binds to an ATP and/or allosteric binding site of p38 MAPK, and a TLR7/8 agonist.

In certain embodiments, the drug delivery compositions and devices comprise alginate, losmapimod, and a TLR7/8 agonist.

In certain embodiments, the drug delivery compositions and devices comprise alginate, an anti-IL-1β antibody, and 2′3′-cGAMP.

In certain embodiments, the drug delivery compositions and devices comprise alginate, an anti-IL-6 antibody, and 2′3′-cGAMP.

In certain embodiments, the drug delivery compositions and devices comprise alginate, an anti-IL-6R antibody, and 2′3′-cGAMP.

In certain embodiments, the drug delivery compositions and devices comprise alginate, an anti-IL-1β antibody, and 2′3′-c-di-AM(PS)2 (Rp,Rp).

In certain embodiments, the drug delivery compositions and devices comprise alginate, an anti-IL-6 antibody, and 2′3′-c-di-AM(PS)2 (Rp,Rp).

In certain embodiments, the drug delivery compositions and devices comprise alginate, an anti-IL-6R antibody, and 2′3′-c-di-AM(PS)2 (Rp,Rp).

In certain embodiments, the drug delivery compositions and devices comprise alginate, an inhibitor of a proinflammatory immune response mediated by a p38 mitogen-activated protein kinase (MAPK) pathway, and resiquimod.

In certain embodiments, the drug delivery compositions and devices comprise alginate, a p38 MAPK inhibitor, and resiquimod.

In certain embodiments, the drug delivery compositions and devices comprise alginate, a p38 α/β MAPK inhibitor that binds to an ATP and/or allosteric binding site of p38 MAPK, and resiquimod.

In certain embodiments, the drug delivery compositions and devices comprise alginate, losmapimod, and resiquimod.

In certain embodiments, the drug delivery compositions and devices comprise alginate and resiquimod.

In certain embodiments, the drug delivery compositions and devices do not comprise alginate, a COX-2 inhibitor (e.g., celecoxib), and an anti-PD-1 antibody.

In certain embodiments, the drug delivery compositions and devices do not comprise 1,3,-bis(2-chloroethyl)-1-nitrosourea (BCNU) and ethylene-vinyl acetate copolymer.

### Properties of the Drug Delivery Compositions and Devices

Biomaterials useful for drug delivery compositions and devices described herein are biocompatible. In some embodiments, biomaterials (e.g., hydrogel) are biodegradable. The drug delivery compositions and devices are able to be degraded, chemically and/or biologically, within a physiological environment, such as within the body. Degradation of the compositions and devices may occur at varying rates, depending on the components and hydrogel used. For example, the half-life of the compositions and devices (the time at which 50% of the composition is degraded into monomers and/or other non-polymeric moieties) may be on the order of days, weeks, months, or years. The compositions and devices may be biologically degraded, e.g., by enzymatic activity or cellular machinery, in some cases, for example, through exposure to a lysozyme (e.g., having relatively low pH), or by simple hydrolysis. In some cases, the compositions and devices may be broken down into monomers and/or other non-polymeric moieties that cells can either reuse or dispose of without significant toxic effect on the cells. The drug delivery compositions and devices are stable in vivo such that they deliver drug to the intended target in a suitable amount of time.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the device remains in vivo 12 months after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 6 months after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 5 months after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 4 months after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 3 months after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 2 months after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 1 month after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 1 week after administration (e.g., implantation) of the drug delivery composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of the composition remains in vivo 1 day after administration (e.g., implantation) of the drug delivery composition or device.

The storage modulus in a viscoelastic material measures the stored energy of the elastic portion of the material. Storage modulus may be measured with a rheometer. Measurements provided herein were made at room temperature with TA Instruments AR-G2 Magnetic Bearing Rheometer. The storage modulus of the drug delivery compositions and devices will vary based on the components of the composition.

Generally, the relationship between storage modulus and concentration of thiol-modified hyaluronic acid (e.g., GLYCOSIL®) and the thiol-reactive PEGDA cross-linker (e.g., EXTRALINK®) is linear (excluding the limits of sensitivity). For example, a formulation of 0.8% GLYCOSIL® and 0.2% EXTRALINK® will have a storage modulus of about 100 Pa, and a formulation of 1.3% GLYCOSIL® and 2% EXTRALINK® will have a storage modulus of about 1600 Pa.

In certain embodiments, a drug delivery composition or device described herein has a storage modulus of at least 50 Pa, at least 100 Pa, at least 200 Pa, at least 300 Pa, at least 400 Pa, at least 500 Pa, at least 600 Pa, at least 700 Pa, at least 800 Pa, at least 900 Pa, at least 1000 Pa, at least 1100 Pa, at least 1200 Pa, at least 1300 Pa, at least 1400 Pa, at least 1500 Pa, at least 1600 Pa, at least 1700 Pa, at least 1800 Pa, at least 1900 Pa, at least 2000 Pa, at least 2100 Pa, at least 2200 Pa, at least 2300 Pa, at least 2400 Pa, at least 2500 Pa, at least 2600 Pa, at least 2700 Pa, at least 2800 Pa, at least 2900 Pa, or at least 3000 Pa.

In certain embodiments, a drug delivery composition or device described herein has a storage modulus of about 50 Pa to about 100,000,000 Pa, about 50 Pa to about 100,000 Pa, about 50 Pa to about 10,000 Pa, about 50 Pa to about 3,000 Pa, about 100 Pa to about 3,000 Pa, about 100 Pa to about 2,000 Pa, about 500 Pa to about 3,000 Pa, about 500 Pa to about 2,000 Pa, about 1,000 Pa to about 2,000 Pa, about 1,200 Pa to about 1,800 Pa, about 1,300 Pa to about 1,700 Pa, or about 1,400 Pa to about 1,600 Pa.

In certain embodiments, a drug delivery composition or device described herein has a storage modulus of up to about 600 Pa, up to about 700 Pa, up to about 800 Pa, up to about 900 Pa, up to about 1,000 Pa, up to about 1,100 Pa, up to about 1,200 Pa, up to about 1,300 Pa, up to about 1,400 Pa, up to about 1,500 Pa, up to about 1,600 Pa, up to about 1,700 Pa, up to about 1,800 Pa, up to about 1,900 Pa, up to about 2,000 Pa, up to about 2,500 Pa, up to about 3,000 Pa, up to about 5,000 Pa, up to about 10,000 Pa, up to about 100,000 Pa, up to about 1,000,000 Pa, up to about 10,000,000 Pa, or up to about 100,000,000 Pa.

Drug delivery compositions and devices described herein release one or more therapeutic agents under physiological conditions, such as within the body. Release of one or more therapeutic agents may occur at varying rates, depending on the components of the composition or device (e.g., identity and concentration of the hydrogel). For example, the release rate of one or more therapeutic agents (the time at which the therapeutic agent(s) is/are no longer a part of the composition or device) may be on the order of minutes, hours, days, weeks, months, or years. Therapeutic agents may be released by various mechanisms, e.g., by diffusion, chemical activity, enzymatic activity, or cellular machinery. In some embodiments, drug delivery compositions and devices described herein are stable in vivo such that they deliver drug to an intended target in a suitable amount of time.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of the activator of the innate immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of the activator of the innate immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of any additional activator of the innate immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of any additional activator of the innate immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of the activator of the the adaptive immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of the activator of the adaptive immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of any additional activator of the adaptive immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of any additional activator of the adaptive immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of the cytokine is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of the cytokine is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation) of the composition or device.

### Preparation and Administration of the Drug Delivery Compositions and Devices

The present disclosure provides drug delivery compositions and devices comprising therapeutic agents, as described herein. In certain embodiments, the therapeutic agents are provided in an effective amount in the drug delivery compositions and devices to treat and/or prevent a disease (e.g., a proliferative disease, such as cancer). In certain embodiments, the effective amount is a therapeutically effective amount of a particular therapeutic agent. In certain embodiments, the effective amount is a prophylactically effective amount of a particular therapeutic agent.

The drug delivery compositions and devices described herein can be prepared by any method known in the art of pharmacology. In certain embodiments, such preparatory methods include the steps of adding a thiol-modified hyaluronic acid into a mold; adding an inhibitor of a proinflammatory pathway (e.g., an inhibitor of a proinflammatory immune response mediated by a p38 MAPK pathway); optionally adding an activator of adaptive immune response to the mold; optionally adding a chemokine or cytokine to the mold; optionally adding an activator of innate immune response to the mold; adding a cross-linking agent to the mold (e.g., a thiol-reactive PEGDA cross-linker); and allowing the mixture to stand for at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 90 minutes, at least 2 hours, at least 3 hours, at least 4 hours, at least 5 hours, or at least 6 hours for solidification.

In certain embodiments, the concentration of thiol-modified hyaluronic acid (e.g., GLYCOSIL®) used for the preparation of the hydrogel is, by weight/volume, about 1% to about 10%, about 1% to about 5%, about 1% to about 3%, or about 1.5% to about 2.5%; and the amount of thiol-reactive PEGDA cross-linker (e.g., EXTRALINK®) used for the preparation of the hydrogel is, by weight/volume, about 1% to about 20%, about 10% to about 20%, about 5% to about 15%, or about 10% to about 15%. In certain preferred embodiments, the concentration of thiol-modified hyaluronic acid is about 2% w/v and the concentration of thiol-reactive PEGDA cross-linker is about 12.5% w/v. In certain embodiments, the formulation of 2% thiol-modified hyaluronic acid and 12.5% provides a hydrogel with a storage modulus of about 1000 Pa to about 2000 Pa.

For the preparation of standard tissue engineering applications known in the art, the typical concentration of thiol-modified hyaluronic acid (e.g., GLYCOSIL®) is about 1% w/v and the typical concentration of thiol-reactive PEGDA cross-linker (e.g., EXTRALINK®) is about 1% w/v. Thus, the use of 2% w/v thiol-modified hyaluronic acid (e.g., GLYCOSIL®) and 12.5% w/v thiol-reactive PEGDA cross-linker (e.g., EXTRALINK®) provides an unexpectedly useful and advantageous biomaterial in the disclosed drug delivery compositions and devices.

Those skilled in the art will appreciate that other crosslinkers may be used at appropriate concentrations to form a hydrogel (e.g., a hyaluronic acid hydrogel). For example, in some embodiments, a hydrogel (e.g., a hyaluronic acid hydrogel) can be crosslinked by attaching thiols (e.g., EXTRACEL®, HYSTEM®), methacrylates, hexadecylamides (e.g., HYMOVIS®), and/or tyramines (e.g., CORGEL®). In some embodiments, a hydrogel (e.g., a hyaluronic acid hydrogel) can be crosslinked directly with formaldehyde (e.g., HYLAN-A®), divinylsulfone (DVS) (e.g., HYLAN-B®), 1,4-butanediol diglycidyl ether (BDDE) (e.g., RESTYLANE®), glutaraldehyde, and/or genipin (see, e.g., Khunmanee et al. “Crosslinking method of hyaluronic-based hydrogel for biomedical applications” J Tissue Eng. 8: 1-16 (2017)). In some embodiments, a hydrogel (e.g., a hyaluronic acid hydrogel) is crosslinked with divinylsulfone (DVS) (e.g., HYLAN-B®).

In certain embodiments, the concentration of the alginate used for the preparation of the hydrogel is, by weight/volume, about 0.5% to about 2.5%, about 0.75% to about 2.0%, or about 1.0% to about 1.5% alginate. In certain embodiments, the amount of 1 M calcium chloride cross-linker solution used for the preparation of the hydrogel is about 5 μL to 25 μL about 10 μL to 20 μL, or about 15 In certain embodiments, the payload of interest can be loaded in about 10 μL to 70 μL solvent (PBS or DMSO), 20 μL to 60 μL solvent (PBS or DMSO), about 30 μL to 50 μL solvent (PBS or DMSO), or about 40 μL solvent (PBS or DMSO).

The drug delivery compositions and devices may further comprise at least one excipient. In certain embodiments, the excipient is phosphate-buffered saline, tris(hydroxymethyl)aminomethane, sodium chloride, potassium chloride, calcium chloride, magnesium sulfate, sodium bicarbonate, sodium phosphate, potassium phosphate, calcium nitrate, glucose, lactose, trehalose, sucrose, or a combination thereof. In certain embodiments, the excipient is phosphate-buffered saline, tris(hydroxymethyl)aminomethane, sodium chloride, or a combination thereof. In certain embodiments, the excipient is phosphate-buffered saline.

In certain embodiments, the drug delivery compositions and devices do not include nanoparticles or microparticles. Nanoparticles include particles between 1 and 100 nm in size. Microparticles include particles between 0.1 and 100 μm in size. In certain embodiments, the drug delivery compositions and devices do not include silica microparticles, polyethylene microparticles, polystyrene microparticles, polyester microparticles, polyanhydride microparticles, polycaprolactone microparticles, polycarbonate microparticles, or polyhydroxybutyrate microparticles. In certain embodiments, the drug delivery compositions and devices do not include porous silica microparticles.

In certain embodiments, the drug delivery compositions and devices include one or more organic solvents. In certain embodiments, the drug delivery compositions and devices include dimethylsulfoxide (DMSO).

In certain embodiments, the drug delivery compositions and devices do not include organic solvent. In certain embodiments, organic solvents are not used in the preparation of the compositions or devices. In certain embodiments, the drug delivery compositions and devices are free of organic solvent. In certain embodiments, the drug delivery compositions and devices are substantially free of organic solvent. In certain embodiments, the drug delivery compositions and devices comprise, by weight, less than 10%, less than 5%, less than 4%, less than 3%, less than 2%, less than 1%, less than 0.5%, less than 0.1%, less than 0.01%, less than 0.001%, or less than 0.0001% of organic solvent. In certain embodiments, the drug delivery compositions and devices comprise, by weight, less than 1000 ppm, less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 1 ppm, less than 10 ppb, or less than 1 ppb of organic solvent. In certain embodiments, the drug delivery composition does not include dimethylsulfoxide (DMSO).

In certain embodiments, the drug delivery compositions comprise organic solvent. In certain embodiments, the organic solvent is cyclodextrin, methanol, ethanol, isopropanol, ethylene glycol, propylene glycol, or a combination thereof.

The drug delivery compositions and devices can be prepared, packaged, and/or sold in bulk, as a single unit dose, and/or as a plurality of single unit doses. A “unit dose” is a discrete amount of the composition or device comprising a predetermined amount of the therapeutic agents. The amount of the therapeutic agents is generally equal to the dosage of the therapeutic agents which would be administered to a subject and/or a convenient fraction of such a dosage, such as, for example, one-half, one-third, or one-quarter of such a dosage.

Relative amounts of the therapeutic agents, the excipient, and/or any additional ingredients in a composition or device of the disclosure will vary, depending upon the identity, size, and/or condition of the subject treated. By way of example, the composition or device may comprise between 0.1% and 99% (w/w), between 0.1% and 90% (w/w), between 0.1% and 80% (w/w), between 0.1% and 70% (w/w), between 1% and 50% (w/w), between 10% and 80% (w/w), between 10% and 90% (w/w), between 10% and 80% (w/w), between 20% and 80% (w/w), between 30% and 80% (w/w), between 30% and 70% (w/w), or between 40% and 60% (w/w), of the therapeutic agents.

Additional pharmaceutically acceptable excipients may be used in the manufacture of the provided drug delivery compositions and devices. These include inert diluents, dispersing and/or granulating agents, surface-active agents and/or emulsifiers, disintegrating agents, binding agents, preservatives, buffering agents, lubricating agents, and/or oils. Excipients such as cocoa butter and suppository waxes, coloring agents, and coating agents may also be present in the composition or device.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80)), polyoxyethylene esters (e.g., polyoxyethylene monostearate (MYRJ 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (BRIJ 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC F-68 (also known as Poloxamer-188), PLURONIC F-127 (also known as Poloxamer-407), cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS, PHENONIP, methylparaben, GERMALL 115, GERMABEN II, NEOLONE, KATHON, and EUXYL.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Although the descriptions of drug delivery compositions provided herein are principally directed to compositions which are suitable for administration to humans, it will be understood by the skilled artisan that such compositions are generally suitable for administration to animals of all sorts. Modification of drug delivery compositions suitable for administration to humans in order to render the compositions suitable for administration to various animals is well understood, and the ordinarily skilled veterinary pharmacologist can design and/or perform such modification with ordinary experimentation.

The drug delivery compositions and devices provided herein are typically formulated in a size (e.g., volume) and weight appropriate for the intended use (e.g., surgical implantation) for ease of administration. It will be understood, however, that the total amount of the composition or device of the present disclosure (e.g., number of devices implanted) will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; the drugs used in combination or coincidental with the specific active ingredient employed; and like factors well known in the medical arts.

The drug delivery compositions and devices provided herein can be administered by surgical implantation. For example, the drug delivery composition or device may be administered by surgical implantation in the void volume of a resected tumor. As a further example, the drug delivery composition or device may be administered by surgical implantation and affixed with a bioadhesive. In certain embodiments, the drug delivery composition or device is affixed with a bioadhesive in the void volume of a resected tumor.

In certain embodiments, the drug delivery composition or device is administered by surgical implantation at a site within 100 cm, 90 cm, 80 cm. 70 cm, 60 cm. 50 cm, 40 cm, 30 cm, 20 cm, 10 cm, 9 cm, 8 cm, 7 cm, 6 cm, 5 cm, 4 cm, 3 cm, 2 cm, 1 cm, 9 mm, 8 mm, 7 mm, 6 mm, 5 mm, 4 mm, 3 mm, 2 mm, or 1 mm of the void volume of a resected tumor. In certain embodiments, the void volume of a resected tumor is the void volume of a resected organ having a tumor (e.g., lung, kidney, pancreas, liver, colon, testes, ovary, breast, appendix, bladder). In certain embodiments, the void volume of a resected tumor is the void volume of a resected portion of an organ having a tumor (e.g., lung, kidney, pancreas, liver, colon, testes, ovary, breast, appendix, bladder).

In certain embodiments, precursor components of a hydrogel (e.g., hyaluronic acid) and cross-linking agent are administered separately to a subject (e.g., at the site of tumor resection), thus forming the drug delivery composition in vivo. In certain embodiments, precursor components of a hydrogel (e.g., hyaluronic acid) and cross-linking agent are administered sequentially. In certain embodiments, precursor components of a hydrogel (e.g., hyaluronic acid) and cross-linking agent are administered concurrently. In certain embodiments, precursor components of a hydrogel (e.g., hyaluronic acid) and cross-linking agent are administered as a mixture. In certain embodiments, the administration is via injection.

In certain embodiments, the alginate and cross-linking agent are administered separately to a subject (e.g., at the site of tumor resection), thus forming the drug delivery composition in vivo. In certain embodiments, the alginate and cross-linking agent are administered sequentially. In certain embodiments, the alginate and cross-linking agent are administered concurrently. In certain embodiments, the alginate and cross-linking agent are administered as a mixture. In certain embodiments, the administration is via injection.

The exact amount of the therapeutic agents required to achieve effective amounts will vary from subject to subject, depending, for example, on species, age, and general condition of a subject, severity of the side effects or disorder, identity of the particular agent(s), and the like.

In certain embodiments, an effective amount of the composition or device for administration to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg.

In certain embodiments, the composition or device may be at dosage levels sufficient to deliver about 0.001 mg/kg to about 100 mg/kg, about 0.01 mg/kg to about 50 mg/kg, about 0.1 mg/kg to about 40 mg/kg, about 0.5 mg/kg to about 30 mg/kg, about 0.01 mg/kg to about 10 mg/kg, about 0.1 mg/kg to about 10 mg/kg, or about 1 mg/kg to about 25 mg/kg, of subject body weight per day, of any of the therapeutic agents present in the composition, to obtain the desired therapeutic effect.

It will be appreciated that dose ranges as described herein provide guidance for the administration of the provided drug delivery compositions and devices to an adult. The amount to be administered to, for example, a child or an adolescent can be determined by a medical practitioner or person skilled in the art and can be lower or the same as that administered to an adult.

It will be also appreciated that compositions and devices, as described herein, can be administered in combination with one or more additional pharmaceutical agents. For example, the compositions and devices can be administered in combination with additional pharmaceutical agents that reduce and/or modify their metabolism, inhibit their excretion, and/or modify their distribution within the body. It will also be appreciated that the additional therapy employed may achieve a desired effect for the same disorder, and/or it may achieve different effects.

The compositions and devices can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents will be administered separately in different doses and/or different routes of administration. The particular combination to employ in a regimen will take into account compatibility of the drug delivery composition with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-inflammatory agents, immunosuppressant agents, and pain-relieving agents. Pharmaceutical agents include small molecule therapeutics such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

In certain embodiments, the drug delivery compositions and devices do not include cells. In certain embodiments, the drug delivery compositions and devices do not include adoptively transferred cells. In certain embodiments, the drug delivery compositions and devices do not include T cells. In certain embodiments, the additional pharmaceutical agent is not adoptively transferred cells. In certain embodiments, the additional pharmaceutical agent is not T cells. In certain embodiments, the drug delivery compositions and devices do not include tumor antigens. In certain embodiments, the drug delivery compositions and devices do not include tumor antigens loaded ex vivo.

In certain embodiments, “drug delivery composition” refers to the composition in a liquid form (e.g., a viscous solution). In certain embodiments, the term “drug delivery device” refers to the composition in a solid form (e.g., a hydrogel). In certain embodiments, the transition from composition to device may occur upon sufficient cross-linking such that the resulting material has a storage modulus consistent with a solid form that allows it to be physically manipulated and implanted in a surgical procedure. Accordingly, the drug delivery device, in its solid form, may be particularly amenable for carrying out an intended use of the present disclosure (e.g., surgical implantation).

In certain embodiments, the drug delivery composition and/or drug delivery device is prepared just prior to in vivo implantation (e.g., in an operating room or close proximity). In certain embodiments, the drug delivery composition and/or drug delivery device is prepared within 24 hours, 18 hours, 12 hours, 11 hours, 10 hours, 9 hours, 8 hours, 7 hours, 6 hours, 5 hours, 4 hours, 3 hours, 2 hours, 1 hour, 30 minutes, 20 minutes, 10 minutes, 5 minutes, or 1 minute of in vivo implantation.

In certain embodiments, the drug delivery composition and/or drug delivery device is prepared in advance of in vivo implantation. In certain embodiments, the drug delivery composition and/or drug delivery device is prepared within 31 days, 28 days, 21 days, 14 days, 7 days, 6 days, 5 days, 4 days, 3 days, 2 days, or 1 day of in vivo implantation.

In certain embodiments, the drug delivery composition is prepared within 1 year, 10 months, 8 months, 6 months, 4 months, 3 months, 2 months, 31 days, 28 days, 21 days, 14 days, 7 days, 6 days, 5 days, 4 days, 3 days, 2 days, or 1 day of its use in a therapeutic setting. In certain embodiments, the prepared drug delivery composition is then used to prepare the corresponding drug delivery device by addition of a cross-linking agent, as described herein, within 31 days, 28 days, 21 days, 14 days, 7 days, 6 days, 5 days, 4 days, 3 days, 2 days, 1 day, 18 hours, 12 hours, 11 hours, 10 hours, 9 hours, 8 hours, 7 hours, 6 hours, 5 hours, 4 hours, 3 hours, 2 hours, 1 hour, 30 minutes, 20 minutes, 10 minutes, 5 minutes, or 1 minute of in vivo implantation.

Also encompassed by the disclosure are kits. The kits provided may comprise compositions and/or devices described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the kit comprises precursor components (e.g., hyaluronic acid and a cross-linker; or alginate and a cross-linker) to the drug delivery composition and/or drug delivery device.

In certain embodiments, the kit comprises a hydrogel and an inhibitor of a proinflammatory pathway (e.g., an inhibitor of a proinflammatory immune response mediated by a p38 MAPK pathway). In certain embodiments, the kit comprises a hydrogel, an inhibitor of a proinflammatory pathway (e.g., an inhibitor of a proinflammatory immune response mediated by a p38 MAPK pathway), and an activator of innate immune response. In certain embodiments, the kit comprises a hydrogel, an inhibitor of a proinflammatory pathway (e.g., an inhibitor of a proinflammatory immune response mediated by a p38 MAPK pathway), and a cytokine. In certain embodiments, the kit comprises a hydrogel, an inhibitor of a proinflammatory pathway (e.g., an inhibitor of a proinflammatory immune response mediated by a p38 MAPK pathway), and an activator of adaptive immune response. In certain embodiments, the kit further comprises an activator of innate immune function. In certain embodiments, the kit further comprises a cytokine. In certain embodiments, the kit further comprises an activator of adaptive immune response. In certain embodiments, the kit further comprises a modulator of macrophage effector function. In certain embodiments, the kit further comprises an additional activator of adaptive immune response. In certain embodiments, the kit further comprises an oncolytic virus, a radioactive isotope, an immunomodulatory chemotherapeutic agent, a targeted agent, or a combination thereof. In certain embodiments, the kit comprises any drug delivery composition described herein. In certain embodiments, the kit comprises any drug delivery device described herein.

In certain embodiments, the kit does not comprise a chemotherapeutic agent. In certain embodiments, the kit does not comprise a cytotoxic agent.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating cancer. A kit described herein may include one or more additional pharmaceutical agents described herein as a separate composition.

### Methods of Treatment and Uses

The present disclosure provides methods of using drug delivery compositions and devices described herein, for the treatment and/or prevention of a proliferative disease, such as cancer (e.g. a sarcoma, a carcinoma, a lymphoma, a germ cell tumor, or a blastoma), in a subject. In some embodiments, compositions and/or devices described herein are for use in treatment of a resectable tumor. In some embodiments, compositions and/or devices described herein are for use in treatment of lymphoma present in a spleen or a tissue outside of a lymphatic system, e.g., a thyroid or stomach.

In some embodiments, the drug delivery compositions and devices described herein are useful in treating cancer. In some embodiments, the drug delivery compositions and devices described herein are useful to delay the onset of, slow the progression of, or ameliorate the symptoms of cancer. In some embodiments, the drug delivery compositions and devices described herein are useful to prevent cancer. In some embodiments, the drug delivery compositions and devices described herein are useful to prevent primary tumor regrowth. In some embodiments, the drug delivery compositions and devices described herein are useful to prevent tumor metastasis. In some embodiments, the drug delivery compositions and devices described herein are administered in combination with other compounds, drugs, or therapeutic agents to treat cancer.

In certain embodiments, the cancer is a solid tumor. In certain embodiments, the cancer is a sarcoma, a carcinoma, a lymphoma, a germ cell tumor, a blastoma, or a combination thereof. In certain embodiments, the tumor is a sarcoma, a carcinoma, a lymphoma, a germ cell tumor, a blastoma, or a combination thereof.

In some embodiments, the drug delivery compositions and devices described herein are useful for treating a cancer including, but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyo sarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); parathryroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget's disease of the vulva), or any combination thereof.

In certain embodiments, the cancer is breast cancer. In certain embodiments, the cancer is skin cancer. In certain embodiments, the cancer is melanoma. In certain embodiments, the cancer is lung cancer. In certain embodiments, the cancer is kidney cancer. In certain embodiments, the cancer is liver cancer. In certain embodiments, the cancer is pancreatic cancer. In certain embodiments, the cancer is colorectal cancer. In certain embodiments, the cancer is bladder cancer. In certain embodiments, the cancer is lymphoma. In certain embodiments, the cancer is prostate cancer. In certain embodiments, the cancer is thyroid cancer.

In some embodiments, the drug delivery compositions and devices described herein are useful in treating adenocarcinoma, adrenal gland cancer, anal cancer, angiosarcoma, appendix cancer, bile duct cancer, bladder cancer, bone cancer, brain cancer, breast cancer, bronchus cancer, carcinoid tumor, cardiac tumor, cervical cancer, choriocarcinoma, chordoma, colorectal cancer, connective tissue cancer, craniopharyngioma, ductal carcinoma in situ, endotheliosarcoma, endometrial cancer, ependymoma, epithelial carcinoma, esophageal cancer, Ewing's sarcoma, eye cancer, familiar hypereosinophilia, gall bladder cancer, gastric cancer, gastrointestinal carcinoid tumor, gastrointestinal stromal tumor (GIST), germ cell cancer, head and neck cancer, hemangioblastoma, histiocytosis, Hodgkin lymphoma, hypopharynx cancer, inflammatory myofibroblastic tumors, intraepithelial neoplasms, immunocytic amyloidosis, Kaposi sarcoma, kidney cancer, liver cancer, lung cancer, leiomyosarcoma (LMS), melanoma, midline tract carcinoma, multiple endocrine neoplasia syndrome, muscle cancer, mesothelioma, myeloproliferative disorder (MPD), nasopharynx cancer, neuroblastoma, neurofibroma, neuroendocrine cancer, non-Hodgkin lymphoma, osteosarcoma, ovarian cancer, pancreatic cancer, paraneoplastic syndromes, parathryroid cancer, papillary adenocarcinoma, penile cancer, pharyngeal cancer, pheochromocytoma, pinealoma, pituitary cancer, pleuropulmonary blastoma, primitive neuroectodermal tumor (PNT), plasma cell neoplasia, prostate cancer, rectal cancer, retinoblastoma, rhabdomyosarcoma, salivary gland cancer, sebaceous gland carcinoma, skin cancer, small bowel cancer, small intestine cancer, soft tissue sarcoma, stomach cancer, sweat gland carcinoma, synovioma, testicular cancer, thymic cancer, thyroid cancer, urethral cancer, uterine cancer, vaginal cancer, vascular cancer, vulvar cancer, or a combination thereof.

In some embodiments, the drug delivery compositions and devices described herein are useful in treating and/or preventing solid tumors and metastases.

For example, in some embodiments, a method comprises administering a drug delivery composition or device described herein (e.g., comprising a hydrogel biomaterial and an inhibitor of a p38 MAPK pathway) to a target site in a subject who has recently undergone a tumor resection. In some embodiments, the target site is a tumor resection site. In some embodiments, the target site is a sentinel lymph node. In some embodiments, the target site is a draining lymph node. In some embodiments, a target site is a site at which cancer cells have been treated or killed by prior cancer therapy, e.g., chemotherapy or radiation.

In some embodiments, a drug delivery composition (e.g., comprising a hydrogel biomaterial and an inhibitor of a p38 MAPK pathway) administered to a target site is a pre-formed gel and it can be administered to the target site by implantation. In some embodiments, a drug delivery composition (e.g., comprising precursor component(s) of a hydrogel and an inhibitor of a p38 MAPK pathway) administered to a target site is in an injectable format (e.g., a liquid). In some embodiments, administration as described herein involves administration of one or more biomaterial (e.g., hydrogel) precursor components that interact or react in situ to form a gel composition as described herein; in some such embodiments, such interaction or reaction involves crosslinking which may, in some embodiments, occur spontaneously and in some embodiments may be triggered by application of an agent (e.g., a catalyst and/or a reactant) and/or a condition (e.g., one or more of heat, pH, pressure, electromagnetic radiation which may be at a particular wavelength, etc). In some embodiments, a biomaterial (e.g., hydrogel) can be cross-linked by attaching thiols (e.g., EXTRACEL®, HYSTEM®), methacrylates, hexadecylamides (e.g., HYMOVIS®), and/or tyramines (e.g., CORGEL®). In some embodiments, a biomaterial (e.g., hydrogel) can be cross-linked directly with formaldehyde (e.g., HYLAN-A®), divinylsulfone (DVS) (e.g., HYLAN-B®), 1,4-butanediol diglycidyl ether (BDDE) (e.g., RESTYLANE®), glutaraldehyde, and/or genipin (see, e.g., Khunmanee et al. “Crosslinking method of hyaluronic-based hydrogel for biomedical applications” J Tissue Eng. 8: 1-16 (2017)). In some embodiments, a biomaterial (e.g., hydrogel) is crosslinked with divinylsulfone (DVS) (e.g., HYLAN-B®).

In certain embodiments, the methods described herein include implanting (e.g., via administration of a biomaterial gel or a set of precursors thereof as described herein) in a subject an effective amount of the drug delivery composition or device described herein. In certain embodiments, the methods described herein include surgically implanting in a subject an effective amount of the drug delivery composition or device described herein. In certain embodiments, the methods described herein further comprise implanting the drug delivery composition or device after surgical resection of a tumor. In certain embodiments, the methods described herein further comprise implanting the drug delivery composition or device at the site of tumor resection. In certain embodiments, the methods described herein further comprise implanting the drug delivery composition or device in the void volume of the resected tumor. In certain embodiments, the methods described herein further comprise implanting the drug delivery composition or device in the tumor resection site during tumor resection surgery.

In certain embodiments, the methods described herein comprise administering (e.g., implanting) the drug delivery composition or device after removal of, by weight, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99% of the resected tumor. In certain embodiments, the methods described herein comprise administering (e.g., implanting) the drug delivery composition or device after removal of, by volume, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99% of the resected tumor.

In certain embodiments, the methods described herein do not comprise administering (e.g., implanting) the drug delivery composition or device to a site adjacent to a tumor. In certain embodiments, the methods described herein do not comprise administering (e.g., implanting) the drug delivery composition or device adjacent to a tumor without resection of the tumor.

In certain embodiments, the drug delivery compositions and devices described herein are administered in combination with one or more additional therapeutic agents described herein. In certain embodiments, the additional therapeutic agent is an anti-cancer agent.

In certain embodiments, the subject being treated is a mammal. In certain embodiments, the subject is a human. In certain embodiments, the subject is a human patient who has received neoadjuvant (pre-operative) chemotherapy. In certain embodiments, the subject is a human patient who has received neoadjuvant radiation therapy. In certain embodiments, the subject is a human patient who has received neoadjuvant chemotherapy and radiation therapy. In certain embodiments, the subject is a human patient who has received neoadjuvant molecular targeted therapy. In certain embodiments, the subject is a human patient who has received neoadjuvant immunotherapy, including immune checkpoint blockade (e.g., anti-CTLA-4, anti-PD-1, and/or anti-PD-L1). In certain embodiments, the subject is a human patient who has not received neoadjuvant immunotherapy, including immune checkpoint blockade (e.g., anti-CTLA-4, anti-PD-1, and/or anti-PD-L1). In certain embodiments, the subject is a human patient whose tumor has not objectively responded to neoadjuvant therapy (as defined by Response Evaluation Criteria in Solid Tumors (RECIST) or immune-related Response Criteria (irRC)) (e.g., stable disease, progressive disease). In certain embodiments, the subject is a human patient whose target lesion has objectively responded to neoadjuvant therapy (e.g., partial response, complete response). Non-target lesions may exhibit an incomplete response, stable disease, or progressive disease. In certain embodiments, the subject is a human patient who would be eligible to receive immunotherapy as a standard of care in the adjuvant (post-operative) setting. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal, such as a rodent, pig, dog, or non-human primate. In certain embodiments, the subject is a non-human transgenic animal such as a transgenic mouse or transgenic pig.

### EXAMPLES

In order that the invention described herein may be more fully understood, the following examples are set forth. It should be understood that these examples are for illustrative purposes only and are not to be construed as limiting this invention in any manner.

### Example 1. Preparation and Uses of Exemplary Drug Delivery Compositions

To prepare hydrogels according to some embodiments described herein, a crosslinkable hyaluronic acid (e.g., a thiol-modified hyaluronic acid such as GLYCOSIL® hyaluronic acid) and a chemical crosslinker (e.g., a thiol-reactive crosslinker such as polyethylene diacrylate, e.g., EXTRALINK® polyethylene glycol diacrylate cross-linker) were combined to form a hydrogel. The hydrogel was formed upon allowing the combined reagents to stand for at least 1 hour. The storage modulus of the hydrogel was measured with a rheometer. These are summarized in Table 1.

TABLE 1

| | GLYCOSIL ® | EXTRALINK ® | total | | storage |
|---|---|---|---|---|---|
| Hydrogel | (w/v.) | (w/v) | volume | Hydrogel size | modulus |
| 1 | 2.0%; 120 μL | 12.5%; 30 μL | 150 μL | Diameter: 9 mm | ~1500 Pa |
| | | | | Height: 3.2 mm | |

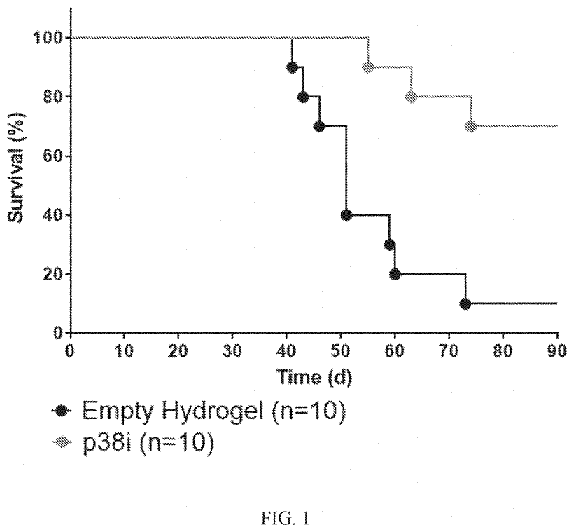

The hydrogels for FIG. 1 were prepared according to the methods described for hydrogel 1 in Table 1. 120 μL of a hyaluronic acid solution (2.0% w/v GLYCOSIL) was pipetted into a mold. One mg of a p38 MAPK inhibitor (e.g., losmapimod (Selleckchem)) was dissolved in 10 μL DMSO, and this solution was added to the hyaluronic acid solution in the mold. Following mixing to create a homogeneous distribution of the payload, 30 μL of a PEG-diacrylate crosslinker solution (12.5% w/v EXTRALINK) was pipetted into the mold. The crosslinked hydrogel solidified in the mold over the course of a few minutes. Female BALB/cJ mice were inoculated orthotopically with 4T1-Luc2 breast cancer cells in their fourth mammary fat pad. On day 10 post-tumor inoculation, tumors (˜100 mm³) were resected, and a hydrogel (e.g., a crosslinked hyaluronic acid hydrogel) loaded with a p38 MAPK inhibitor (e.g., losmapimod) was placed in the tumor resection site. Empty hydrogel was used as a negative control. Prolonged survival benefit was observed upon extended local release of a p38 inhibitor (FIG. 1).

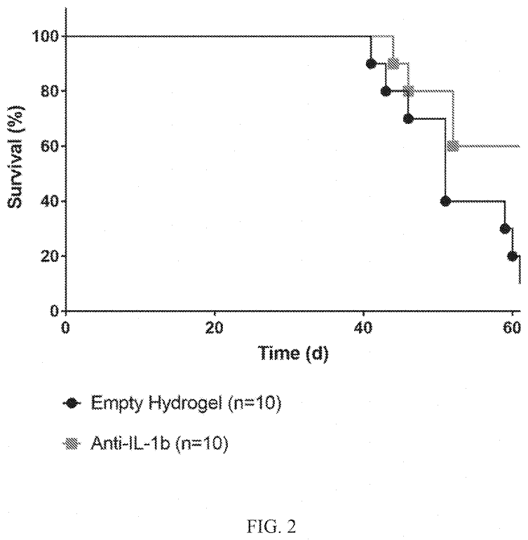

The hydrogels for FIG. 2 were prepared according to the methods described for hydrogel 1 in Table 1. 120 μL of a hyaluronic acid solution (2.0% w/v GLYCOSIL) was pipetted into a mold. Five hundred μg (40 μL) of an anti-IL-1β antibody (e.g., clone B122 (BioXCell)) was added to the hyaluronic acid solution in the mold. Following mixing to create a homogeneous distribution of the payload, 30 μL of a PEG-diacrylate crosslinker solution (12.5% w/v EXTRALINK) was pipetted into the mold. The crosslinked hydrogel solidified in the mold over the course of a few minutes. Female BALB/cJ mice were inoculated orthotopically with 4T1-Luc2 breast cancer cells in their fourth mammary fat pad. On day 10 post-tumor inoculation, tumors (˜100 mm³) were resected, and a hydrogel (e.g., a crosslinked hyaluronic acid hydrogel) loaded with an anti-IL-1β antibody (e.g., clone B122) was placed in the tumor resection site. Empty hydrogel was used as a negative control. Prolonged survival benefit was observed upon extended local release of an anti-IL-1β antibody (FIG. 2).

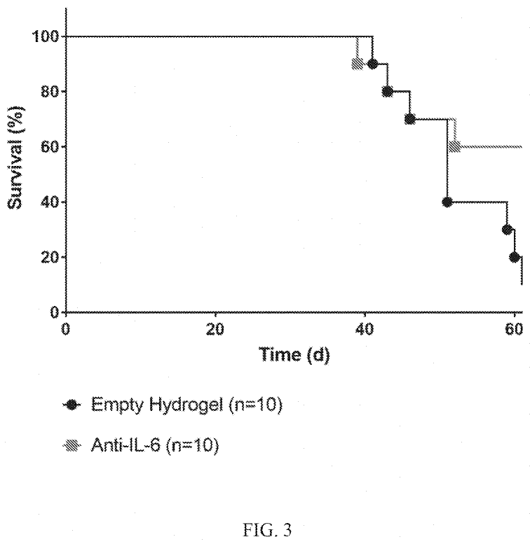

The hydrogels for FIG. 3 were prepared according to the methods described for hydrogel 1 in Table 1. 120 μL of a hyaluronic acid solution (2.0% w/v GLYCOSIL) was pipetted into a mold. Five hundred μL (40 μL) of an anti-IL-6 antibody (e.g., clone MP5-20F3 (BioXCell)) was added to the hyaluronic acid solution in the mold. Following mixing to create a homogeneous distribution of the payload, 30 μL of a PEG-diacrylate crosslinker solution (12.5% w/v EXTRALINK) was pipetted into the mold. The crosslinked hydrogel solidified in the mold over the course of a few minutes. Female BALB/cJ mice were inoculated orthotopically with 4T1-Luc2 breast cancer cells in their fourth mammary fat pad. On day 10 post-tumor inoculation, tumors (˜100 mm³) were resected, and a hydrogel (e.g., a crosslinked hyaluronic acid hydrogel) loaded with an anti-IL-6 antibody (e.g., clone MP5-20F3) was placed in the tumor resection site. Empty hydrogel was used as a negative control. Prolonged survival benefit was observed upon extended local release of an anti-IL-6 antibody (FIG. 3).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The entire content of International Patent Publication No. WO 2018/045058 (e.g., compositions, devices, methods of preparation, methods of use, and kits) is incorporated herein by reference for the purposes described herein.

What is claimed is:

1. A method comprising a step of:
intraoperative administration at a tumor resection site of a subject suffering from cancer:
a composition comprising a biomaterial and an inhibitor of a proinflammatory immune response mediated by a p38 mitogen-activated protein kinase (MAPK) pathway.

2. The method of claim 1, wherein the biomaterial is characterized by a storage modulus of about 500 Pa to about 50,000 Pa.

3. The method of claim 1, wherein the step of administration does not involve adoptive transfer of T cells to the subject.

4. The method of claim 1, wherein the step of administration does not involve administration of a tumor antigen to the subject.

5. The method of claim 1, wherein the step of administration does not involve administration of a microparticle to the subject.

6. The method of claim 1, wherein the biomaterial is or comprises a hydrogel.

7. The method of claim 6, wherein the biomaterial is or comprises hyaluronic acid.

8. The method of claim 7, wherein the biomaterial is or comprises a crosslinked hyaluronic acid.

9. The method of claim 8, wherein the biomaterial is or comprises a hyaluronic acid crosslinked with a polyethylene glycol crosslinker.

10. The method of claim 1, wherein the inhibitor is or comprises a p38 MAPK inhibitor that binds to an ATP and/or allosteric binding site of a p38 MAPK.

11. The method of claim 1, wherein the inhibitor is or comprises a p38 α/β MAPK inhibitor that binds to an ATP and/or allosteric binding site of a p38 MAPK.

12. The method of claim 11, wherein the p38 α/β MAPK inhibitor is or comprises losmapimod.

13. The method of claim 1, wherein the composition further comprises an activator of innate immunity.

14. The method of claim 13, wherein the activator of innate immunity is or comprises a stimulator of interferon genes (STING) agonist.

15. The method of claim 13, wherein the activator of innate immunity is or comprises a Toll-like receptor (TLR) 7 and/or TLR8 (“TLR7/8”) agonist.

16. The method of claim 1, wherein the composition further comprises an activator of adaptive immunity and/or a cytokine that modulates T cells, natural killer (NK) cells, monocytes, and/or dendritic cells.

17. The method of claim 1, wherein the composition further comprises a cytokine that modulates T cells, NK cells, monocytes, and/or dendritic cells; and the cytokine is selected from an IL-15 superagonist, IFN-α, IFN-β, IFN-γ, and combinations thereof.

18. The method of claim 1, wherein the composition further comprises a COX inhibitor.

19. The method of claim 1, wherein the composition further comprises a COX-2 inhibitor.

20. The method of claim 1, wherein the biomaterial forms a matrix or depot and the inhibitor is within the biomaterial.

21. The method of claim 20, wherein the inhibitor is released by diffusion through the biomaterial.

22. The method of claim 1, wherein the biomaterial is biodegradable in vivo.

23. The method of claim 1, wherein the biomaterial is characterized in that, when tested in vivo by implanting a biomaterial at a mammary fat pad of a mouse subject, less than or equal to 10% of the biomaterial remains in vivo 4 months after the implantation.

24. The method of claim 1, wherein the biomaterial is characterized in that, when tested in vitro by placing a composition comprising a biomaterial and losmapimod in PBS (pH 7.4), less than 100% of the losmapimod is released within 3 hours from the biomaterial.

25. The method of claim 1, wherein the biomaterial is characterized in that, when tested in vivo by implanting a composition comprising a biomaterial and losmapimod at a mammary fat pad of a mouse subject, less than or equal to 50% of the losmapimod is released in vivo 8 hours after the implantation.

26. The method of claim 1, wherein the biomaterial is characterized in that it extends release of the inhibitor so that, when assessed at 24 hours after administration, more inhibitor is present in the tumor resection site than is observed when the inhibitor is administered in solution.

27. The method of claim 1, wherein the administration is by implantation.

28. The method of claim 1, wherein the administration is by injection.

29. The method of claim 28, wherein the administration comprises injecting one or more precursor components of the biomaterial and permitting the biomaterial to form at the tumor resection site.

30. The method of claim 1, wherein the tumor resection site is characterized by absence of gross residual tumor antigen.

31. The method of claim 1, wherein the cancer is metastatic cancer.

32. The method of claim 31, further comprising a step of monitoring at least one metastatic site in the subject after the administration.
